SEQUENCE LISTING (89 sequences)

SEQ ID NO: 1 - protein, 295 aa, Artificial Sequence
  Description of Artificial Sequence Synthetic polypeptide
MEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDNSIRPEELLQMELLLVNKLKWNLAAMTPHDFIEHFLSKMPEAEENKQIIRKHAQAFVALCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACAPTDVRDVDI

SEQ ID NO: 2 - protein, 303 aa, Artificial Sequence
  Description of Artificial Sequence Synthetic polypeptide
MATSRYEPVAEIGVGAYGTVYKARDPHSGHFVALKSVRVPNGGGGGGGLPISTVREVALLRRLEAFEHPNVVRLMDVCATSRTDREIKVTLVFEHVDQDLRTYLDKAPPPGLPAETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFPPRGPRPVQSVVPEMEESGAQLLLEMLTFNPHKRISAFRALQHSYLHKDEGNPE

SEQ ID NO: 3 - protein, 18 aa, Artificial Sequence
  Description of Artificial Sequence Synthetic peptide
ASKGGGGSGGGGSGGGGS

SEQ ID NO: 4 - protein, 17 aa, Artificial Sequence
  Description of Artificial Sequence Synthetic peptide
ASKGGGGSLEVLFQPSR

SEQ ID NO: 5 - protein, 18 aa, Artificial Sequence
  Description of Artificial Sequence Synthetic peptide
ASKGGGGSLEVLFQGPSR

SEQ ID NO: 6 - protein, 15 aa, Artificial Sequence
  Description of Artificial Sequence Synthetic peptide
GGGGSGGGGSGGGGS

SEQ ID NO: 7 - protein, 616 aa, Artificial Sequence
  Description of Artificial Sequence Synthetic polypeptide
MEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDGSIRPEELLQMELLLVNKLKWNLAAMTPHDFIEHFLSKMPEAEENKQIIRKHAQTFVASCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACTPTDVRDVDIASKGGGGSGGGGSGGGGSMATSRYEPVAEIGVGAYGTVYKARDPHSGHFVALKSVRVPNGGGGGGGLPISTVREVALLRRLEAFEHPNVVRLMDVCATSRTDREIKVTLVFEHVDQDLRTYLDKAPPPGLPAETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFPPRGPRPVQSVVPEMEESGAQLLLEMLTFNPHKRISAFRALQHSYLHKDEGNPE

SEQ ID NO: 8 - protein, 615 aa, Artificial Sequence
  Description of Artificial Sequence Synthetic polypeptide
MEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDGSIRPEELLQMELLLVNKLKWNLAAMTPHDFIEHFLSKMPEAEENKQIIRKHAQTFVASCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACTPTDVRDVDIASKGGGGSLEVLFQPSRMATSRYEPVAEIGVGAYGTVYKARDPHSGHFVALKSVRVPNGGGGGGGLPISTVREVALLRRLEAFEHPNVVRLMDVCATSRTDREIKVTLVFEHVDQDLRTYLDKAPPPGLPAETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFPPRGPRPVQSVVPEMEESGAQLLLEMLTFNPHKRISAFRALQHSYLHKDEGNPE

SEQ ID NO: 9 - protein, 613 aa, Artificial Sequence
  Description of Artificial Sequence Synthetic polypeptide
MELLCCEGTRHAPRAGPDPRLLGDQRVLQSLLRLEERYVPRASYFQCVQREIKPHMRKMLAYWMLEVCEEQRCEEEVFPLAMNYLDRYLSCVPTRKAQLQLLGAVCMLLASKLRETTPLTIEKLCIYTDHAVSPRQLRDWEVLVLGKLKWDLAAVIAHDFLAFILHRLSLPRDRQALVKKHAQTFLALCATDYTFAMYPPSMIATGSIGAAVQGLGACSMSGDELTELLAGITGTEVDCLRACQEQIEAALRESLREASQTSSSPAPKAPRGSSSQGPSQTSTPTDVTAIHLASKGGGGSGGGGSGGGGSMATSRYEPVAEIGVGAYGTVYKARDPHSGHFVALKSVRVPNGGGGGGGLPISTVREVALLRRLEAFEHPNVVRLMDVCATSRTDREIKVTLVFEHVDQDLRTYLDKAPPPGLPAETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFPPRGPRPVQSVVPEMEESGAQLLLEMLTFNPHKRISAFRALQHSYLHKDEGNPE

SEQ ID NO: 10 - protein, 612 aa, Artificial Sequence
  Description of Artificial Sequence Synthetic polypeptide
MELLCCEGTRHAPRAGPDPRLLGDQRVLQSLLRLEERYVPRASYFQCVQREIKPHMRKMLAYWMLEVCEEQRCEEEVFPLAMNYLDRYLSCVPTRKAQLQLLGAVCMLLASKLRETTPLTIEKLCIYTDHAVSPRQLRDWEVLVLGKLKWDLAAVIAHDFLAFILHRLSLPRDRQALVKKHAQTFLALCATDYTFAMYPPSMIATGSIGAAVQGLGACSMSGDELTELLAGITGTEVDCLRACQEQIEAALRESLREASQTSSSPAPKAPRGSSSQGPSQTSTPTDVTAIHLASKGGGGSLEVLFQPSRMATSRYEPVAEIGVGAYGTVYKARDPHSGHFVALKSVRVPNGGGGGGGLPISTVREVALLRRLEAFEHPNVVRLMDVCATSRTDREIKVTLVFEHVDQDLRTYLDKAPPPGLPAETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFPPRGPRPVQSVVPEMEESGAQLLLEMLTFNPHKRISAFRALQHSYLHKDEGNPE

SEQ ID NO: 11 - protein, 319 aa, Artificial Sequence
  Description of Artificial Sequence Synthetic polypeptide
APRPSNKRLQQSGMPRPSNKRLQQENLYFQGQLENLYFQGPPAPPQMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKDYDIPTTASENLAAQGEGGGLNDIFEAQKIEWHE

SEQ ID NO: 12 - protein, 7 aa, Artificial Sequence
  Description of Artificial Sequence Synthetic peptide
ENLYFQG

SEQ ID NO: 13 - protein, 10 aa, Artificial Sequence
  Description of Artificial Sequence Synthetic peptide
PRPSNKRLQQ

SEQ ID NO: 14 - protein, 15 aa, Artificial Sequence
  Description of Artificial Sequence Synthetic peptide
GLNDIFEAQKIEWHE

SEQ ID NO: 15 - DNA, 1708 bp, Artificial Sequence
  Description of Artificial Sequence Synthetic polynucleotide
ggatccaatgaaggataacaccgtgccactgaaattgattgccctgttagcgaacggtgaatttcactctggcgagcagttgggtgaaacgctgggaatgagccgggcggctattaataaacacattcagacactgcgtgactggggcgttgatgtctttaccgttccgggtaaaggatacagcctgcctgagcctatccagttacttaatgctaaacagatattgggtcagctggatggcggtagtgtagccgtgctgccagtgattgactccacgaatcagtaccttcttgatcgtatcggagagcttaaatcgggcgatgcttgcattgcagaataccagcaggctggccgtggtcgccggggtcggaaatggttttcgccttttggcgcaaacttatatttgtcgatgttctggcgtctggaacaaggcccggcggcggcgattggtttaagtctggttatcggtatcgtgatggcggaagtattacgcaagctgggtgcagataaagttcgtgttaaatggcctaatgacctctatctgcaggatcgcaagctggcaggcattctggtggagctgactggcaaaactggcgatgcggcgcaaatagtcattggagccgggatcaacatggcaatgcgccgtgttgaagagagtgtcgttaatcaggggtggatcacgctgcaggaagcggggatcaatctcgatcgtaatacgttggcggccatgctaatacgtgaattacgtgctgcgttggaactcttcgaacaagaaggattggcaccttatctgtcgcgctgggaaaagctggataattttattaatcgcccagtgaaacttatcattggtgataaagaaatatttggcatttcacgcggaatagacaaacagggggctttattacttgagcaggatggaataataaaaccctggatgggcggtgaaatatccctgcgtagtgcagaaaaaaggagcatcgccacaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 16 - protein, 566 aa, Artificial Sequence
  Description of Artificial Sequence Synthetic polypeptide
MKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEKRSIATPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 17 - DNA, 1626 bp, Artificial Sequence
  Description of Artificial Sequence Synthetic polynucleotide
ggatccaatgaaggataacaccgtgccactgaaattgattgccctgttagcgaacggtgaatttcactctggcgagcagttgggtgaaacgctgggaatgagccgggcggctattaataaacacattcagacactgcgtgactggggcgttgatgtctttaccgttccgggtaaaggatacagcctgcctgagcctatccagttacttaatgctaaacagatattgggtcagctggatggcggtagtgtagccgtgctgccagtgattgactccacgaatcagtaccttcttgatcgtatcggagagcttaaatcgggcgatgcttgcattgcagaataccagcaggctggccgtggtcgccggggtcggaaatggttttcgccttttggcgcaaacttatatttgtcgatgttctggcgtctggaacaaggcccggcggcggcgattggtttaagtctggttatcggtatcgtgatggcggaagtattacgcaagctgggtgcagataaagttcgtgttaaatggcctaatgacctctatctgcaggatcgcaagctggcaggcattctggtggagctgactggcaaaactggcgatgcggcgcaaatagtcattggagccgggatcaacatggcaatgcgccgtgttgaagagagtgtcgttaatcaggggtggatcacgctgcaggaagcggggatcaatctcgatcgtaatacgttggcggccatgctaatacgtgaattacgtgctgcgttggaactcttcgaacaagaaggattggcaccttatctgtcgcgctgggaaaagctggataattttattaatcgcccagtgaaacttatcattggtgataaagaaatatttggcatttcacgcggaatagacaaacagggggctttattacttgagcaggatggaataataaaaccctggatgggcggtgaaatatccctgcgtagtgcagaaaaaaggagcatcgccacaccggtcgccaccatggccaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggctcgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgctggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagctaagcacaattcgagctcggtacc

SEQ ID NO: 18 - protein, 530 aa, Artificial Sequence
  Description of Artificial Sequence Synthetic polypeptide
MKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEKRSIATPVATMATEYKPTVRLATRDDVPRAVRTLAAAFADYPATRHTVDPDRHIERVTELQELFLTRVGLDIGKVWVADDGAAVAVWTTPESVEAGAVFAEIGSRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 19 - DNA, 5570 bp, Artificial Sequence
  Description of Artificial Sequence Synthetic polynucleotide
gggacccgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgggacccgacaggcccgaaggaatagaagaagaaggtggagagagagacagagacagatccattcgattagtgaacggatctcgacggtatcgatcacgagactagcctcgaccatcgatgtcgatcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaaatttaattaatctcgacggtatcggttaacttttaaaagaaaaggggggattggggggtacagtgcaggggaaagaatagtagacataatagcaacagacatacaaatttaaagaattacaaaaacaaattacaaaaattcaaaattttatcgatcacgagactagcctcgaggtttaaactacgggatctcgaggatctcgaattcaaggatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctccgttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgatctagagggcccgcggttcgaaggtaagcctatccctaaccctctcctcggtctcgattctacgcgtaccggtcatcatcaccatcaccattgagtttaaactacgggctgcaggaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctagctagctagtcgagctcaacttcgaattcgatatcaagcttatcgcgataccgtcgac

SEQ ID NO: 20 - DNA, 6967 bp, Artificial Sequence
  Description of Artificial Sequence Synthetic polynucleotide
gggacccgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgggacccgacaggcccgaaggaatagaagaagaaggtggagagagagacagagacagatccattcgattagtgaacggatctcgacggtatcgatcacgagactagcctcgaccatcgatgtcgacgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaatttcgacatttaaatttaattaatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgaggatctcgaattcaaggatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtcgtcccaccggttactaccatggctccacgaccatccaacaaacgtctccagcagtccggtatgccacgaccctccaacaagcgtcttcaacaggagaacctttactttcaaggtcaattggaaaatctctatttccagggaccaccagcgccaccacagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggattatgatattccaactactgcaagcgagaatttggcagcacagggtgagggtggtggcctgaacgacatcttcgaggcccagaagatcgagtggcacgagtaagcggccgcaggagctcaacttcgaattcgataaactacgggctgcaggaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggaatctgatctggggcctcggtgcacatgctttacatgtgtttagtcgaggttaaaaaagctctaggccccccgaaccacggggacgtggttttcctttgaaaaacacgatgataagcttgccacaaccccgggatccaatgaaggataacaccgtgccactgaaattgattgccctgttagcgaacggtgaatttcactctggcgagcagttgggtgaaacgctgggaatgagccgggcggctattaataaacacattcagacactgcgtgactggggcgttgatgtctttaccgttccgggtaaaggatacagcctgcctgagcctatccagttacttaatgctaaacagatattgggtcagctggatggcggtagtgtagccgtgctgccagtgattgactccacgaatcagtaccttcttgatcgtatcggagagcttaaatcgggcgatgcttgcattgcagaataccagcaggctggccgtggtcgccggggtcggaaatggttttcgccttttggcgcaaacttatatttgtcgatgttctggcgtctggaacaaggcccggcggcggcgattggtttaagtctggttatcggtatcgtgatggcggaagtattacgcaagctgggtgcagataaagttcgtgttaaatggcctaatgacctctatctgcaggatcgcaagctggcaggcattctggtggagctgactggcaaaactggcgatgcggcgcaaatagtcattggagccgggatcaacatggcaatgcgccgtgttgaagagagtgtcgttaatcaggggtggatcacgctgcaggaagcggggatcaatctcgatcgtaatacgttggcggccatgctaatacgtgaattacgtgctgcgttggaactcttcgaacaagaaggattggcaccttatctgtcgcgctgggaaaagctggataattttattaatcgcccagtgaaacttatcattggtgataaagaaatatttggcatttcacgcggaatagacaaacagggggctttattacttgagcaggatggaataataaaaccctggatgggcggtgaaatatccctgcgtagtgcagaaaaaaggagcatcgccacaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagcggccgcgactctagaatttactagtcatatg

SEQ ID NO: 21 - DNA, 6851 bp, Artificial Sequence
  Description of Artificial Sequence Synthetic polynucleotide
gggacccgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgggacccgacaggcccgaaggaatagaagaagaaggtggagagagagacagagacagatccattcgattagtgaacggatctcgacggtatcgatcacgagactagcctcgaccatcgatgtcgacgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaatttcgacatttaaatttaattaatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgaggatctcgaattcaaggatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtcgtcccaccggttactaccatggctccacgaccatccaacaaacgtctccagcagtccggtatgccacgaccctccaacaagcgtcttcaacaggagaacctttactttcaaggtcaattggaaaatctctatttccagggaccaccagcgccaccacagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggattatgatattccaactactgcaagcgagaatttggcagcacagggtgagggtggtggcctgaacgacatcttcgaggcccagaagatcgagtggcacgagtaagcggccgcaggagctcaacttcgaattcgataaactacgggctgcaggaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggaatctgatctggggcctcggtgcacatgctttacatgtgtttagtcgaggttaaaaaagctctaggccccccgaaccacggggacgtggttttcctttgaaaaacacgatgataagcttgccacaaccccgggatccaatgaaggataacaccgtgccactgaaattgattgccctgttagcgaacggtgaatttcactctggcgagcagttgggtgaaacgctgggaatgagccgggcggctattaataaacacattcagacactgcgtgactggggcgttgatgtctttaccgttccgggtaaaggatacagcctgcctgagcctatccagttacttaatgctaaacagatattgggtcagctggatggcggtagtgtagccgtgctgccagtgattgactccacgaatcagtaccttcttgatcgtatcggagagcttaaatcgggcgatgcttgcattgcagaataccagcaggctggccgtggtcgccggggtcggaaatggttttcgccttttggcgcaaacttatatttgtcgatgttctggcgtctggaacaaggcccggcggcggcgattggtttaagtctggttatcggtatcgtgatggcggaagtattacgcaagctgggtgcagataaagttcgtgttaaatggcctaatgacctctatctgcaggatcgcaagctggcaggcattctggtggagctgactggcaaaactggcgatgcggcgcaaatagtcattggagccgggatcaacatggcaatgcgccgtgttgaagagagtgtcgttaatcaggggtggatcacgctgcaggaagcggggatcaatctcgatcgtaatacgttggcggccatgctaatacgtgaattacgtgctgcgttggaactcttcgaacaagaaggattggcaccttatctgtcgcgctgggaaaagctggataattttattaatcgcccagtgaaacttatcattggtgataaagaaatatttggcatttcacgcggaatagacaaacagggggctttattacttgagcaggatggaataataaaaccctggatgggcggtgaaatatccctgcgtagtgcagaaaaaaggagcatcgccacaccggtcgccaccatggccaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggctcgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgctggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagctaagcacaattcgagctcggtacc

SEQ ID NO: 22 - DNA, 13749 bp, Artificial Sequence
  Description of Artificial Sequence Synthetic polynucleotide
cagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgggacccgacaggcccgaaggaatagaagaagaaggtggagagagagacagagacagatccattcgattagtgaacggatctcgacggtatcgatcacgagactagcctcgaccatcgatgtcgatcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaaatttaattaatctcgacggtatcggttaacttttaaaagaaaaggggggattggggggtacagtgcaggggaaagaatagtagacataatagcaacagacatacaaatttaaagaattacaaaaacaaattacaaaaattcaaaattttatcgatcacgagactagcctcgaggtttaaactacgggatctcgaggatctcgaattcaaggatcacaagtttgtacaaaaaagcaggctccgcggccgcccccttcaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtctagaaagggtgggcgcgccgacccagctttcttgtacaaagtggttgatctagagggcccgcggttcgaaggtaagcctatccctaaccctctcctcggtctcgattctacgcgtaccggtcatcatcaccatcaccattgagtttaaactacgggctgcaggaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctagctagctagtcgagctcaacttcgaattcgatatcaagcttatcgcgataccgtcgacctcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgctcaagcttcgaattaattctgcagagctcggtacctttaagaccaatgacttacaaggcagctgtagatcttagccactttttaaaagaaaaggggggactggaagggctaattcactcccaacgaagacaagatgggatcaattcaccatgggaataacttcgtatagcatacattatacgaagttatgctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagt

SEQ ID NO: 23 - DNA, 3465 bp, Artificial Sequence
  Description of Artificial Sequence Synthetic polynucleotide
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcccccttcaccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcaggccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccagactgcctccgggcctgccaggagcagatcgaagctctgctggagtcaagcctgcgccaggcccagcagaacatggaccccaaggccgccgaggaggaggaagaggaggaggaggaggtggacctggcttgcgcacccaccgacgtgcgggacgtggacatcaagggtgggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 24 - DNA, 4428 bp, Artificial Sequence
  Description of Artificial Sequence Synthetic polynucleotide
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcccccttcaccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacggctccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgcctcttgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccagactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatcgccagcaagggcggcggcggcagcggcggcggcggcagcggcggcggcggcagcatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagaagggtgggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 25 - DNA, 4425 bp, Artificial Sequence
  Description of Artificial Sequence Synthetic polynucleotide
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcccccttcaccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacggctccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgcctcttgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccagactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatcgccagcaagggcggcggcggcagcctggaggtgctgttccagcccagccgcatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagaagggtgggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 26 - DNA, 4419 bp, Artificial Sequence
  Description of Artificial Sequence Synthetic polynucleotide
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcccccttcaccatggagctgctgtgttgcgaaggcacccggcacgcgccccgggccgggccggacccgcggctgctgggggaccagcgtgtcctgcagagcctgctccgcctggaggagcgctacgtaccccgcgcctcctacttccagtgcgtgcagcgggagatcaagccgcacatgcggaagatgctggcttactggatgctggaggtatgtgaggagcagcgctgtgaggaggaagtcttccccctggccatgaactacctggatcgctacctgtcttgcgtccccacccgaaaggcgcagttgcagctcctgggtgcggtctgcatgctgctggcctccaagctgcgcgagaccacgcccctgaccatcgaaaaactgtgcatctacaccgaccacgctgtctctccccgccagttgcgggactgggaggtgctggtcctagggaagctcaagtgggacctggctgctgtgattgcacatgatttcctggccttcattctgcaccggctctctctgccccgtgaccgacaggccttggtcaaaaagcatgcccagacctttttggccctctgtgctacagattatacctttgccatgtacccgccatccatgatcgccacgggcagcattggggctgcagtgcaaggcctgggtgcctgctccatgtccggggatgagctcacagagctgctggcagggatcactggcactgaagtggactgcctgcgggcctgtcaggagcagatcgaagctgcactcagggagagcctcagggaagcctctcagaccagctccagcccagcgcccaaagccccccggggctccagcagccaagggcccagccagaccagcactcctacagatgtcacagccatacacctggccagcaagggcggcggcggcagcggcggcggcggcagcggcggcggcggcagcatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagaagggtgggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 27 - DNA, 4416 bp, Artificial Sequence
  Description of Artificial Sequence Synthetic polynucleotide
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcccccttcaccatggagctgctgtgttgcgaaggcacccggcacgcgccccgggccgggccggacccgcggctgctgggggaccagcgtgtcctgcagagcctgctccgcctggaggagcgctacgtaccccgcgcctcctacttccagtgcgtgcagcgggagatcaagccgcacatgcggaagatgctggcttactggatgctggaggtatgtgaggagcagcgctgtgaggaggaagtcttccccctggccatgaactacctggatcgctacctgtcttgcgtccccacccgaaaggcgcagttgcagctcctgggtgcggtctgcatgctgctggcctccaagctgcgcgagaccacgcccctgaccatcgaaaaactgtgcatctacaccgaccacgctgtctctccccgccagttgcgggactgggaggtgctggtcctagggaagctcaagtgggacctggctgctgtgattgcacatgatttcctggccttcattctgcaccggctctctctgccccgtgaccgacaggccttggtcaaaaagcatgcccagacctttttggccctctgtgctacagattatacctttgccatgtacccgccatccatgatcgccacgggcagcattggggctgcagtgcaaggcctgggtgcctgctccatgtccggggatgagctcacagagctgctggcagggatcactggcactgaagtggactgcctgcgggcctgtcaggagcagatcgaagctgcactcagggagagcctcagggaagcctctcagaccagctccagcccagcgcccaaagccccccggggctccagcagccaagggcccagccagaccagcactcctacagatgtcacagccatacacctggccagcaagggcggcggcggcagcctggaggtgctgttccagcccagccgcatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagaagggtgggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 28 - DNA, 25 bp, Artificial Sequence
  Description of Artificial Sequence Synthetic oligonucleotide
caccatggaacaccagctcctgtgc

SEQ ID NO: 29 - DNA, 21 bp, Artificial Sequence
  Description of Artificial Sequence Synthetic oligonucleotide
tcagatgtccacgtcccgcac

SEQ ID NO: 30 - DNA, 25 bp, Artificial Sequence
  Description of Artificial Sequence Synthetic oligonucleotide
caccatggagctgctgtgttgcgaa

SEQ ID NO: 31 - DNA, 76 bp, Artificial Sequence
  Description of Artificial Sequence Synthetic oligonucleotide
gctgccgccgccgccgctgccgccgccgccgctgccgccgccgcccttgctggccaggtgtatggctgtgacatct

SEQ ID NO: 32 - DNA, 18 bp, Artificial Sequence
  Description of Artificial Sequence Synthetic oligonucleotide
gctgccgccgccgccgct

SEQ ID NO: 33 - DNA, 60 bp, Artificial Sequence
  Description of Artificial Sequence Synthetic oligonucleotide
ctgccgccgccgccgctgccgccgccgcccttgctggccaggtgtatggctgtgacatct

SEQ ID NO: 34 - DNA, 18 bp, Artificial Sequence
  Description of Artificial Sequence Synthetic oligonucleotide
ctgccgccgccgccgctg

SEQ ID NO: 35 - DNA, 73 bp, Artificial Sequence
  Description of Artificial Sequence Synthetic oligonucleotide
gcggctgggctggaacagcacctccaggctgccgccgccgcccttgctggccaggtgtatggctgtgacatct

SEQ ID NO: 36 - DNA, 18 bp, Artificial Sequence
  Description of Artificial Sequence Synthetic oligonucleotide
gcggctgggctggaacag

SEQ ID NO: 37 - DNA, 58 bp, Artificial Sequence
  Description of Artificial Sequence Synthetic oligonucleotide
cagcacctccaggctgccgccgccgcccttgctggccaggtgtatggctgtgacatct

SEQ ID NO: 38 - DNA, 18 bp, Artificial Sequence
  Description of Artificial Sequence Synthetic oligonucleotide
cagcacctccaggctgcc

SEQ ID NO: 39 - DNA, 75 bp, Artificial Sequence
  Description of Artificial Sequence Synthetic oligonucleotide
gccagcaagggcggcggcggcagcggcggcggcggcagcggcggcggcggcagcatggctacctctcgatatgag

SEQ ID NO: 40 - DNA, 21 bp, Artificial Sequence
  Description of Artificial Sequence Synthetic oligonucleotide
gccagcaagggcggcggcggc

SEQ ID NO: 41 - DNA, 57 bp, Artificial Sequence
  Description of Artificial Sequence Synthetic oligonucleotide
ggcagcggcggcggcggcagcggcggcggcggcagcatggctacctctcgatatgag

SEQ ID NO: 42 - DNA, 21 bp, Artificial Sequence
  Description of Artificial Sequence Synthetic oligonucleotide
ggcagcggcggcggcggcagc

SEQ ID NO: 43 - DNA, 72 bp, Artificial Sequence
  Description of Artificial Sequence Synthetic oligonucleotide
gccagcaagggcggcggcggcagcctggaggtgctgttccagcccagccgcatggctacctctcgatatgag

SEQ ID NO: 44 - DNA, 18 bp, Artificial Sequence
  Description of Artificial Sequence Synthetic oligonucleotide
gccagcaagggcggcggc

SEQ ID NO: 45 - DNA, 57 bp, Artificial Sequence
  Description of Artificial Sequence Synthetic oligonucleotide
ggcggcagcctggaggtgctgttccagcccagccgcatggctacctctcgatatgag

SEQ ID NO: 46 - DNA, 18 bp, Artificial Sequence
  Description of Artificial Sequence Synthetic oligonucleotide
ggcggcagcctggaggtg

SEQ ID NO: 47 - DNA, 24 bp, Artificial Sequence
  Description of Artificial Sequence Synthetic oligonucleotide
caccatggaacaccagctcctgtg

SEQ ID NO: 48 - DNA, 75 bp, Artificial Sequence
  Description of Artificial Sequence Synthetic oligonucleotide
gctgccgccgccgccgctgccgccgccgccgctgccgccgccgcccttgctggcgatgtccacgtcccgcacgtc

SEQ ID NO: 49 - DNA, 59 bp, Artificial Sequence
  Description of Artificial Sequence Synthetic oligonucleotide
ctgccgccgccgccgctgccgccgccgcccttgctggcgatgtccacgtcccgcacgtc

SEQ ID NO: 50 - DNA, 72 bp, Artificial Sequence
  Description of Artificial Sequence Synthetic oligonucleotide
gcggctgggctggaacagcacctccaggctgccgccgccgcccttgctggcgatgtccacgtcccgcacgtc

SEQ ID NO: 51 - DNA, 57 bp, Artificial Sequence
  Description of Artificial Sequence Synthetic oligonucleotide
cagcacctccaggctgccgccgccgcccttgctggcgatgtccacgtcccgcacgtc

SEQ ID NO: 52 - DNA, 22 bp, Artificial Sequence
  Description of Artificial Sequence Synthetic oligonucleotide
caggtgtatggctgtgacatct

SEQ ID NO: 53 - DNA, 25 bp, Artificial Sequence
  Description of Artificial Sequence Synthetic oligonucleotide
caccatggaacaccagctcctgtgc

SEQ ID NO: 54 - DNA, 21 bp, Artificial Sequence
  Description of Artificial Sequence Synthetic oligonucleotide
gatgtccacgtcccgcacgtc

SEQ ID NO: 55 - DNA, 21 bp, Artificial Sequence
  Description of Artificial Sequence Synthetic oligonucleotide
tcagatgtccacgtcccgcac

SEQ ID NO: 56 - DNA, 21 bp, Artificial Sequence
  Description of Artificial Sequence Synthetic oligonucleotide
ctccggattaccttcatcctt

SEQ ID NO: 57 - DNA, 21 bp, Artificial Sequence
  Description of Artificial Sequence Synthetic oligonucleotide
ggctgtattcagctccgaggt

SEQ ID NO: 58 - DNA, 18 bp, Artificial Sequence
  Description of Artificial Sequence Synthetic oligonucleotide
gccagcaagggcggcggc

SEQ ID NO: 59 - DNA, 18 bp, Artificial Sequence
  Description of Artificial Sequence Synthetic oligonucleotide
gcggctggggccctggaa

SEQ ID NO: 60 - DNA, 75 bp, Artificial Sequence
  Description of Artificial Sequence Synthetic oligonucleotide
gccagcaagggcggcggcggcagcggcggcggcggcagcggcggcggcggcagcatggagaaggacggcctgtgc

SEQ ID NO: 61 - DNA, 57 bp, Artificial Sequence
  Description of Artificial Sequence Synthetic oligonucleotide
ggcagcggcggcggcggcagcggcggcggcggcagcatggagaaggacggcctgtgc

SEQ ID NO: 62 - DNA, 75 bp, Artificial Sequence
  Description of Artificial Sequence Synthetic oligonucleotide
gccagcaagggcggcggcggcagcctggaggtgctgttccagggccccagccgcatggagaaggacggcctgtgc

SEQ ID NO: 63 - DNA, 75 bp, Artificial Sequence
  Description of Artificial Sequence Synthetic oligonucleotide
gccagcaagggcggcggcggcagcctggaggtgctgttccagggccccagccgcatggctacctctcgatatgag

SEQ ID NO: 64 - DNA, 75 bp, Artificial Sequence
  Description of Artificial Sequence Synthetic oligonucleotide
gcggctggggccctggaacagcacctccaggctgccgccgccgcccttgctggcgatgtccacgtcccgcacgtc

SEQ ID NO: 65 - DNA, 76 bp, Artificial Sequence
  Description of Artificial Sequence Synthetic oligonucleotide
gcggctggggccctggaacagcacctccaggctgccgccgccgcccttgctggccaggtgtatggctgtgacatct

SEQ ID NO: 66 - DNA, 12132 bp, Artificial Sequence
  Description of Artificial Sequence Synthetic polynucleotide
ttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatgtcgacgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaatttcgacatttaaatttaattaatctcgacggtatcggttaacttttaaaagaaaaggggggattggggggtacagtgcaggggaaagaatagtagacataatagcaacagacatacaaactaaagaattacaaaaacaaattacaaaaattcaaaattttccgatcacgagactagcctcgaggtttgggatccaatgaaggataacaccgtgccactgaaattgattgccctgttagcgaacggtgaatttcactctggcgagcagttgggtgaaacgctgggaatgagccgggcggctattaataaacacattcagacactgcgtgactggggcgttgatgtctttaccgttccgggtaaaggatacagcctgcctgagcctatccagttacttaatgctaaacagatattgggtcagctggatggcggtagtgtagccgtgctgccagtgattgactccacgaatcagtaccttcttgatcgtatcggagagcttaaatcgggcgatgcttgcattgcagaataccagcaggctggccgtggtcgccggggtcggaaatggttttcgccttttggcgcaaacttatatttgtcgatgttctggcgtctggaacaaggcccggcggcggcgattggtttaagtctggttatcggtatcgtgatggcggaagtattacgcaagctgggtgcagataaagttcgtgttaaatggcctaatgacctctatctgcaggatcgcaagctggcaggcattctggtggagctgactggcaaaactggcgatgcggcgcaaatagtcattggagccgggatcaacatggcaatgcgccgtgttgaagagagtgtcgttaatcaggggtggatcacgctgcaggaagcggggatcaatctcgatcgtaatacgttggcggccatgctaatacgtgaattacgtgctgcgttggaactcttcgaacaagaaggattggcaccttatctgtcgcgctgggaaaagctggataattttattaatcgcccagtgaaacttatcattggtgataaagaaatatttggcatttcacgcggaatagacaaacagggggctttattacttgagcaggatggaataataaaaccctggatgggcggtgaaatatccctgcgtagtgcagaaaaaaggagcatcgccacaccggtcgccaccatggtgagcaagggcgaggaggataacatgggggtaaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggaatctgatctggggcctcggtgcacatgctttacatgtgtttagtcgaggttaaaaaagctctaggccccccgaaccacggggacgtggttttcctttgaaaaacacgatgataagcttgccacaaccccgggataattcctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagtcatatgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggtacgtatggccaggtacctttaagaccaatgacttacaaggcagctgtagatcttagccactttttaaaagaaaaggggggactggaagggctaattcactcccaacgaagacaagatgggatcaattcaccatgggaataacttcgtatagcatacattatacgaagttatgctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtag

SEQ ID NO: 67 - DNA, 12153 bp, Artificial Sequence
  Description of Artificial Sequence Synthetic polynucleotide
ttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatgtcgacgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaatttcgacatttaaatttaattaatctcgacggtatcggttaacttttaaaagaaaaggggggattggggggtacagtgcaggggaaagaatagtagacataatagcaacagacatacaaactaaagaattacaaaaacaaattacaaaaattcaaaattttccgatcacgagactagcctcgaggtttaaactacgggatctacgcgtaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgaccatgtctaggctggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgattttgaccttgacatgctccccgggtaaataacttcgtatagtatacattatacgaagttatggatccgcggccgcaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggccgcgactctagaatttactagtcatatgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggtacgtatggccaggtacctttaagaccaatgacttacaaggcagctgtagatcttagccactttttaaaagaaaaggggggactggaagggctaattcactcccaacgaagacaagatgggatcaattcaccatgggaataacttcgtatagcatacattatacgaagttatgctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtag

SEQ ID NO: 68 - DNA, 12823 bp, Artificial Sequence
  Description of Artificial Sequence Synthetic polynucleotide
cagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatggtccgaggttctagacgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcaccggtcactagtccagtgtggtggaattctgcagatatcaacaagtttgtacaaaaaagcaggctccgcggccgcccccttcaccatggagctgctgtgttgcgaaggcacccggcacgcgccccgggccgggccggacccgcggctgctgggggaccagcgtgtcctgcagagcctgctccgcctggaggagcgctacgtaccccgcgcctcctacttccagtgcgtgcagcgggagatcaagccgcacatgcggaagatgctggcttactggatgctggaggtatgtgaggagcagcgctgtgaggaggaagtcttccccctggccatgaactacctggatcgctacctgtcttgcgtccccacccgaaaggcgcagttgcagctcctgggtgcggtctgcatgctgctggcctccaagctgcgcgagaccacgcccctgaccatcgaaaaactgtgcatctacaccgaccacgctgtctctccccgccagttgcgggactgggaggtgctggtcctagggaagctcaagtgggacctggctgctgtgattgcacatgatttcctggccttcattctgcaccggctctctctgccccgtgaccgacaggccttggtcaaaaagcatgcccagacctttttggccctctgtgctacagattatacctttgccatgtacccgccatccatgatcgccacgggcagcattggggctgcagtgcaaggcctgggtgcctgctccatgtccggggatgagctcacagagctgctggcagggatcactggcactgaagtggactgcctgcgggcctgtcaggagcagatcgaagctgcactcagggagagcctcagggaagcctctcagaccagctccagcccagcgcccaaagccccccggggctccagcagccaagggcccagccagaccagcactcctacagatgtcacagccatacacctggccagcaagggcggcggcggcagcctggaggtgctgttccagggccccagccgcatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagaagggtgggcgcgccgacccagctttcttgtacaaagtggttgagggcctgaacgacatcttcgaggcccagaagatcgagtggcacgaggagaacctttactttcaaggtcaattggaaaatctctatttccagggaccaccagcgccaccacagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggattatgatattccaactactgcaagcgagaatttggcagcacagggtgagggtggtacgcgtaccggtcatcatcaccatcaccattgagtttaaactacgggctgcaggaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctagctagctagtcgagctcaacttcgaattcgatatcaagcttatcgcgataccgtcgacctcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgctcaagcttcgaattaattctgcagagctcggtacctttaagaccaatgacttacaaggcagctgtagatcttagccactttttaaaagaaaaggggggactggaagggctaattcactcccaacgaagacaagatgggatcaattcaccatgggaataacttcgtatagcatacattatacgaagttatgctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagt

SEQ ID NO: 69 - DNA, 12640 bp, Artificial Sequence
  Description of Artificial Sequence Synthetic polynucleotide
cagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatggtccgaggttctagacgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcaccggtcactagtccagtgtggtggaattctgcagatatcaacaagtttgtacaaaaaagcaggctccgcggccgcccccttcaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtctagaaagggtgggcgcgccgacccagctttcttgtacaaagtggttgagggcctgaacgacatcttcgaggcccagaagatcgagtggcacgaggagaacctttactttcaaggtcaattggaaaatctctatttccagggaccaccagcgccaccacagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggattatgatattccaactactgcaagcgagaatttggcagcacagggtgagggtggtacgcgtaccggtcatcatcaccatcaccattgagtttaaactacgggctgcaggaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctagctagctagtcgagctcaacttcgaattcgatatcaagcttatcgcgataccgtcgacctcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgctcaagcttcgaattaattctgcagagctcggtacctttaagaccaatgacttacaaggcagctgtagatcttagccactttttaaaagaaaaggggggactggaagggctaattcactcccaacgaagacaagatgggatcaattcaccatgggaataacttcgtatagcatacattatacgaagttatgctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagt

SEQ ID NO: 70 - DNA, 12031 bp, Artificial Sequence
  Description of Artificial Sequence Synthetic polynucleotide
cagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatggtccgaggttctagacgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcaccggtcactagtccagtgtggtggaattctgcagatatcaacaagtttgtacaaaaaagcaggctccgcggccgcccccttcaccatggagctgctgtgttgcgaaggcacccggcacgcgccccgggccgggccggacccgcggctgctgggggaccagcgtgtcctgcagagcctgctccgcctggaggagcgctacgtaccccgcgcctcctacttccagtgcgtgcagcgggagatcaagccgcacatgcggaagatgctggcttactggatgctggaggtatgtgaggagcagcgctgtgaggaggaagtcttccccctggccatgaactacctggatcgctacctgtcttgcgtccccacccgaaaggcgcagttgcagctcctgggtgcggtctgcatgctgctggcctccaagctgcgcgagaccacgcccctgaccatcgaaaaactgtgcatctacaccgaccacgctgtctctccccgccagttgcgggactgggaggtgctggtcctagggaagctcaagtgggacctggctgctgtgattgcacatgatttcctggccttcattctgcaccggctctctctgccccgtgaccgacaggccttggtcaaaaagcatgcccagacctttttggccctctgtgctacagattatacctttgccatgtacccgccatccatgatcgccacgggcagcattggggctgcagtgcaaggcctgggtgcctgctccatgtccggggatgagctcacagagctgctggcagggatcactggcactgaagtggactgcctgcgggcctgtcaggagcagatcgaagctgcactcagggagagcctcagggaagcctctcagaccagctccagcccagcgcccaaagccccccggggctccagcagccaagggcccagccagaccagcactcctacagatgtcacagccatacacctggccagcaagggcggcggcggcagcctggaggtgctgttccagggccccagccgcatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagaagggtgggcgcgccgacccagctttcttgtacaaagtggttgagggcctgaacgacatcttcgaggcccagaagatcgagtggcacgaggagaacctttactttcaaggtcaattggaaaatctctatttccagggaacgcgtaccggtcatcatcaccatcaccattgagtttaaactacgggctgcaggaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctagctagctagtcgagctcaacttcgaattcgatatcaagcttatcgcgataccgtcgacctcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgctcaagcttcgaattaattctgcagagctcggtacctttaagaccaatgacttacaaggcagctgtagatcttagccactttttaaaagaaaaggggggactggaagggctaattcactcccaacgaagacaagatgggatcaattcaccatgggaataacttcgtatagcatacattatacgaagttatgctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagt

SEQ ID NO: 71 - DNA, 11848 bp, Artificial Sequence
  Description of Artificial Sequence Synthetic polynucleotide
cagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatggtccgaggttctagacgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcaccggtcactagtccagtgtggtggaattctgcagatatcaacaagtttgtacaaaaaagcaggctccgcggccgcccccttcaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtctagaaagggtgggcgcgccgacccagctttcttgtacaaagtggttgagggcctgaacgacatcttcgaggcccagaagatcgagtggcacgaggagaacctttactttcaaggtcaattggaaaatctctatttccagggaacgcgtaccggtcatcatcaccatcaccattgagtttaaactacgggctgcaggaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctagctagctagtcgagctcaacttcgaattcgatatcaagcttatcgcgataccgtcgacctcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgctcaagcttcgaattaattctgcagagctcggtacctttaagaccaatgacttacaaggcagctgtagatcttagccactttttaaaagaaaaggggggactggaagggctaattcactcccaacgaagacaagatgggatcaattcaccatgggaataacttcgtatagcatacattatacgaagttatgctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagt

SEQ ID NO: 72 - DNA, 54 bp, Artificial Sequence
  Description of Artificial Sequence Synthetic oligonucleotide
gccagcaagggcggcggcggcagcctggaggtgctgttccagggccccagccgc

SEQ ID NO: 73 - protein, 18 aa, Artificial Sequence
  Description of Artificial Sequence Synthetic peptide
ASKGGGGSLEVLFQGPSR

SEQ ID NO: 74 - DNA, 4419 bp, Artificial Sequence
  Description of Artificial Sequence Synthetic polynucleotide
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcccccttcaccatggagctgctgtgttgcgaaggcacccggcacgcgccccgggccgggccggacccgcggctgctgggggaccagcgtgtcctgcagagcctgctccgcctggaggagcgctacgtaccccgcgcctcctacttccagtgcgtgcagcgggagatcaagccgcacatgcggaagatgctggcttactggatgctggaggtatgtgaggagcagcgctgtgaggaggaagtcttccccctggccatgaactacctggatcgctacctgtcttgcgtccccacccgaaaggcgcagttgcagctcctgggtgcggtctgcatgctgctggcctccaagctgcgcgagaccacgcccctgaccatcgaaaaactgtgcatctacaccgaccacgctgtctctccccgccagttgcgggactgggaggtgctggtcctagggaagctcaagtgggacctggctgctgtgattgcacatgatttcctggccttcattctgcaccggctctctctgccccgtgaccgacaggccttggtcaaaaagcatgcccagacctttttggccctctgtgctacagattatacctttgccatgtacccgccatccatgatcgccacgggcagcattggggctgcagtgcaaggcctgggtgcctgctccatgtccggggatgagctcacagagctgctggcagggatcactggcactgaagtggactgcctgcgggcctgtcaggagcagatcgaagctgcactcagggagagcctcagggaagcctctcagaccagctccagcccagcgcccaaagccccccggggctccagcagccaagggcccagccagaccagcactcctacagatgtcacagccatacacctggccagcaagggcggcggcggcagcctggaggtgctgttccagggccccagccgcatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagaagggtgggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 75 - DNA, 4419 bp, Artificial Sequence
  Description of Artificial Sequence Synthetic polynucleotide
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcccccttcaccatggagctgctgtgttgcgaaggcacccggcacgcgccccgggccgggccggacccgcggctgctgggggaccagcgtgtcctgcagagcctgctccgcctggaggagcgctacgtaccccgcgcctcctacttccagtgcgtgcagcgggagatcaagccgcacatgcggaagatgctggcttactggatgctggaggtatgtgaggagcagcgctgtgaggaggaagtcttccccctggccatgaactacctggatcgctacctgtcttgcgtccccacccgaaaggcgcagttgcagctcctgggtgcggtctgcatgctgctggcctccaagctgcgcgagaccacgcccctgaccatcgaaaaactgtgcatctacaccgaccacgctgtctctccccgccagttgcgggactgggaggtgctggtcctagggaagctcaagtgggacctggctgctgtgattgcacatgatttcctggccttcattctgcaccggctctctctgccccgtgaccgacaggccttggtcaaaaagcatgcccagacctttttggccctctgtgctacagattatacctttgccatgtacccgccatccatgatcgccacgggcagcattggggctgcagtgcaaggcctgggtgcctgctccatgtccggggatgagctcacagagctgctggcagggatcactggcactgaagtggactgcctgcgggcctgtcaggagcagatcgaagctgcactcagggagagcctcagggaagcctctcagaccagctccagcccagcgcccaaagccccccggggctccagcagccaagggcccagccagaccagcactcctacagatgtcacagccatacacctggccagcaagggcggcggcggcagcctggaggtgctgttccagggccccagccgcatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttgcacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagaagggtgggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 76 - DNA, 32 bp, Artificial Sequence
  Description of Artificial Sequence Synthetic oligonucleotide
acagctaccagatggcacttgcacccgtggtt

SEQ ID NO: 77 - DNA, 32 bp, Artificial Sequence
  Description of Artificial Sequence Synthetic oligonucleotide
aaccacgggtgcaagtgccatctggtagctgt

SEQ ID NO: 78 - DNA, 1836 bp, Artificial Sequence
  Description of Artificial Sequence Synthetic polynucleotide
atggagctgctgtgttgcgaaggcacccggcacgcgccccgggccgggccggacccgcggctgctgggggaccagcgtgtcctgcagagcctgctccgcctggaggagcgctacgtaccccgcgcctcctacttccagtgcgtgcagcgggagatcaagccgcacatgcggaagatgctggcttactggatgctggaggtatgtgaggagcagcgctgtgaggaggaagtcttccccctggccatgaactacctggatcgctacctgtcttgcgtccccacccgaaaggcgcagttgcagctcctgggtgcggtctgcatgctgctggcctccaagctgcgcgagaccacgcccctgaccatcgaaaaactgtgcatctacaccgaccacgctgtctctccccgccagttgcgggactgggaggtgctggtcctagggaagctcaagtgggacctggctgctgtgattgcacatgatttcctggccttcattctgcaccggctctctctgccccgtgaccgacaggccttggtcaaaaagcatgcccagacctttttggccctctgtgctacagattatacctttgccatgtacccgccatccatgatcgccacgggcagcattggggctgcagtgcaaggcctgggtgcctgctccatgtccggggatgagctcacagagctgctggcagggatcactggcactgaagtggactgcctgcgggcctgtcaggagcagatcgaagctgcactcagggagagcctcagggaagcctctcagaccagctccagcccagcgcccaaagccccccggggctccagcagccaagggcccagccagaccagcactcctacagatgtcacagccatacacctggccagcaagggcggcggcggcagcctggaggtgctgttccagcccagccgcatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggag

SEQ ID NO: 79 - protein, 612 aa, Artificial Sequence; identical to SEQ ID NO: 10
  Description of Artificial Sequence Synthetic polypeptide

SEQ ID NO: 80 - DNA, 1848 bp, Artificial Sequence
  Description of Artificial Sequence Synthetic polynucleotide
atggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatcgccagcaagggcggcggcggcagcctggaggtgctgttccagggccccagccgcatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggag

SEQ ID NO: 81 - protein, 616 aa, Artificial Sequence
  Description of Artificial Sequence Synthetic polypeptide
MEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDNSIRPEELLQMELLLVNKLKWNLAAMTPHDFIEHFLSKMPEAEENKQIIRKHAQTFVALCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACTPTDVRDVDIASKGGGGSLEVLFQGPSRMATSRYEPVAEIGVGAYGTVYKARDPHSGHFVALKSVRVPNGGGGGGGLPISTVREVALLRRLEAFEHPNVVRLMDVCATSRTDREIKVTLVFEHVDQDLRTYLDKAPPPGLPAETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFPPRGPRPVQSVVPEMEESGAQLLLEMLTFNPHKRISAFRALQHSYLHKDEGNPE

SEQ ID NO: 82 - DNA, 4428 bp, Artificial Sequence
  Description of Artificial Sequence Synthetic polynucleotide
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcccccttcaccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatcgccagcaagggcggcggcggcagcctggaggtgctgttccagggccccagccgcatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagaagggtgggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 83 - DNA, 26 bp, Artificial Sequence
  Description of Artificial Sequence Synthetic primer
gtacagagtgatattattgacacgcc

SEQ ID NO: 84 - DNA, 29 bp, Artificial Sequence
  Description of Artificial Sequence Synthetic primer
tcaaccactttgtacaagaaagctgaacg

SEQ ID NO: 85 - DNA, 40 bp, Artificial Sequence
  Description of Artificial Sequence Synthetic primer
tacaaagtggttgagggcctgaacgacatcttcgaggccc

SEQ ID NO: 86 - DNA, 36 bp, Artificial Sequence
  Description of Artificial Sequence Synthetic primer
ctcgtgccactcgatcttctgggcctcgaagatgtc

SEQ ID NO: 87 - DNA, 36 bp, Artificial Sequence
  Description of Artificial Sequence Synthetic primer
gatcgagtggcacgaggagaacctttactttcaagg

SEQ ID NO: 88 - DNA, 40 bp, Artificial Sequence
  Description of Artificial Sequence Synthetic primer
atgatgaccggtacgcgtaccaccctcaccctgtgctgcc

SEQ ID NO: 89 - DNA, 40 bp, Artificial Sequence
  Description of Artificial Sequence Synthetic primer
atgatgaccggtacgcgttccctggaaatagagattttcc

(19) United States
(12) Patent Application Publication
(10) Pub. No.: US 2015/0299764 A1
(43) Pub. Date: Oct. 22, 2015

(54) IMMOBILISED CYCLIN-DEPENDENT KINASE 4 FUSION PROTEINS AND USES THEREOF

(71) Applicant: UNIVERSITE LIBRE DE BRUXELLES, Bruxelles (BE)

(72) Inventors: Eric Raspe, Mouscron (BE); Pierre Roger, Bruxelles (BE); Katia Coulonval, Enghien (BE); Sabine Paternot, Hoves (BE); Xavier Bisteau, Bruxelles (BE)

(21) Appl. No.: 14/425,380

(22) PCT Filed: Sep. 5, 2013
(86) PCT No.: PCT/EP2013/068399

§ 371 Date: Mar. 3, 2015

(30) Foreign Application Priority Data
Sep. 5, 2012 (EP) PCT/EP2012/067343

(51) Int. Cl. C12Q 1/48

(52) U.S. Cl. CPC C12Q 1/485

(57) ABSTRACT

The present invention concerns an in vitro assay for determining the activation status of endogenous CDK4 in eukaryotic cells, said assay comprising the steps of: providing eukaryotic cells maintained in a quiescent state, said eukaryotic cells comprising a cyclin D/CDK4 fusion protein, wherein the CDK4 part of the fusion protein is present in a hypophosphorylated form; inducing proliferation of said eukaryotic cells; isolating the cyclin D/CDK4 fusion protein from said eukaryotic cells; and measuring the activation status of said isolated cyclin D/CDK4 fusion protein, thereby determining the activation stats of endogenous CDK4 in said eukaryotic cells.

### FIELD OF THE INVENTION

The present invention provides cyclin-dependent kinase 4 fusion proteins and uses thereof, especially assays for determining the activation status of endogenous cyclin-dependent kinase 4 (CDK4).

### BACKGROUND OF THE INVENTION

CDK4 acts as a master integrator in the G1 phase, coupling with the cell cycle mitogenic and antimitogenic signals as well as with their oncogenic counterparts in cancer cells. CDK4 phosphorylates and inactivates the cell cycle/tumor suppressor proteins of the retinoblastoma (Rb) family (p105Rb, p107, and p130Rb2). This leads to both E2F-dependent transcription of essential cell cycle enzymes and regulators and assembly of the pre-replication complex.

The activation of CDK4 is a multistep process that requires the binding of a D-type cyclin (D1, D2, or D3) and an activating phosphorylation in the T-loop at threonine 172 (Thr172 or T172) for CDK4. At variance with the coexistence of several CDK-activating kinases (CAK) in fungi and plants, in animal cells one single CAK complex is considered to be responsible for activating phosphorylation of the various cell cycle CDKs, including CDK4. This CAK complex, constituting of CDK7, cyclin H, and Mat 1 is constitutively active during the cell cycle and is not regulated by external mitogenic stimulations. However, the activating phosphorylation of CDK4 on Thr172 is not constitutive, contrary to the analogous phosphorylation on CDK2 and CDK1, but is extremely regulated (Paternot et al., 2010, Cell cycle, 9, 689-699). This contradiction has led to the hypothesis that the constitutively active CAK/CDK7 complex is not or is not only responsible for the phosphorylation of CDK4.

To date, the regulation of the phosphorylation of CDK4 for example by other CAK complexes has been very poorly studied because only few biochemical techniques are available to study this regulation and moreover, the available ones are tedious.

For example, the most direct way to evaluate if the CAK/CDK7 complex directly phosphorylates CDK4 is to inhibit the complex's activity and to analyze the impact on the phosphorylation of CDK4. Unfortunately, there is no specific inhibitor for CDK7.

Another possibility to study the phosphorylation of Thr172 involves the use of sodium dodecyl sulfate-polyacrylamide gel electrophoresis (SDS-PAGE). However, the phosphorylation on Thr172 of CDK4 does not affect the one-dimensional migration of the protein. Two-dimensional electrophoresis after immunoprecipitation of CDK4 allows to separate the phosphorylated form based on the modification of electric charge. However, this technique is laborious and requires large amounts of cells and reagents. Hence, this technique is incompatible with high throughput analysis envisaged to identify novel CDK activating kinases or ways to interfere with CDK4 activation.

In view of the above, it is an object of the present invention to provide an assay which allows to study the regulation of the phosphorylation of CDK4. Furthermore, it is an object of the present invention to provide assays which allow easy screening of novel CDK activating kinases and inhibitors thereof. Finally, it is an object of the present invention to provide a tool to generate and characterize antibodies to be used to directly detect CDK4 activation via phosphorylation.

### SUMMARY OF THE INVENTION

The present invention provides new methods, tools, and assays addressing one or more of the above-mentioned problems of the prior art.

The present invention provides an in vitro assay for determining the activation status of endogenous cyclin-dependent kinase 4 (CDK4) in eukaryotic cells, said assay comprising the steps of:
providing eukaryotic cells maintained in a quiescent state, said eukaryotic cells comprising a cyclin D/CDK4 fusion protein, wherein the CDK4 part of the fusion protein is present in a hypophosphorylated form;
inducing proliferation of said eukaryotic cells;
isolating the cyclin D/CDK4 fusion protein from said eukaryotic cells; and
measuring the activation status of said isolated cyclin D/CDK4 fusion protein, thereby determining the activation status of endogenous CDK4 in said eukaryotic cells.

In this assay, the regulation of the phosphorylation of the cyclin D/CDK4 fusion protein happens in the eukaryotic cells.

In another aspect, the present invention relates to an in vitro assay for determining the activation status of endogenous cyclin-dependent kinase 4 (CDK4) in a eukaryotic cell extract, said assay comprising the steps of:
providing a cyclin D/CDK4 fusion protein, wherein the CDK4 part of the fusion protein is present in a hypophosphorylated form;
contacting the cyclin D/CDK4 fusion protein with a eukaryotic cell extract; and
measuring the activation status of said isolated cyclin D/CDK4 fusion protein, thereby determining the activation status of endogenous CDK4 in said eukaryotic cell extract.

In this assay, the regulation of the phosphorylation the cyclin D/CDK4 fusion protein happens outside the cells.

The assays according to the present invention advantageously allow to study the mechanisms involved in the regulation of the activation of endogenous CDK4 for instance during cell cycle progression, in tumor cell lines, in tumors, etc. Moreover, such assays advantageously allow studying the regulation of the activation of endogenous CDK4 independently of the expression of cyclin D and/or the assembly of D-type cyclins with CDK4.

The present inventors have unexpectedly found that the assays embodying the principles of the present invention advantageously allow high throughput screening for activating kinases of CDK4 in an efficient and inexpensive way. The assays of the invention allow studying the upstream signaling cascade of endogenous CDK4. For instance, the assays of the present invention allow high throughput screening of siRNA libraries of the kinome to identify kinases involved in the activation of endogenous CDK4.

Additionally, the assays of the present invention also allow high throughput screening of compounds able to activate or inactivate endogenous CDK4 activity. Since there is considerable evidence that the deregulation of cyclins and CDKs, in particular those controlling G1 progression may be involved in the development of many human cancers (Hall and Peters, 1996, Adv. Cancer Res. 68, 67-108; Sellers and Kaelin, 1997, J. Clin. Oncol., 15, 3301-3312; Sherr, 1996, Science, 274, 1672-1677) and in the development of proliferative diseases such as restenosis or psoriasis, screening of inhibitory compounds of endogenous CDK4 activity is of major importance.

In a further aspect, the present invention provides the use of a reporter molecule for determining the activation status of endogenous CDK4, wherein the reporter molecule comprises a cyclin D/cyclin-dependent-kinase 4 (CDK4) fusion protein. The use according to the present invention advantageously allows studying the mechanisms involved in the regulation of the activation of endogenous CDK4. Preferably, said reporter molecule also comprises a tag, which can be used to isolate the reporter molecule from e.g. a cell extract.

In a further aspect, the present invention provides a reporter molecule for determining the activation status of endogenous CDK4, wherein the reporter molecule comprises a cyclin D/CDK4 fusion protein and an Avi tag. Surprisingly, the inventors found that the Avi tag does not interfere with the function of the cyclin D/CDK4 fusion protein.

In a further aspect, the present invention provides a reporter system comprising the reporter molecule as taught above and the biotin ligase BirA. Such a reporter system advantageously allows purification of the reporter molecule, while retaining the functionality of the cyclin D/CDK4 fusion protein.

In a further aspect, the present invention provides a kit for determining the activation status of endogenous CDK4 in eukaryotic cells or a eukaryotic cell extract, said kit comprising a eukaryotic cell line and a cyclin D/CDK4 fusion protein.

In a further aspect, the present invention provides a kit for determining the activation status of endogenous CDK4 in eukaryotic cells or a eukaryotic cell extract, said kit comprising a eukaryotic cell line and a nucleic acid encoding a cyclin D/CDK4 fusion protein.

In a further aspect, the present invention provides an assay for generating phospho-specific CDK4 binding molecules such as antibodies or fragments thereof, nanobodies or aptamers that specifically recognize the phosphorylated form of CDK4.

In a further aspect, the present invention provides a tool for determining whether such a binding molecule, such as an antibody, a nanobody or an aptamer specifically recognizes the phosphorylated form of CDK4.

The invention further provides for the use of the cyclin D/CDK4 fusion tandemly purified from serum-stimulated eukaryotic cells as immunogen to create phospho CDK4-specific binding molecules.

A typical assay to identify and characterize phospho CDK4-specific binding molecules comprises the steps of:
providing eukaryotic cells maintained in a quiescent state, said eukaryotic cells comprising a cyclin D/CDK4 fusion protein, wherein the CDK4 part of the fusion protein is present in a hypophosphorylated form;
providing eukaryotic cells maintained in a serum stimulated state, said eukaryotic cells comprising a cyclin D/CDK4 fusion protein, wherein the CDK4 part of the fusion protein is present in a hyperphosphorylated form;
isolating the cyclin D/CDK4 fusion protein from said eukaryotic cells;
immobilizing the cyclin D/CDK4 fusion protein from said quiescent or serum-stimulated eukaryotic cells on a suitable matrix;
contacting the immobilized cyclin D/CDK4 fusion protein from said quiescent or serum-stimulated eukaryotic cells with a candidate binding molecule or binding molecule preparation;
comparing the binding of said a candidate binding molecule or binding molecule preparation to the immobilized cyclin D/CDK4 fusion protein from serum-stimulated eukaryotic cells with the binding of said a candidate binding molecule or binding molecule preparation to the immobilized cyclin D/CDK4 fusion protein from quiescent eukaryotic cells;
selecting the binding molecule or binding molecule preparation that preferably binds to the immobilized cyclin D/CDK4 fusion protein from serum-stimulated eukaryotic cells versus quiescent eukaryotic cells, thereby obtaining a phospho CDK4-specific binding molecule or binding molecule preparation.

Preferably, said binding agent is selected from the group comprising: a specific antibody, antigen-binding antibody fragment, nanobody, affybody, an aptamer, a photoaptamer, a spiegelmer, a small molecule, an interacting partner, a specifically binding protein or peptide, a Darpin, an ankyrin, an isotopically labelled tracer or a ligand.

Preferably, said suitable matrix is composed of a molecule coupled to a suitable support, said molecule being adapted to the used affinity purification tag fused to the cyclin D/CDK4 fusion protein. Preferably, said suitable support is selected from the group comprising: agarose, sepharose, polystyrene, polyethylene, gold (Biacore), 0.8 micron-sized iron, super-paramagnetic, hydrophobic, and polymer-encapsulated (non-exposed iron) beads (Nanolink, . . . ), etc. When the biotinylated Avi-tag is used, said suitable molecule is selected from the group comprising: avidin, streptavidin, neutravidin, CaptAvidin, Tamavidin, etc. When GST is used, said suitable molecule is glutathione.

When MBP is used, said suitable molecule is amylose. When His-tag is used, said suitable molecules are Ni chelates. When Halo-tag is used, said suitable molecules are chloroalkane linkers attached to a variety of useful molecules, such as affinity handles, or solid surfaces. When Snap/Clip-tag is used, said suitable molecules are O⁶-benzylguanine or O²-benzylcytosine derivatives.

These and further aspects and embodiments of the invention are hereunder further explained in the following sections and in the claims, and illustrated by non-limiting figures.

### BRIEF DESCRIPTION OF THE FIGURES

FIG. 1 schematically represents a reporter system according to an embodiment of the present invention comprising a reporter molecule according to an embodiment of the present invention and the biotine ligase (BirA) protein, and there under a construct comprising a nucleic acid sequence encoding a reporter system according to an embodiment of the present invention. TET: tetracyclin repressor; cyclin D/CDK4 fusion: cyclin D/CDK4 fusion protein; Avi: Avi tag; TEV: tobacco etch virus cleavage site; EGFP: enhanced green fluorescent protein; His: poly-His purification tag; STOP: stop codon; IRES: internal ribosome entry site; BirA: bifunctional protein biotine ligase BirA.

FIG. 2 schematically represents reporter systems according to different embodiments of the present invention. CycD-CDK4: cyclin D/CDK4 fusion protein; Avi: Avi tag; TEV: tobacco etch virus cleavage site; EGFP: enhanced green fluorescent protein; His: poly-His purification tag; BirA: bifunctional protein biotine ligase BirA; GST: gluthathion-S-transferase; Luc: luciferase.

FIG. 3 schematically represents different embodiments of an assay according to the present invention.

FIG. 4 schematically represents two embodiments of an assay according to the present invention.

FIG. 5 represents a graph plotting the percentage of cell nuclei labelled with bromodeoxyuridine as a function of time. The graph illustrates the DNA synthesis for control cells rendered quiescent (dashed line) and cells stimulated at time point zero with 5% serum and 6 ng/ml insulin (full line).

FIG. 6 represents immunoblots illustrating cyclin protein expression levels and Rb phosphorylation as a function of time. Rb phosphorylation is recognized by the upward shift of the apparent molecular weight of the Rb protein detected with a mix of anti-Rb and anti-phospho-RB antibodies. Cont: control cells rendered quiescent; Serum: cells stimulated at time point zero with 5% serum and 6 ng/ml insulin; RB: retinoblastoma protein; RB-P: phosphorylated retinoblastoma protein; CycD1: cyclin D1; CycD3: cyclin D3; CycA: cyclin A; CycB: cyclin B; and CycE: cyclin E.

FIG. 7A represents 2D immunoblots illustrating endogenous CDK4 abundance after immunoprecipitation with cyclin D1 (IP D1) or cyclin D3 (IP D3) in control cells rendered quiescent (Cont 8 h) or 8 h after stimulation of quiescent cells with 5% serum and 6 ng/ml insulin (Serum 8 h).

FIG. 7B represents an immunoblot illustrating the Rb kinase activity of the immunoprecipitate determined by the phosphorylation status of a Rb fragment (Rb-P) after its incubation with the immunoprecipitate in the presence of ATP and in control cells rendered quiescent (indicated −) or after stimulation of quiescent cells with 5% serum and 6 ng/ml insulin (indicated +) for the indicated time periods.

FIG. 8 represents immunoblots illustrating the expression of the cyclin D/CDK4 fusion proteins detected with anti-CDK4, anti-cyclin D1 or anti-cyclin D3, and illustrating the biotinylation status (Streptavidin-HRP) of HEK293T cells after transient transfection of the HEK293T cells with 1: pBluescript, 2: pWFAvi_IBc_D1 (D1+Bc), 3: empty vector (Empty), 4: pWFAvi_IBc_D3L2K4 (D3L2K4+Bc), 5: pWFAvi_IBp_D1 (D1+Bp), 6: pWFAvi_IBp_D1L1K4 (D1L1K4+Bp), 7: pWFAvi_IBp_D3L2K4 (D3L2K4+Bp).

FIG. 9A represents a graph plotting the input luciferase activity and the proportion of luciferase activity bound to streptavidin coated sepharose beads after purification of cell extracts of HEK293T cells transiently transfected with 1: pBluescript; 2: pWFAvi_IBc_D3L2K4 (D3L2K4+Bc); 3: pWFAvi_IBp_D3L2K4 (D3L2K4+Bp); 4: pWFAvi_IBp_D1L1K4 (D1L1K4+Bp); 5: pWFAvi_IBc_luc (Luc+Bc).

FIG. 9B represents immunoblots illustrating the expression of the cyclin D3/CDK4 fusion with the linker 2 (noted D3L2K4) and its Rb kinase activity determined by the phosphorylation status of Rb (Rb-P) in cell extracts of HEK293T cells after transient transfection of HEK293T cells with 1: pBluescript (control=CT); 2: pWFAvi_IBc_D3L2K4 (D3L2K4+Bc); 3: pWFAvi_IBp_D3L2K4 (D3L2K4+Bp); 4: pWFAvi_IBp_D1L1K4 (D1L1K4+Bp); 5: pWFAvi_IBc_luc (Luc+Bc).

FIG. 10 represents immunoblots illustrating the expression of luciferase fused to the EGFP and the Avi-tag (luc), the expression of the tetracycline repressor (Tet), the expression of the endogeneous CDK4 (Endogeneous CDK4), the expression of the cyclin D3/CDK4 fusion with the linker 2 detected with the anti-CDK4 antibody (CDK4), the anti-cyclin D3 antibody, or the expression of EGFP with the anti-EGFP antibody (EGFP) as well as its biotinylation status (biotinylation) in cell extracts of MCF7 or MCF7KR cells after their infection with lentiviruses produced with the plasmids 1: pWFAvi_IBc_luc (Luc+Bc); 2, 4, 5: pWFAvi_IBc_D3L2K4 (D3L2K4+Bc); 3, 6, 7: pWFAvi_IBp_D3L2K4 (D3L2K4+Bp); stimulated (5 and 7) or not (1, 2, 3, 4, 6) with doxycyclin compared to cell extracts of parental non-infected MCF7 cells (MCF7 CT).

FIG. 11A represents a graph plotting the input luciferase activity and the proportion of luciferase activity bound to streptavidin coated sepharose beads after purification of cell extracts of MCF7 cells infected with lentiviruses produced with vectors allowing the expression of untagged non-biotinylable luciferase, Avi-tagged biotinylable luciferase, or Avi-tagged biotinylable cyclin D3/CDK4 fusion. MCF7 cells were rendered quiescent, and kept quiescent (indicated −) or stimulated at time point zero with 5% serum and 6 ng/ml insulin (indicated +), and allowed to proliferate for 16 h in the presence of biotin before the preparation of cell extracts. 1: MCF7 unexposed to serum; 2: MCF7 exposed to serum; 3: MCF7 expressing a non biotinylable luciferase unexposed to serum; 4: MCF7 expressing a non biotinylable luciferase exposed to serum; 5: MCF7 expressing a biotinylable avi-tagged luciferase (LucAvi) together with the BirA/mCherry fusion (Bc) unexposed to serum; 6: MCF7 expressing a biotinylable avi-tagged luciferase (LucAvi) together with the BirA/mCherry fusion (Bc) exposed to serum; 7: MCF7 expressing the biotinylable avi-tagged cyclin D3/CDK4 fusion with the linker 2 D3L2K4) with the BirA/mCherry fusion (Bc) unexposed to serum; 8: MCF7 expressing the biotinylable avi-tagged cyclin D3/CDK4 fusion with the linker 2 D3L2K4) with the BirA/mCherry fusion (Bc) exposed to serum.

FIG. 11B represents immunoblots illustrating the expression of the cyclin D3/CDK4 fusion with the linker 2 (noted D3L2K4) detected with the anti-CDK4 antibody, the endogeneous CDK4 expression (endogenous CDK4) and the kinase activity determined by the phosphorylation status of a Rb substrate detected with an anti-phospho-Rb antibody (Rb-P) in cell extracts of MCF7 cells infected with lentiviruses produced with vectors allowing the expression of untagged non-biotinylable luciferase, Avi-tagged biotinylable luciferase, or Avi-tagged biotinylable cyclin D3/CDK4 fusion. 1: MCF7 unexposed to serum; 2: MCF7 exposed to serum; 3: MCF7 expressing a non biotinylable luciferase unexposed to serum; 4: MCF7 expressing a non biotinylable luciferase exposed to serum; 5: MCF7 expressing a biotinylable avi-tagged luciferase (LucAvi) together with the BirA/mCherry fusion (Bc) unexposed to serum; 6: MCF7 expressing a biotinylable avi-tagged luciferase (LucAvi) together with the BirA/mCherry fusion (Bc) exposed to serum; 7: MCF7 expressing the biotinylable avi-tagged cyclin D3/CDK4 fusion with the linker 2 D3L2K4) with the BirA/mCherry fusion (Bc) unexposed to serum; 8: MCF7 expressing the biotinylable avi-tagged cyclin D3/CDK4 fusion with the linker 2 D3L2K4) with the BirA/mCherry fusion (Bc) exposed to serum.

FIG. 12 represents immunoblots illustrating the expression of the cyclin D3/CDK4 fusion with the linker 2 (noted fusion D3K4) detected with the anti-CDK4 antibody, the endogeneous CDK4 expression (noted CDK4) and the kinase activity determined by the phosphorylation status of a Rb substrate detected with an anti-phospho-Rb antibody (noted P-Rb826) in cell extracts of HCT116K7AS cells infected with lentiviruses produced with vectors allowing the expression of Avi-tagged biotinylable cyclin D3/CDK4 fusion together with the BirA/mCherry fusion hereafter named HCT116K7AS-V26. 1: HCT116K7AS-V26 unexposed to serum; 2: HCT116K7AS exposed to serum and DMSO used as vehicle for 5 hours; 3: HCT116K7AS-V26 exposed to serum and 1-NMPP1 (noted NMPP1) to block the modified NMPP1-inhibitable CDK7 expressed in the HCT116K7AS cells for 5 hours; 4: HCT116K7AS-V26 exposed to serum for 5 hours and DMSO used as vehicle for one additional hour; 5: HCT116K7AS-V26 exposed to serum for 5 hours and 1-NMPP1 for one additional hour; 6: HCT116K7AS-V26 exposed to DMSO for one hour; 7: HCT116K7AS-V26 exposed to serum for 16 hours and DMSO used as vehicle for one additional hour; 8: HCT116K7AS-V26 exposed to serum for 16 hours and 1-NMPP1 for one additional hour.

FIG. 13 represents immunoblots illustrating the expression of the luciferase or of the Avi-tagged luciferase/EGFP fusion detected with the anti-luciferase antibody (luc), the expression of the BirA biotin ligase detected with a anti-BirA antibody (BirA), the expression of the Avi-tagged cyclin D3/CDK4/EGFP fusions with the linker 2 or the linker 3 detected with the anti-EGFP antibody (EGFP) as well as their biotinylation status (streptavidin) in cell extracts of HEK293T cells transfected with vectors allowing the expression of Avi-tagged biotinylable cyclin D3/CDK4 fusion together with the BirA/mCherry fusion 0: no transfection; 1: non biotinylable luciferase (Luc), 2: biotinylable luciferase coupled to the BirA/mCherry fusion (AviLuc+BC), 3: wild type biotinylable D3/CDK4/EGFP fusion with the linker 2 coupled to the BirA/mCherry fusion (AviD3L2K4+Bc), 4: wild type biotinylable D3/CDK4/EGFP fusion with the linker 3 coupled to the BirA/mCherry fusion (AviD3L3K4+Bc), 5: non-activable mutant of the biotinylable D3/CDK4/EGFP fusion with the linker 3 coupled to the BirA/mCherry fusion (AviD3L3K4T172A+Bc).

FIG. 14 represents immunoblots illustrating the expression of the Avi-tagged cyclin D3/CDK4/EGFP fusions with the linker 2 or the linker 3 detected with the anti-CDK4 (noted CDK4) or the anti-CCND3 (noted CCND3) antibodies and the Rb-kinase activity determined by the phosphorylation status of a Rb substrate detected with an anti-phospho-Rb antibody (noted Rb826) and the shift in molecular weight observed upon phosphorylation of the Rb substrate detected anti-GST antibody (noted GST) in cell extracts of HEK293T cells transfected with vectors allowing the expression of Avi-tagged biotinylable cyclin D3/CDK4 fusion together with the BirA/mCherry fusion 0: no transfection; 1: non biotinylable luciferase (Luc), 2: biotinylable luciferase coupled to the BirA/mCherry fusion (AviLuc+BC), 3: wild type biotinylable D3/CDK4/EGFP fusion with the linker 2 coupled to the BirA/mCherry fusion (AviD3L2K4+Bc), 4: wild type biotinylable D3/CDK4/EGFP fusion with the linker 3 coupled to the BirA/mCherry fusion (AviD3L3K4+Bc), 5: non-activable mutant of the biotinylable D3/CDK4/EGFP fusion with the linker 3 coupled to the BirA/mCherry fusion (AviD3L3K4T172A+Bc).

FIG. 15 represents immunoblots illustrating the bi-dimensional separation of native or phosphorylated Avi-tagged cyclin D3/CDK4/EGFP fusions with the linker 3 digested with TEV and Prescission proteases detected with the anti-CDK4 (noted CDK4) or the anti-Phospho-CDK4 (noted PT172) antibodies in cell extracts of HEK293T cells transfected with vectors allowing the expression of the wild type Avi-tagged biotinylable cyclin D3/CDK4/EGFP fusion together with the BirA/mCherry fusion.

FIG. 16 represents immunoblots illustrating the expression (noted CDK4 and Luciferase), biotinylation (noted streptavidi) and Rb-kinase activity (noted P-Rb826) of the wild-type or T172A mutated Avi-tagged biotinylable cyclin D3/CDK4 fusion with the linker 3 compared to Avi-tagged biotinylable luciferase, or Avi-tagged biotinylable cyclin D3/CDK4 fusion with the linker 2 in cell extracts of MCF7 cells infected with lentiviruses produced with vectors allowing the expression of the corresponding transgenes.

1: MCF7 expressing a biotinylable avi-tagged luciferase (noted Luc) together with the BirA/mCherry fusion; 2: MCF7 expressing a biotinylable avi-tagged cyclin D3/CDK4 fusion with the linker 2 (noted D3L2K4) with the BirA/mCherry fusion; 3: MCF7 expressing a wild type biotinylable avi-tagged cyclin D3/CDK4 fusion with the linker 3 (noted D3L3K4) with the BirA/mCherry fusion; 4: MCF7 expressing a T172A-mutated biotinylable avi-tagged cyclin D3/CDK4 fusion with the linker 3 (noted D3L3K4T172A) with the BirA/mCherry fusion.

FIG. 17 represents immunoblots illustrating the expression of the internally Avi-tagged cyclin D3/CDK4/Avi/EGFP or Luciferase/Avi/fusions coupled or not to GFP detected with the anti-BirA (noted BirA), anti-GFP (noted GFP), anti-luciferase (noted Luciferase), the anti-CDK4 (noted CDK4) or the anti-CCND3 (noted CCND3) antibodies or with labeled streptavidin (noted streptavidin) in cell extracts of HEK293T cells transfected with vectors allowing the expression of internally Avi-tagged biotinylable cyclin D3/CDK4 or Luciferase/Avi/GFP fusions and the expression of the BirA biotin ligase as well as the Rb-kinase activity of the corresponding cell extracts (noted Rb-P Thr826) determined by the phosphorylation status of a Rb substrate detected with an anti-phospho-Rb antibody 1: wild type biotinylable cyclin D3/CDK4/Avi/GFP fusion coexpressed with BirA (noted ATGHis_D3L3K4), 2: biotinylable luciferase/Avi/GFP fusion coexpressed with BirA (noted ATGHis_Luc), 3: wild type biotinylable cyclin D3/CDK4/Avi fusion coexpressed with BirA (noted ATHis_D3L3K4), 4: biotinylable luciferase/Avi fusion coexpressed with BirA (noted ATHis_Luc), 5: non biotinylable luciferase (Luc), 6: wild type biotinylable cyclin D3/CDK4/EGFP fusion with the linker 3 coupled to the BirA/mCherry fusion (Avi_IBc_D3L2K4); 7: biotinylable luciferase coupled to the BirA/mCherry fusion (Avi_IBc_Luc).

FIG. 18 represents immunoblots illustrating the Rb kinase activity determined by the phosphorylation status of a Rb substrate detected with an anti-phospho-Rb antibody in cell extracts obtained by the lysis with 6 different buffers together or not with glass/glass homogeneisation and/or sonication of HCT116K7AS cells infected with lentiviruses produced with vectors allowing the expression of Avi-tagged biotinylable cyclin D3/CDK4 fusion together with the BirA/mCherry fusion hereafter named HCT116K7AS-V26 unexposed to serum (noted Cont) or exposed to serum for 16 h (noted Serum 16 h). The buffers are presented in Example 22, Table 6.

FIG. 19 represents dot-blot images or quantification (noted Green/Red ratio) illustrating the Rb kinase activity determined by the phosphorylation status of a Rb substrate detected with an anti-phospho-Rb antibody in cell extracts of HCT116K7AS cells infected with lentiviruses produced with vectors allowing the expression of Avi-tagged biotinylable cyclin D3/CDK4 fusion together with the BirA/mCherry fusion hereafter named HCT116K7AS-V26 unexposed to serum (noted Cont); exposed to serum for 16 h (noted Serum 16 h); or exposed to serum and 1-NMPP1 for 16 h (noted Serum 16 h+NMPP1).

FIG. 20 compares the luciferase activity immobilized on the indicated matrices of dilutions in the indicated proportions of cell extracts of HEK293T cells transfected with vectors allowing the expression of Avi-tagged biotinylable luciferase together with the BirA/mCherry fusion.

FIG. 21 compares the Rb kinase activity (noted detection P-Rb826) immobilized on streptavidin-coated sepharose beads of cell extracts of HEK293T cells transfected with vectors allowing the expression of internally Avi-tagged biotinylable cyclin D3/CDK4/EGFP (noted ATGHis D3L3K4 wt) or Luciferase/EGFP (noted ATGHis Luc) fusions or the corresponding constructs lacking the EGFP sequence between the TEV cleavage sites and the poly-histidine tag (noted ATHis D3L3K4 and ATHis Luc, respectively) and the expression of the BirA biotin ligase determined by the phosphorylation status of a Rb substrate immobilized on gluthation-coated plates detected by the DELFIA assay with an anti-phospho-Rb primary antibody and an Eu-coupled anti-mouse secondary antibody, the amount of cyclin D3/CDK4 fusion from the corresponding extracts immobilized on the streptavidin-coated sepharose beads (noted detection CCND3) detected by the Delfia assay with an anti-CCND3 primary antibody and an Eu-coupled anti-mouse secondary antibody. The Rb-kinase activity of the corresponding cell extracts (noted Rb-P Thr826) determined by western blot showing the phosphorylation status of a Rb substrate detected with an anti-phospho-Rb antibody is displayed in the insert.

1: wild type biotinylable cyclin D3/CDK4/Avi/GFP fusion coexpressed with BirA (noted ATGHis_D3L3K4), 2: biotinylable luciferase/Avi/GFP fusion coexpressed with BirA (noted ATGHis_Luc), 3: wild type biotinylable cyclin D3/CDK4/Avi fusion coexpressed with BirA (noted ATHis_D3L3K4), 4: biotinylable luciferase/Avi fusion coexpressed with BirA (noted ATHis_Luc), 5: non biotinylable luciferase (Luc), 6: wild type biotinylable cyclin D3/CDK4/EGFP fusion with the linker 3 coupled to the BirA/mCherry fusion (Avi_IBc_D3L2K4); 7: biotinylable luciferase coupled to the BirA/mCherry fusion (Avi_IBc_Luc).

FIG. 22 compares the Rb kinase activity (noted detection P-Rb) immobilized on streptavidin-coated plates of cell extracts of MCF7 cells infected with lentiviruses produced with vectors allowing the expression of wild type internally Avi-tagged biotinylable cyclin D3/CDK4/EGFP or internally Avi-tagged biotinylable Luciferase/EGFP fusions expressed together with BirA and the rTTA3 regulator in the presence of the inducer doxycyclin and serum cultivated in 96 well plates determined by the phosphorylation status of a Rb substrate detected by the Delfia assay with an anti-phospho-Rb primary antibody and an Eu-coupled anti-mouse secondary antibody, to the amount of cyclin D3/CDK4 fusion from the corresponding extracts immobilized on the streptavidin-coated plates (noted detection CCND3) detected by the Delfia assay with an anti-CCND3 primary antibody and an Eu-coupled anti-mouse secondary.

### DETAILED DESCRIPTION OF THE INVENTION

Unless defined otherwise, all technical and scientific terms used herein have the same meaning as is commonly understood by one of skill in the art. All publications referenced herein are incorporated by reference thereto.

The articles ‘a’ and ‘an’ are used herein to refer to one or to more than one, i.e. to at least one of the grammatical object of the article.

Throughout this application, the term ‘about’ is used to indicate that a value includes the standard deviation of error for the device or method being employed to determine the value.

The recitation of numerical ranges by endpoints includes all integer numbers and, where appropriate, fractions subsumed within that range (e.g. 1 to 5 can include 1, 2, 3, 4 when referring to, for example, a number of elements). The recitation of end points also includes the end point values themselves (e.g. from 1.0 to 5.0 includes both 1.0 and 5.0).

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The present inventors have realised the use of a cyclin D/CDK4 fusion protein, in particular the hypophosphorylated form of a cyclin D/CDK4 fusion protein to determine the activation status of endogenous CDK4.

The present invention provides the proof of concept of using a cyclin D/CDK4 fusion protein reporter molecule for assessing the endogenous CDK4 activation status in a cell or cellular extract, because it is shown that said cyclin D/CDK4 fusion protein mimics the activation status of the endogenous CDK4. This invention thus provides new tools for assessing the activation status of endogenous CDK4 in a cell or cellular extract, and for screening candidate agents influencing said activation status. Such candidate agents may be suitable anti-cancer or anti-proliferative agents, agents acting on CDK4 in general, or agents acting on CDK4-related disorders in general. In addition, the assays will allow the further elucidation of the CDK4-pathway and the identification of new key components in cell cycle regulation.

In a first aspect, the present invention relates to an in vitro assay for determining the activation status of endogenous cyclin-dependent kinase 4 (CDK4) in eukaryotic cells, said assay comprising the steps of: (a) providing eukaryotic cells maintained in a quiescent state, said eukaryotic cells comprising a cyclin D/CDK4 fusion protein; (b) inducing proliferation of said eukaryotic cells; (c) isolating the cyclin D/CDK4 fusion protein from said eukaryotic cells; and (d) measuring the activation status of said isolated cyclin D/CDK4 fusion protein, thereby determining the activation status of endogenous CDK4 in said eukaryotic cells. In certain embodiments, the CDK4 part of the cyclin D/CDK4 fusion protein may be present in a hypophosphorylated form. In certain further embodiments, the CDK4 part of the cyclin D/CDK4 fusion protein may be present in a hyperphosphorylated form. Preferably, the CDK4 part of the cyclin D/CDK4 fusion protein is present in a hypophosphorylated form.

Because the activation status of the cyclin D/CDK4 fusion protein is modulated in the same way as the endogenous CDK4, the determination of the activation status of endogenous CDK4 in eukaryotic cells can be based on the determination of the activation status of the isolated cyclin D/CDK4 fusion protein. Changes in the activation state of said cyclin D/CDK4 fusion protein reflect changes in the status of the cellular components of the endogenous cyclin D/CDK4 signaling pathway and especially their ability to (de)phosphorylate CDK4.

Also disclosed herein is a method for determining the activation status of endogenous CDK4 in eukaryotic cells, said method comprising the steps of: (a) providing eukaryotic cells maintained in a quiescent state, said eukaryotic cells comprising a cyclin D/CDK4 fusion protein, wherein the CDK4 part of the fusion protein is present in a hypophosphorylated form; (b) inducing proliferation of said eukaryotic cells; (c) isolating the cyclin D/CDK4 fusion protein from said eukaryotic cells; and (d) measuring the activation status of said isolated cyclin D/CDK4 fusion protein, thereby determining the activation status of endogenous CDK4 in said eukaryotic cells.

The present assays or methods advantageously allow studying the regulation of the phosphorylation of endogenous CDK4 and the upstream mechanisms involved in the phosphorylation of endogenous CDK4. Moreover, such assays or methods allow studying the regulation of the phosphorylation of endogenous CDK4 independently of other steps of the activation process such as the expression of D-type cyclins and the assembly of D-type cyclins with CDK4.

The terms “activation status” or “activation” can be used interchangeably herein. The recitation “activation status of a cyclin-dependent kinase (CDK)” encompasses the phosphorylation status of the CDK as well as the ability of the CDK to perform its function, i.e., the ability of the CDK to phosphorylate one or more of its substrates. The activation status of endogenous CDK4 in a cell or cellular extract may vary between different levels of activation ranging between an activation status wherein most of said CDK4 proteins are unphosphorylated, i.e., hypophosphorylated state, and an activation status wherein most of said CDK4 proteins are phosphorylated, i.e., hyperphosphorylated state. The following amino acids of CDK4 are involved in the activation of CDK4: Thr172 and Pro173. Proline-directed kinases are hypothesized to be involved in the phosphorylation and activation of CDK4. Activation of CDK4 requires the binding of D-type cyclins and may require additional steps such as the binding of p21 and/or p27 proteins for stability and nuclear import of CDK4. Phosphorylation of endogenous CDK4 on Thr172 completes its activation.

The activation status of endogenous CDK4 may be determined by measuring the activation status of the cyclin D/CDK4 fusion protein, e.g. in a cell population to be tested or in a cellular extract thereof. The recitation measuring the activation status of the cyclin D/CDK4 fusion protein may encompass detecting, quantifying, and/or monitoring the activation status of the cyclin D/CDK4 fusion protein. The term “monitoring” generally refers to measuring the activation status over time. For instance, monitoring the activation status of endogenous CDK4 during cell cycle progression in a synchronized cell culture may be performed by measuring the activation status of the cyclin D/CDK4 fusion protein at one or more successive time points.

In an embodiment, an assay or method for monitoring the activation status of endogenous CDK4 in said eukaryotic cells may comprise the steps of:
providing eukaryotic cells maintained in a quiescent state, said eukaryotic cells comprising a cyclin D/CDK4 fusion protein, wherein the CDK4 part of the fusion protein is present in a hypophosphorylated form;
inducing proliferation of said eukaryotic cells;
isolating the cyclin D/CDK4 fusion protein at two or more successive time points from said eukaryotic cells; and
measuring the activation status of said isolated cyclin D/CDK4 fusion protein at said two or more successive time points, thereby monitoring the activation status of endogenous CDK4 in said eukaryotic cells.

In the present assay or method, the eukaryotic cells are maintained in a quiescent state before inducing proliferation of said eukaryotic cells. As a consequence, the eukaryotic cells to be assayed will be at least partially synchronized. Hence, the present assay or method advantageously allows studying the activation state of endogenous CDK4 during cell cycle progression of the eukaryotic cells.

The term “endogenous CDK4” generally refers to CDK4 that originates from within a eukaryotic cell, cell line, or tissue.

The term “cyclin D/CDK4 fusion protein”, as used herein, refers to a single protein comprising a cyclin D and CDK4 coupled to each other with a linker. The terms “cyclin D/CDK4 fusion protein”, or “cyclin D/CDK4 chimera” can be used interchangeably herein.

In certain embodiments, the cyclin D or D-type cyclin may be cyclin D1, cyclin D2, or cyclin D3.

The cyclin D/CDK4 fusion protein may comprise eukaryotic cyclin D. The eukaryotic cyclin D may be mammalian cyclin D, preferably human cyclin D. The cyclin D/CDK4 fusion protein may comprise eukaryotic CDK4. The eukaryotic CDK4 may be mammalian CDK4, preferably human CDK4.

Exemplary human nucleic acids, proteins, polypeptides or peptides as taught herein may be as annotated under NCBI Genbank (http://www.ncbi.nlm.nih.gov/) accession numbers given below. A skilled person will appreciate that although only one or more isoforms may be listed below, all isoforms are intended. Unless otherwise specified, the entries below are presented in the form: Name (Code; Genbank accession number for one or more representative mRNA sequences (e.g., isoforms), followed by a period and the Genbank sequence version; Genbank accession number for one or more corresponding representative amino acid sequences (e.g., isoforms), followed by a period and the Genbank sequence version):

Cyclin-dependent kinase 4, also known as CMM3 or PSK-J3 (CDK4; NM_{—}000075.3, NP_{—}000066.1)

Cyclin D1, also known as BCL1; PRAD1; U21B31; D11S287E (CCND1; NM_{—}053056.2, NP_{—}444284.1)

Cyclin D2, also known as KIAK0002 (CCND2; NM_{—}001759.3, NP_{—}001750.1)

Cyclin D3 (CCND3; NM_{—}001136126.1, NM_{—}001136017.2, NP_{—}00129598.1, NP_{—}00129489.1)

In certain preferred embodiments, cyclin D1 may comprise an amino acid sequence of SEQ ID NO. 1.

In certain preferred embodiments, CDK4 may comprise an amino acid sequence of SEQ ID NO. 2.

In an embodiment, cyclin D and CDK4 are connected by at least one linker. The at least one linker preferably comprises sufficient glycine amino acids in order to permit a correct position and interaction between cyclin D and CDK4. The linker may comprise or consist of an amino acid sequence of any one of L1, i.e., ASKGGGGSGGGGSGGGGS (SEQ ID NO. 3), L2, i.e., ASKGGGGSLEVLFQPSR (SEQ ID NO. 4), ASKGGGGSLEVLFQGPSR (SEQ ID. NO. 5), or GGGGSGGGGSGGGGS (SEQ ID NO. 6) as described previously (Huston et al., 1988, Proc. Natl. Acad. Sci. USA, 85, 5879-5883).

The cyclin D and the CDK4 moieties of the cyclin D/CDK4 fusion protein can be separated by a linker including a protease sensitive cleavage site. Proteolytic dissociation of the cyclin D/CDK4 fusion protein followed by thermic denaturation of the lysate may allow specific purification of the CDK4 moiety. The phosphorylation status of this peptide may subsequently be determined using any anti-phospho-CDK4 or any phospho-TP antibody as well as by mass spectroscopy or any other relevant technique known to those skilled in the art. Presence of the anti-phospho-CDK4 or the phospho-TP antibody on the immobilized CDK4 part of the fusion could be detected by the DELFIA technology as described herein, or by radioactive, colorimetric or chemiluminescence methods known to those skilled in the art. As such, the cyclin D/CDK4 fusion protein can perfectly be used to identify, characterize and validate any anti-phospho-CDK4 antibodies, nanobodies or aptamers.

The linker may comprise a protease cleavage consensus such as the Prescission site (GE Healthcare). Alternatively, the linker may be an intein sequence.

In a preferred embodiment, the cyclin D/CDK4 fusion protein may comprise cyclin D1 and CDK4 connected by the linker L1; said cyclin D/CDK4 fusion protein is referred to herein as D1L1K4. The D1L1K4 may comprise an amino acid sequence of SEQ ID NO. 7.

In a preferred embodiment, the cyclin D/CDK4 fusion protein may comprise cyclin D1 and CDK4 connected by the linker L2; said cyclin D/CDK4 fusion protein is referred to herein as D1L2K4. The D1L2K4 may comprise an amino acid sequence of SEQ ID NO. 8.

In a preferred embodiment, the cyclin D/CDK4 fusion protein may comprise cyclin D3 and CDK4 connected by the linker L1; said cyclin D/CDK4 fusion protein is referred to herein as D3L1K4. The D3L1K4 may comprise an amino acid sequence of SEQ ID NO. 9.

In a preferred embodiment, the cyclin D/CDK4 fusion protein may comprise cyclin D3 and CDK4 connected by the linker L2; said cyclin D/CDK4 fusion protein is referred to herein as D3L2K4. The D3L2K4 may comprise an amino acid sequence of SEQ ID NO. 10.

In a preferred embodiment, the cyclin D/CDK4 fusion protein may comprise cyclin D3 and CDK4 connected by the linker L3; said cyclin D/CDK4 fusion protein is referred to herein as D3L3K4. The D3L3K4 may comprise an amino acid sequence of SEQ ID NO. 79, encoded by the nucleotide sequence of SEQ ID NO. 78.

In a preferred embodiment, the cyclin D/CDK4 fusion protein may comprise cyclin D1 and CDK4 connected by the linker L3; said cyclin D/CDK4 fusion protein is referred to herein as D1L3K4. The D1L3K4 may comprise an amino acid sequence of SEQ ID NO. 81, encoded by the nucleotide sequence of SEQ ID NO. 80.

The reference herein to any fusion protein, protein, polypeptide, or peptide such as any cyclin D/CDK4 fusion protein may also encompass functional fragments thereof. The term “fragment” of a fusion protein, protein, polypeptide or peptide generally refers to N-terminally and/or C-terminally deleted or truncated forms of said fusion protein, protein, polypeptide or peptide, but largely retaining the functionality of the full-length reporter molecule.

The reference herein to any fusion protein, protein, polypeptide or peptide such as any cyclin D/CDK4 fusion protein may also encompass functional variants thereof, but largely retaining the functionality of the full-length reporter molecule. The term “variant” of a nucleic acid, fusion protein, protein, polypeptide or peptide refers to nucleic acid, fusion proteins, proteins, polypeptides or peptides, the sequence (i.e., nucleotide sequence or amino acid sequence, respectively) of which is substantially identical (i.e., largely but not wholly identical) to the sequence of said recited nucleic acid, fusion protein, protein or polypeptide, e.g., at least about 80% identical or at least about 85% identical, e.g., preferably at least about 90% identical, e.g., at least 91% identical, 92% identical, more preferably at least about 93% identical, e.g., at least 94% identical, even more preferably at least about 95% identical, e.g., at least 96% identical, yet more preferably at least about 97% identical, e.g., at least 98% identical, and most preferably at least 99% identical. Preferably, a variant may display such degrees of identity to a recited nucleic acid, fusion protein, protein, polypeptide or peptide when the whole sequence of the recited nucleic acid, fusion protein, protein, polypeptide or peptide is queried in the sequence alignment (i.e., overall sequence identity). Also included among fragments and variants of a nucleic acid, fusion protein, protein, polypeptide or peptide are fusion products of said nucleic acid, fusion protein, protein, polypeptide or peptide with another, usually unrelated, nucleic acid, fusion protein, protein, polypeptide or peptide.

Sequence identity may be determined using suitable algorithms for performing sequence alignments and determination of sequence identity as know per se. Exemplary but non-limiting algorithms include those based on the Basic Local Alignment Search Tool (BLAST) originally described by Altschul et al., 1990 (J. Mol. Biol., 215, 403-10), such as the “Blast 2 sequences” algorithm described by Tatusova and Madden, 1999 (FEMS Microbiol. Lett., 174, 247-250), for example using the published default settings or other suitable settings (such as, e.g., for the BLASTN algorithm: cost to open a gap=5, cost to extend a gap=2, penalty for a mismatch=−2, reward for a match=1, gap x_dropoff=50, expectation value=10.0, word size=28; or for the BLASTP algorithm: matrix=Blosum62, cost to open a gap=11, cost to extend a gap=1, expectation value=10.0, word size=3).

Where the present specification refers to or encompasses fragments and/or variants of fusion protein, proteins, polypeptides or peptides, this denotes variants and/or fragments which are “functional”, i.e., which at least partly, and preferably largely, retain the biological activity or intended functionality of the respective fusion protein, proteins, polypeptides or peptides. By means of an example and not limitation, a functional fragment and/or variant of a cyclin D/CDK4 fusion protein shall at least partly retain the biological activity of the cyclin D/CDK4 fusion protein. For example, it may retain one or more aspects of the biological activity of the cyclin D/CDK4 fusion protein, such as, e.g., the ability to phosphorylate one or more substrates, to participate in one or more cellular pathways, etc. Preferably, a functional fragment and/or variant may retain at least about 20%, e.g., at least 30%, or at least about 40%, or at least about 50%, e.g., at least 60%, more preferably at least about 70%, e.g., at least 80%, yet more preferably at least about 85%, still more preferably at least about 90%, and most preferably at least about 95% or even about 100% or higher of the intended biological activity or functionality compared to the corresponding fusion protein, protein, polypeptide or peptide. Particularly, a functional fragment or variant would retain, to at least a certain degree, the ability to allow the determination of the activation status of CDK4.

The recitation “eukaryotic cells”, as used herein, refers to cells of a eukaryotic cell line such as an immortalized cell lines, tumour cell lines, or cell lines obtained by culturing primary tumour cells.

The eukaryotic cells may be mammalian cells. The eukaryotic cells may be any cell line wherein the Rb pathway is functional. The eukaryotic cells may be mammalian tumour cell lines such as for example MCF7, T98G, or HCT116 cells, or may be immortalized normal mammalian cell lines such as for example hTERT, HME, or MCF10A.

The eukaryotic cells as used herein typically and preferably denotes human cells, but may also encompass reference to non-human animals, preferably warm-blooded animals, more preferably vertebrates, even more preferably mammals, such as, e.g., non-human primates, rodents, canines, felines, equines, ovines, porcines, and the like.

The term “hypophosphorylated form of the CDK4 part of the fusion protein”, as used herein, encompasses the phosphorylation status of the CDK4 part or protein of the cyclin D/CDK4 fusion proteins wherein none of the cyclin D/CDK4 fusion proteins or less than 50%, less than 40%, less than 30%, less than 20%, less than 10%, for example less than 5%, such as less than 4%, less than 3%, less than 2%, or less than 1% of the cyclin D/CDK4 fusion proteins are phosphorylated.

The term “hyperphosphorylated form of the CDK4 part of the fusion protein”, as used herein, encompasses the phosphorylation state of the CDK4 part or protein of the cyclin D/CDK4 fusion proteins wherein all of the cyclin D/CDK4 fusion proteins or more than 50%, more than 60%, more than 70%, more than 80%, or more than 90%, for example more than 95%, such as more than 96%, more than 97%, more than 98%, or more than 99% of the cyclin D/CDK4 fusion proteins are phosphorylated.

In certain embodiments, the assay or method comprises providing eukaryotic cells maintained in a quiescent state, said eukaryotic cells comprising a reporter molecule comprising a cyclin D/CDK4 fusion protein, wherein the CDK4 part of the fusion protein is present in a hypophosphorylated form.

The cyclin D/CDK4 fusion protein may be produced in eukaryotic cells. Preferably, the cyclin D/CDK4 fusion protein is produced in eukaryotic cells maintained in a quiescent state. Producing the cyclin D/CDK4 fusion protein in eukaryotic cells maintained in a quiescent state advantageously allows producing directly the cyclinD/CDK4 fusion protein with the CDK4 part of the fusion protein in a hypophosphorylated form.

The eukaryotic cells may be maintained in a quiescent state by culturing the eukaryotic cells in deprived growth medium. The eukaryotic cells may be maintained in a quiescent state by culturing the eukaryotic cells in the absence of serum. The eukaryotic cells may be maintained in a quiescent state by culturing the eukaryotic cells in the absence of serum and hormones such as insulin. The eukaryotic cells may be maintained in a quiescent state by culturing the eukaryotic cells in the absence of serum and in the presence of an anti-estrogen compound. The eukaryotic cells may be maintained in a quiescent state by culturing the eukaryotic cells in the absence of serum and hormones and in the presence of an anti-estrogen compound. The anti-estrogen compound preferably is fulvestrant (ICI-182,780, Faslodex, AstraZeneca), but can be equally replaced by tamoxifen, raloxifene, toremifene, or a Selective Estrogen Receptor Modulator (SERM) such as SP500263 known to those skilled in the art. For each eukaryotic cell line, it will be understood by the skilled man, which culture medium can to be used to keep the cells in a quiescent state and to induce proliferation of the eukaryotic cells.

In certain embodiments, the assay or method comprises the step (b) inducing proliferation of the eukaryotic cells.

In certain embodiments, proliferation of the eukaryotic cells may be induced by culturing the eukaryotic cells in standard growth medium. Proliferation of the eukaryotic cells may be induced by adding serum to deprived growth medium. Proliferation of the eukaryotic cells may thus be induced by culturing the eukaryotic cells in growth medium comprising serum. Proliferation of the eukaryotic cells may be induced by adding serum and hormones to deprived growth medium. Proliferation of the eukaryotic cells may thus also be induced by culturing the eukaryotic cells in growth medium comprising serum and hormones such as insulin. Other suitable compounds or compositions to induce proliferation of eukaryotic cells may be serum substitutes such as Ultrose (Pall Corporation, NY, USA) or, Panexin NTA (PAN-Biotech GmbH, Germany), or one or more growth factors selected from platelet-derived growth factor (PDGF), epidermal growth factor (EGF), insulin, or insulin-like growth factors (IGFs).

Culturing of eukaryotic cell lines is generally performed in the presence of a medium, commonly a liquid cell culture medium. Typically, the medium will comprise a basal medium formulation as known in the art. Many basal media formulations (available, e.g., from the American Type Culture Collection, ATCC; or from Invitrogen, Carlsbad, Calif.) can be used to culture the cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), BGJb, F-12 Nutrient Mixture (Ham), Iscove's Modified Dulbecco's Medium (IMDM), available from Invitrogen or Cambrex (New Jersey), and modifications and/or combinations thereof. Compositions of the above basal media are generally known in the art and it is within the skill of one in the art to modify or modulate concentrations of media and/or media supplements as necessary for the cells cultured.

Such basal media formulations contain ingredients necessary for mammalian cell development, which are known per se. By means of illustration and not limitation, these ingredients may include inorganic salts (in particular salts containing Na, K, Mg, Ca, Cl, P and possibly Cu, Fe, Se and Zn), physiological buffers (e.g., HEPES, bicarbonate), nucleotides, nucleosides and/or nucleic acid bases, ribose, deoxyribose, amino acids, vitamins, antioxidants (e.g., glutathione) and sources of carbon (e.g. glucose, sodium pyruvate, sodium acetate), etc.

For use in culture, basal media can be supplied with one or more further components. For example, additional supplements can be used to supply the cells with the necessary trace elements and substances for optimal growth and expansion. Furthermore, antioxidant supplements may be added, e.g., β-mercaptoethanol. While many basal media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin.

Plasma or serum may also be comprised in said media at a proportion (volume of plasma or serum/volume of medium) between about 0.5% and about 30%, preferably between about 1% and about 15%.

In certain embodiments, the assay or method comprises the step (c) isolating the cyclin D/CDK4 fusion protein from the eukaryotic cells. The isolation of the cyclin D/CDK4 fusion protein from the eukaryotic cells may be performed by isolating a sample of the eukaryotic cells, extracting the cyclin D/CDK4 fusion protein from the sample, and purifying the cyclin D/CDK4 fusion protein for instance using affinity purification. Preparation of a protein extract, such as a protein extract comprising the cyclin D/CDK4 fusion protein, from eukaryotic cells is well known by the skilled man in the art and illustrated in the example section.

In certain embodiments, the activation status of the isolated cyclin D/CDK4 fusion protein CDK4 may be measured by quantifying the Rb kinase activity of the isolated cyclin D/CDK4 fusion protein. In certain further embodiments, the activation status of the isolated cyclin D/CDK4 fusion protein CDK4 may be measured by detecting the phosphorylation status of the isolated cyclin D/CDK4 fusion protein, preferably by detecting phosphorylation of the isolated cyclin D/CDK4 fusion protein on T172.

The Rb kinase activity of the isolated cyclin D/CDK4 fusion protein may be assessed by incubating the isolated cyclin D/CDK4 fusion protein with retinoblastoma (Rb) protein or a fragment thereof before quantifying the phosphorylation level of the Rb protein or a fragment thereof.

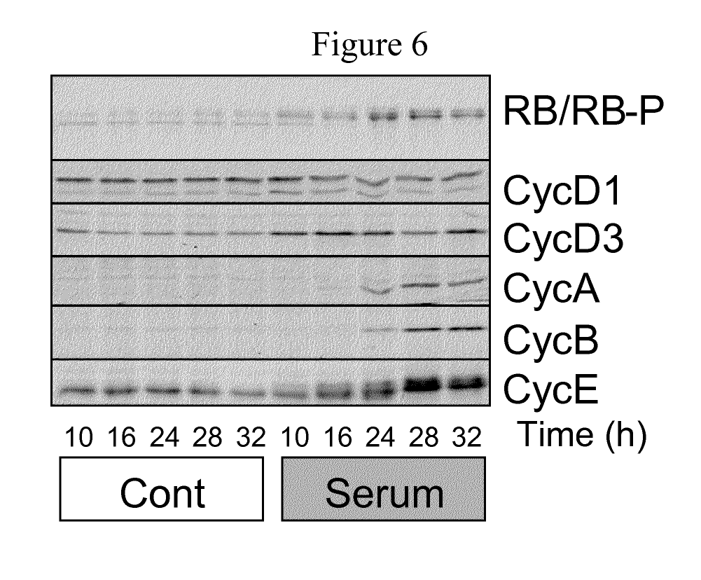

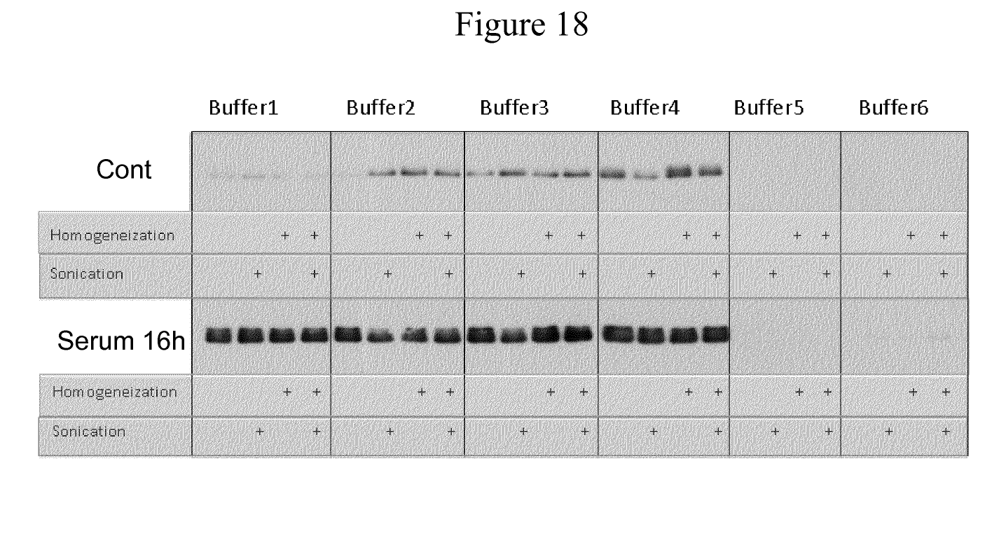

The quantification of the phosphorylation level of the Rb protein or a fragment thereof may be achieved by quantifying the amount of radioactively labelled phosphor incorporated in Rb. The quantification of the phosphorylation level of the Rb protein or a fragment thereof may be achieved by specifically detecting the phosphorylated form of Rb using a molecule specifically detecting the phosphorylated form of Rb, for example an antibody, a nanobody, or an aptamer specifically detecting the phosphorylated form of Rb. The quantification of the phosphorylation level of the Rb protein or a fragment thereof may also be achieved by mass spectroscopy. Alternatively, the phosphorylation of Rb can be evaluated by the shift in 1D gel electrophoresis migration as exemplified in FIGS. 6 and 17.

In certain embodiments, the activation status of the isolated cyclin D/CDK4 fusion protein CDK4 may be measured by detecting the phosphorylation status of the isolated cyclin D/CDK4 fusion protein.

The quantification of the phosphorylation state of the isolated cyclin D/CDK4 fusion protein may be achieved using a molecule specifically detecting phosphorylated forms of CDK4. The molecule specifically detecting phosphorylated forms of CDK4 may be an antibody, a nanobody, or an aptamer.

The molecule specifically detecting phosphorylated forms of CDK4 may interact with a phosphorylated threonine followed by a proline, or may interact with the phosphorylated T172 of CDK4.

In certain embodiments, the activation status of the isolated cyclin D/CDK4 fusion protein may be measured by detecting the phosphorylation of the cyclin D/CDK4 fusion protein on T172.

Detection of the phosphorylation of the isolated cyclin D/CDK4 fusion protein on T172 may be achieved on the immobilized native form of the isolated cyclin D/CDK4 fusion protein or may be achieved on the denaturated form of the isolated cyclin D/CDK4 fusion protein for instance after its proteolytic cleavage and immobilisation on a suitable matrix.

The quantification of phosphorylation level of the isolated cyclin D/CDK4 fusion protein on T172 may be achieved using a molecule specifically detecting the T172-phosphorylated form of the cyclin D/CDK4 fusion protein. The molecule specifically detecting the T172-phosphorylated form of the cyclin D/CDK4 fusion protein may be an antibody, a nanobody, or an aptamer.

The quantification of the phosphorylation of the isolated cyclin D/CDK4 fusion protein, in particular phosphorylation of T172 on isolated cyclin D/CDK4 fusion protein, may also be achieved by mass spectroscopy.

In certain embodiments, the activation status of the isolated cyclin D/CDK4 fusion protein may be measured using fluorescent detection technology such as Dissociation-Enhanced Lanthanide Fluorescent Immunoassay (DELFIA, Perkin-Elmer). DELFIA spectroscopy is a technique of time-resolved fluorescence (delayed transmission over the excitation). Fluorescence spectroscopy resolved in time is possible when the fluorochrome has a long disintegration time. The DELFIA technology exploits the unique properties of lanthanide fluorescence. These fluorophores have very long decay time and thus continue to emit after stopping excitation. The lanthanides also have a large difference in wavelength between the excitation light and the emission light (Stokes' shift), which makes the system very sensitive due to the virtual absence of background noise. The europium ion (Eu3+) is the most used lanthanide in the DELFIA system and has, like the other lanthanides, a single emission peak, which facilitates the possibility of multiple detections. The fluorescence of the lanthanide is measured at a time when the non-specific fluorescence is almost zero i.e., 400-800 μs after cessation of excitation.

In certain embodiments, the activation status of the isolated cyclin D/CDK4 fusion protein may be measured by the DELFIA technology wherein two different antibodies with two lanthanides are used. A first lanthanide may be coupled to an antibody directed against the phosphorylated CDK4 protein of the isolated cyclin D/CDK4 fusion protein and a second lanthanide may be coupled to an antibody against the total cyclin D/CDK4 fusion protein for instance against the CDK4 part of the fusion protein. The antibodies may be added to the reporter molecule and an activation solution may be added. The low pH of the activation solution decouples the lanthanide antibody in a few minutes. The free lanthanide will form a chelate with the components of the activating solution in a protective micelle. The chelates can be excited and the fluorescence emission of two filters measured: a filter or channel passing the wavelength of the emission peak of the first lanthanide and a filter or channel passing the wavelength of the emission peak of the second lanthanide. In this way, the fluorescence ratio between the two channels can be measured and hence the ratio of the phosphorylated cyclin D/CDK4 fusion protein versus total cyclin D/CDK4 fusion protein may be determined.

In certain embodiments, an assay or method for evaluating the effect of a candidate agent on the activation status of endogenous CDK4, may comprise, prior to or upon inducing proliferation of said eukaryotic cells, the step of incubating or contacting said eukaryotic cells with at least one candidate agent.

This step advantageously allows to identify activating kinases of endogenous CDK4 such as proline-directed kinases or to identify inhibitory molecules acting upstream of endogenous cyclinD/CDK4 complexes or directly inhibiting the activation of endogenous cyclin D/CDK4 complexes.

In certain embodiments, the assay or method for evaluating the effect of a candidate agent on the activation status of endogenous CDK4 in eukaryotic cells may comprise the steps of:
providing eukaryotic cells, said eukaryotic cells comprising a cyclin D/CDK4 fusion protein;
incubating said eukaryotic cells with at least one candidate agent,
isolating the cyclin D/CDK4 fusion protein from said eukaryotic cells;
measuring the activation status of said isolated cyclin D/CDK4 fusion protein; and
evaluating the effect of the candidate agent on the activation status of endogenous CDK4 in said eukaryotic cells by comparing the activation status of said isolated cyclin D/CDK4 fusion protein between said eukaryotic cells incubated with the candidate agent and eukaryotic cells left untreated.

In certain embodiments, the assay or method for evaluating the effect of a candidate agent on the activation status of endogenous CDK4 in eukaryotic cells may comprise the steps of:
providing eukaryotic cells maintained in a quiescent state, said eukaryotic cells comprising a cyclin D/CDK4 fusion protein, wherein the CDK4 part of the fusion protein is present in a hypophosphorylated form;
incubating said eukaryotic cells with at least one candidate agent,
inducing proliferation of said eukaryotic cells;
isolating the cyclin D/CDK4 fusion protein from said eukaryotic cells;
measuring the activation status of said isolated cyclin D/CDK4 fusion protein; and
evaluating the effect of the candidate agent on the activation status of endogenous CDK4 in said eukaryotic cells by comparing the activation status of said isolated cyclin D/CDK4 fusion protein between said eukaryotic cells incubated with the candidate agent and said eukaryotic cells left untreated.

It will be understood by the skilled person that the assays or methods as taught herein may comprise measuring the activation status of the cyclin D/CDK4 fusion protein in comparison with a control. The control may be said eukaryotic cells left untreated.

Importantly, such assays or methods may allow identifying candidate agents activating or inhibiting endogenous cyclin D/CDK4 complexes. Said agents influencing CDK4 signaling or activation can result in candidate therapeutic agents for treating CDK4-related diseases. The term “CDK4-related diseases” may encompass any disease or disorder wherein the activation of CDK4 is deregulated such as certain types of cancer, e.g. gliomas and sarcomas, and proliferative diseases or disorders, e.g. restenosis or psoriasis.

In addition, the assay can be used to screen molecules or therapeutics for their effect on cell-proliferation. That is, when a hypophosphorylated fusion construct is used, increase in phosphorylation in presence of the candidate agents points towards stimulation of proliferation. The assay furthermore allows screening molecules or therapeutics able to inhibit CDK4 activating kinases. In CDK4-related diseases, CDK4 activating kinases may be constitutively active. The present assays or methods as taught herein may advantageously allow screening candidate agent able to inhibit or inactivate these CDK4 activating kinases.

The assays embodying the principles of the present invention advantageously allow high throughput screening for activating kinases of endogenous CDK4 in an efficient and inexpensive way. The present assays hence allow studying the upstream signalling cascade of endogenous CDK4. For instance, the assays of the present invention allow high throughput screening of siRNA or mRNA libraries of the kinome to identify kinases involved in the inhibition or activation of endogenous CDK4. Additionally, the present assays also allow high throughput screening of compounds able to inactivate the endogenous cyclin D/CDK4 complex or one or more of the upstream kinases. The assays also allow determining the impact of the overexpression of any kinase or signalling molecule on the activation of endogenous CDK4, by e.g. screening a cDNA or mRNA library of candidate proteins. The Broad Institute and MIT Human Kinase ORF collection from Addgene consisting of 559 distinct human kinases and kinase-related protein open reading frames (ORFs) in pDONR-223 Gateway® Entry vectors can be an important instrument in this respect.

The at least one candidate agent may be selected from the group consisting of a biological sample, a protein, a nucleic acid, an siRNA, a microRNA, a chemical compound, and a small molecule. The biological sample may be a cell extract such as a eukaryotic cell extract or a prokaryotic cell extract, tumor biopsy, tumor exudates, blood etc.

Suitable non-limiting examples of chemical compounds include roscovitine and its derivatives such as CR8, PD033299, BS-181, or combinations thereof. Libraries of specific and non-specific kinase inhibitors are commercially available (Selleckbio, SelleckChem, enzolifesciences, Cayman chemicals, Millipore, Tocris, aachembio, vichem synmedchem, . . . )

Suitable non-limiting examples of siRNA or microRNA include RNA molecules which are directed against candidate activating kinases of CDK4. Possible candidate activating kinases of CDK4 include proline-directed kinases (PDK); mitogen-activated protein (MAP) kinases such as c-Jun N-terminal kinases (JNKs) and cyclin-dependent kinases (CDKs) such as CDK1 to CDK20; glycogen synthase kinases (GSKs) such as GSK-3, Homeodomain-interacting protein kinases (HIPKs); and kinases related to both MAPKs and CDKs.

A suitable siRNA library, although partial is the CMGC Kinase G-004500 of Dharmacon. The G-03505_Human_siGENOME_Protein_Kinase of Dharmacon is a suitable alternative as it cover all known kinases of the human genome. Alternative libraries are also provided by Qiagen (FlexiPlate siRNA Gene Family), Invitrogen (Silencer Select Human Kinase siRNA Library V4), Sigma (MISSION® siRNA Human Kinase Panel), Ambion (Silencer™ Kinase siRNA Library) Abnova (Kinase siRNA library SRL003), Bioneer (AccuTarget Human Kinase siRNA Set),

In an aspect, the present invention relates to an in vitro assay for determining the activation status of endogenous cyclin-dependent kinase 4 (CDK4) in a eukaryotic cell extract, said assay comprising the steps of: (a) providing a cyclin D/CDK4 fusion protein; (b) contacting the cyclin D/CDK4 fusion protein with a eukaryotic cell extract; and (c) measuring the activation status of said cyclin D/CDK4 fusion protein, thereby determining the activation status of endogenous CDK4 in said eukaryotic cell extract. In certain embodiments, the CDK4 part of the cyclin D/CDK4 fusion protein may be present in a hypophosphorylated form. In certain further embodiments, the CDK4 part of the cyclin D/CDK4 fusion protein may be present in a hyperphosphorylated form. Preferably, the CDK4 part of the cyclin D/CDK4 fusion protein is present in a hypophosphorylated form.

The above recitation “determining the activation status of endogenous CDK4 in said eukaryotic cell extract” may encompass determining the capability or potential of the cell extract to activate the cyclin D/CDK4 fusion protein.

Also disclosed herein is a method for determining the activation status of eukaryotic CDK4, said method comprising the steps of: (a) providing a cyclin D/CDK4 fusion protein, wherein the CDK4 part of the fusion protein is present in a hypophosphorylated form; (b) contacting the cyclin D/CDK4 fusion protein with a eukaryotic cell extract; and (c) measuring the activation status of said cyclin D/CDK4 fusion protein, thereby determining the activation status of endogenous CDK4 in said eukaryotic cell extract.

The present assays or methods advantageously allow to study the regulation of the phosphorylation of endogenous CDK4 in a eukaryotic cell extract, for instance in a eukaryotic cell extract prepared from a tumor. Advantageously, the present assays or methods allow studying the regulation of the phosphorylation of endogenous CDK4 in a eukaryotic cell extract independently of the expression of D-type cyclins and the assembly of cyclin D with CDK4.

In certain embodiments, the present assay or method comprises the step of (a) providing a cyclin D/CDK4 fusion protein. The CDK4 part of the fusion protein may be present in a hyperphosphorylated form. Preferably, the CDK4 part of the fusion protein is present in a hypophosphorylated form.

In certain embodiments, a cyclin D/CDK4 fusion protein may be provided by producing the cyclin D/CDK4 fusion protein, extracting the cyclin D/CDK4 fusion and purifying the cyclin D/CDK4 fusion.

In certain embodiments, the cyclin D/CDK4 fusion protein provided in the assays or methods as taught herein may be produced in prokaryotic cells. In certain further embodiments, the cyclin D/CDK4 fusion protein provided in the assays or methods as taught herein may be produced in eukaryotic cells and subsequently dephosphorylated by a phosphatase such as the lambda phosphatase. This advantageously allows obtaining eukaryotic cells wherein the CDK4 part of the fusion protein is present in a hypophosphorylated form. The cyclin D/CDK4 fusion protein provided in the assays or methods as taught herein may also be produced in eukaryotic cells maintained in a quiescent state. Producing the cyclin D/CDK4 fusion protein in eukaryotic cells maintained in a quiescent state advantageously allows producing directly the cyclinD/CDK4 fusion protein with the CDK4 part of the fusion protein in a hypophosphorylated form.

The cyclin D/CDK4 fusion protein may be extracted from the cells such as prokaryotic cells or eukaryotic cells by any method known in the art. The cyclin D/CDK4 fusion protein may be purified by affinity purification or using the Avi tag or any other tag coupled to the cyclin D/CDK4 fusion protein. Methods for affinity purification are known in the art and are illustrated in the example section.

In certain embodiments, the present assay or method comprises the step of (b) contacting the cyclin D/CDK4 fusion protein with a eukaryotic cell extract.

The eukaryotic cell extract may be obtained from a eukaryotic cell line such as an established cell line or a tumor cell line. The eukaryotic cell extract may also be obtained from tissue such as a tumor biopsy, an exudate, etc.

The eukaryotic cell extract may be obtained from untreated eukaryotic cells. The eukaryotic cell extract may also be obtained from synchronized eukaryotic cells. Furthermore, the eukaryotic cell extract may be obtained from eukaryotic cells incubated with at least one candidate agent.

In certain embodiments, an assay or method as taught herein may comprise obtaining the eukaryotic cell extract from untreated eukaryotic cells. Such cell extract obtained from untreated cells may advantageously allow determining the activation status of endogenous CDK4 in tumour cell lines, tumours, tumour-associated fluids or tissues, pleural effusions, exudates etc.

In certain further embodiments, an assay or method as taught herein may comprise obtaining the eukaryotic cell extract from synchronized eukaryotic cells. Such cell extract obtained from synchronized eukaryotic cells may allow determining the activation status of endogenous CDK4 during cell cycle progression. Eukaryotic cells may be synchronized by any technique known in the art for cell cycle synchronization such as nutrient deprivation or nutritional blockage, the addition of chemical compounds for instance arresting the cells at the G1-S or G2-M cell cycle transitions, or cell sorting.

In certain embodiments, an assay or method for evaluating the effect of a candidate agent on the activation status of endogenous CDK4 may comprise obtaining the eukaryotic cell extract from eukaryotic cells incubated with at least one candidate agent.

In certain embodiments, an assay or method for evaluating the effect of a candidate agent on the activation status of endogenous CDK4 in a eukaryotic cell extract may comprise the steps of:
incubating eukaryotic cells with a candidate agent,
preparing a eukaryotic cell extract from said eukaryotic cells;
providing a cyclin D/CDK4 fusion protein, wherein the CDK4 part of the fusion protein is present in a hypophosphorylated form;
contacting the cyclin D/CDK4 fusion protein with the eukaryotic cell extract;
measuring the activation status of the cyclin D/CDK4 fusion protein; and
evaluating the effect of the candidate agent on the activation status of endogenous CDK4 in said eukaryotic cell extract by comparing the activation status of said isolated cyclin D/CDK4 fusion protein between said eukaryotic cell extract prepared from said eukaryotic cells incubated with the candidate agent and a eukaryotic cell extract prepared from eukaryotic cells left untreated.

In certain further embodiments, an assay or method for evaluating the effect of a candidate agent on the activation status of endogenous CDK4 may comprise obtaining a eukaryotic cell extract and incubating the eukaryotic cell extract with at least one candidate agent.

In certain embodiments, an assay or method for evaluating the effect of a candidate agent on the activation status of endogenous CDK4 in a eukaryotic cell extract may comprise the steps of:
preparing a eukaryotic cell extract from eukaryotic cells;
providing a cyclin D/CDK4 fusion protein, wherein the CDK4 part of the fusion protein is present in a hypophosphorylated form;
incubating the eukaryotic cell extract with a candidate agent, thereby obtaining a treated eukaryotic cell extract;
contacting the cyclin D/CDK4 fusion protein with the treated eukaryotic cell extract;
measuring the activation status of the cyclin D/CDK4 fusion protein; and
evaluating the effect of the candidate agent on the activation status of endogenous CDK4 by comparing the activation status of said isolated cyclin D/CDK4 fusion protein between said treated eukaryotic cell extract, i.e., cell extract incubated with the candidate agent, and a eukaryotic cell extract left untreated.

It will be understood by the skilled person that the assays or methods as taught herein may comprise measuring the activation status of the cyclin D/CDK4 fusion protein in comparison with a control. The control may be a cell extract of said eukaryotic cells left untreated or may be said eukaryotic cell extract left untreated.

Advantageously, such assays allow to identify activating kinases of endogenous CDK4 such as proline-directed kinases, or to identify inhibitory molecules acting upstream of cyclin D/CDK4 complexes or directly inhibiting the activation of cyclin D/CDK4 complexes.

The candidate agent may for instance be an siRNA. Hence, in certain embodiments, an assay or method for identifying an activating kinase of endogenous CDK4 in eukaryotic cells may comprise the steps of:
providing eukaryotic cells maintained in a quiescent state, said eukaryotic cells comprising a cyclin D/CDK4 fusion protein, wherein the CDK4 part of the fusion protein is present in a hypophosphorylated form;
incubating said eukaryotic cells with an siRNA directed against a candidate activating kinase,
inducing proliferation of said eukaryotic cells;
isolating the cyclin D/CDK4 fusion protein from said eukaryotic cells;
measuring the activation status of said isolated cyclin D/CDK4 fusion protein; and
identifying the candidate activating kinase as an activating kinase of endogenous CDK4 in said eukaryotic cells when the isolated cyclin D/CDK4 fusion protein is not activated, or by comparing the activation status of said isolated cyclin D/CDK4 fusion protein between said eukaryotic cells incubated with said siRNA and eukaryotic cells left untreated.

In certain embodiments, an assay or method for identifying an activating kinase of endogenous CDK4 in a eukaryotic cell extract may comprise the steps of:
incubating eukaryotic cells with an siRNA directed against a candidate activating kinase,
preparing a eukaryotic cell extract from said eukaryotic cells;
providing a cyclin D/CDK4 fusion protein, wherein the CDK4 part of the fusion protein is present in a hypophosphorylated form;
contacting the cyclin D/CDK4 fusion protein with the eukaryotic cell extract;
measuring the activation status of the cyclin D/CDK4 fusion protein; and
identifying the candidate activating kinase as an activating kinase of endogenous CDK4 in a eukaryotic cell extract when the cyclin D/CDK4 fusion protein is not activated, or by comparing the activation status of said isolated cyclin D/CDK4 fusion protein between said eukaryotic cell extract obtained from said eukaryotic cells incubated with said siRNA and a eukaryotic cell extract obtained from said eukaryotic cells left untreated.

Such assays or methods may advantageously allow identifying activating kinases of endogenous CDK4 such as proline-directed kinases, or identifying kinases involved in the upstream pathway of the activation of endogenous cyclin/CDK4 complexes.

The candidate agent may for instance be a chemical compound. Thus, in certain embodiments, the assay or method for identifying a chemical compound affecting the activation status of endogenous CDK4 in eukaryotic cells may comprise the steps of:
providing eukaryotic cells maintained in a quiescent state, said eukaryotic cells comprising a cyclin D/CDK4 fusion protein, wherein the CDK4 part of the fusion protein is present in a hypophosphorylated form;
incubating said eukaryotic cells with a chemical compound,
inducing proliferation of said eukaryotic cells;
isolating the cyclin D/CDK4 fusion protein from said eukaryotic cells;
measuring the activation status of said isolated cyclin D/CDK4 fusion protein; and
identifying whether the chemical compound affects the activation status of endogenous CDK4 in eukaryotic cells by comparing the activation status of said isolated cyclin D/CDK4 fusion protein between said eukaryotic cells incubated with said chemical compound and eukaryotic cells left untreated.

In certain embodiments, an assay or method for identifying a chemical compound affecting the activation status of endogenous CDK4 in a eukaryotic cell extract may comprise the steps of:
incubating eukaryotic cells with a chemical compound,
preparing a eukaryotic cell extract from said eukaryotic cells;
providing a cyclin D/CDK4 fusion protein, wherein the CDK4 part of the fusion protein is present in a hypophosphorylated form;
contacting the cyclin D/CDK4 fusion protein with the eukaryotic cell extract;
measuring the activation status of the cyclin D/CDK4 fusion protein; and
identifying whether the chemical compound affects the activation status of endogenous CDK4 in said eukaryotic cell extract by comparing the activation status of said isolated cyclin D/CDK4 fusion protein between said eukaryotic cell extract prepared form eukaryotic cells incubated with the chemical compound and a eukaryotic cell extract prepared from eukaryotic cells left untreated.

In certain further embodiments, an assay or method for identifying a chemical compound, such as a small molecule, affecting the activation status of endogenous CDK4 in a eukaryotic cell extract may comprise the steps of:
preparing a eukaryotic cell extract from eukaryotic cells;
providing a cyclin D/CDK4 fusion protein, wherein the CDK4 part of the fusion protein is present in a hypophosphorylated form;
contacting or incubating the eukaryotic cell extract with a chemical compound, thereby obtaining a treated eukaryotic cell extract;
contacting the cyclin D/CDK4 fusion protein with the treated eukaryotic cell extract;
measuring the activation status of the cyclin D/CDK4 fusion protein; and
identifying whether the chemical compound affects the activation status of endogenous CDK4 in said eukaryotic cell extract by comparing the activation status of said isolated cyclin D/CDK4 fusion protein between said eukaryotic cell extract incubated with the chemical compound and a eukaryotic cell extract left untreated.

Importantly, such assays or methods may allow identifying chemical compounds activating or inhibiting endogenous cyclin D/CDK4 complexes or its activation by another kinase.

The candidate agent may for instance be a candidate activating kinase of CDK4. The eukaryotic cells may be stably or transiently transfected with a nucleic acid encoding a candidate activating kinase of CDK4. Preferably, the expression of the nucleic acid encoding a candidate activating kinase of CDK4 is controlled by an inducible promoter.

Hence, in certain embodiments, an assay or method for identifying an activating kinase of endogenous CDK4 in eukaryotic cells may comprise the steps of:
providing eukaryotic cells maintained in a quiescent state, said eukaryotic cells comprising a cyclin D/CDK4 fusion protein, wherein the CDK4 part of the fusion protein is present in a hypophosphorylated form, and said eukaryotic cells comprising a nucleic acid encoding a candidate activating kinase;
inducing the expression of said candidate activating kinase,
inducing proliferation of said eukaryotic cells;
isolating the cyclin D/CDK4 fusion protein from said eukaryotic cells;
measuring the activation status of said isolated cyclin D/CDK4 fusion protein; and
identifying the candidate activating kinase as an activating kinase of endogenous CDK4 in eukaryotic cells when the isolated cyclin D/CDK4 fusion protein is activated or by comparing the activation status of said isolated cyclin D/CDK4 fusion protein between said eukaryotic cells wherein the expression of the candidate activating kinase is induced and non-induced eukaryotic cells.

In certain embodiments, an assay or method for identifying an activating kinase of endogenous CDK4 in a eukaryotic cell extract may comprise the steps of:
providing eukaryotic cells, said eukaryotic cells comprising a nucleic acid encoding a candidate activating kinase;
inducing the expression of said candidate activating kinase,
preparing a eukaryotic cell extract from said eukaryotic cells;
providing a cyclin D/CDK4 fusion protein, wherein the CDK4 part of the fusion protein is present in a hypophosphorylated form;
contacting the cyclin D/CDK4 fusion protein with the eukaryotic cell extract;
measuring the activation status of the cyclin D/CDK4 fusion protein; and
identifying the candidate activating kinase as an activating kinase of endogenous CDK4 in the eukaryotic cell extract when the isolated cyclin D/CDK4 fusion protein is activated or by comparing the activation status of said isolated cyclin D/CDK4 fusion protein between said eukaryotic cell extract obtained from eukaryotic cells wherein the expression of the candidate activating kinase is induced and a eukaryotic cell extract obtained from non-induced eukaryotic cells.

In certain embodiments, said eukaryotic cells may comprise an expression vector, preferably an inducible expression vector, comprising a nucleic acid sequence encoding a candidate activating kinase and the expression of the candidate activating kinase may be induced in said eukaryotic cells.

In certain embodiments, an assay or method for identifying an activating kinase of endogenous CDK4 in eukaryotic cells may comprise the steps of:
providing eukaryotic cells maintained in a quiescent state, said eukaryotic cells comprising a cyclin D/CDK4 fusion protein, wherein the CDK4 part of the fusion protein is present in a hypophosphorylated form;
inducing in said eukaryotic cells the expression of a candidate activating kinase with an expression vector, preferably an inducible expression vector;
inducing proliferation of said eukaryotic cells;
isolating the cyclin D/CDK4 fusion protein from said eukaryotic cells;
measuring the activation status of said isolated cyclin D/CDK4 fusion protein; and
identifying the candidate activating kinase as an activating kinase of endogenous CDK4 when the isolated cyclin D/CDK4 fusion protein is activated or by comparing the activation status of said isolated cyclin D/CDK4 fusion protein between said eukaryotic cells wherein the expression of the candidate activating kinase is induced and non-induced eukaryotic cells.

In certain embodiments, an assay or method for identifying an activating kinase of endogenous CDK4 in a eukaryotic cell extract may comprise the steps of:
inducing in a eukaryotic cells the expression of a candidate activating kinase with an expression vector, preferably an inducible expression vector;
preparing a eukaryotic cell extract from said eukaryotic cells;
providing a cyclin D/CDK4 fusion protein, wherein the CDK4 part of the fusion protein is present in a hypophosphorylated form;
contacting the cyclin D/CDK4 fusion protein with the eukaryotic cell extract;
measuring the activation status of the cyclin D/CDK4 fusion protein; and
identifying the candidate activating kinase as an activating kinase of endogenous CDK4 when the cyclin D/CDK4 fusion protein is activated or by comparing the activation status of said isolated cyclin D/CDK4 fusion protein between said eukaryotic cell extract obtained from eukaryotic cells wherein the expression of the candidate activating kinase is induced and a eukaryotic cell extract obtained from non-induced eukaryotic cells.

In certain embodiments, the expression vector is an inducible vector.

Such assays or methods may advantageously allow identifying activating kinases of endogenous CDK4 such as proline-directed kinases, or identifying kinases involved in the upstream pathway of the activation of endogenous cyclin/CDK4 complexes.

The candidate agent may also be any industrial product or material, for which it is important to know its effect on CDK4 signalling, especially proliferation-related signaling.

The assays or methods as taught herein may advantageously allow evaluating or testing the effect of the industrial product or material on the CDK4 activation status in eukaryotic cells or cell lines. The assays or methods as taught herein may allow determining whether an industrial product or material influences the cell cycle progression of eukaryotic cells or cell lines. The assays or methods as taught herein may hence allow determining the toxicity of the industrial product or material in eukaryotic cells or cell lines.

In a further aspect, the present invention relates to a reporter molecule configured for determining the activation status of endogenous CDK4, wherein said reporter molecule comprises a cyclin D/cyclin-dependent-kinase 4 (CDK4) fusion protein and an Avi tag. Preferably, the reporter molecule comprises from its amino terminus to its carboxyl terminus: a cyclin D/cyclin-dependent-kinase 4 (CDK4) fusion protein and an Avi tag. More preferably, the CDK4 protein of the cyclin D/CDK4 fusion protein is directly coupled to the Avi tag.

In certain embodiments, the Avi tag is part of the dual SB1-Avi tag for tandem purification.

Cyclin D may be selected from cyclin D1, cyclin D2, or cyclin D3.

In certain embodiments, said reporter molecule comprises an amino acid sequence of any one of SEQ ID No. 7, SEQ ID No. 8, SEQ ID No. 9, SEQ ID No. 10, SEQ ID NO. 79, or SEQ ID NO. 81.

The reporter molecule as taught herein may further comprise one or more purification tags selected from Avi tag, poly-histidine tag, glutathion-S-transferase (GST), maltose binding protein (MBP), SNAP/CLIP tag, Halo tag, Strep tag, and the synaptobrevin SB1, Flag, V5, Myc, HA epitopes. These tags may advantageously allow additional purification steps of the reporter molecule such as affinity purification.

The reporter molecule as taught herein may comprise a cleavable sequence. In certain embodiments, the one or more purification tags may be separated from the cyclin D/CDK4 fusion protein by a cleavable sequence. In certain embodiments, the cyclin D/CDK4 fusion protein or the one or more purification tags may comprise a cleavable sequence such as a sequence cleavable by a protease. The cleavable sequence may be a tobacco etch virus (TEV) sequence such as TEV1 or TEV2. The cleavable sequence may also be an intein sequence.

The reporter molecule as taught herein may comprise a reporter protein such as an enhanced green fluorescent protein (EGFP) or Luciferase.

In certain embodiments, said reporter molecule may comprise an amino acid sequence of any one of SEQ ID No. 11, SEQ ID NO. 12, SEQ ID NO. 13, and SEQ ID No. 14.

In an aspect, the present invention also provides a reporter system comprising the reporter molecule as taught herein and the biotin ligase BirA.

The term “biotin ligase BirA”, as used herein, refers to the E. coli bifunctional biotin-[acetylCoA carboxylase] holoenzyme synthetase/DNA-binding transcriptional repressor, bio-5′-AMP-binding, also known as BirA, bioR; dhbB; ECK3965; JW3941 (bir A; NC_{—}000913.2; NP_{—}418404.1), or a functional fragment or variant thereof such as a humanized version of the E. coli BirA protein.

In certain embodiments, said reporter system may comprise a biotin ligase BirA encoded by a nucleic acid sequence of any one of SEQ ID No. 15 and SEQ ID No. 17.

In certain embodiments, said reporter system may comprise an amino acid sequence of any one of SEQ ID No. 16 and SEQ ID No. 18.

Different ways to drive transiently or in a stable way the expression of the cyclin D/CDK4 fusion protein, the BirA and optionally other components of the reporter system such as transcriptional regulators, selection markers, or detection markers are possible as appreciated by those skilled in the art.

Expression vectors can be transiently introduced in recipient cells by common electroporation methods or transfection methods such as the calcium phosphate method, the lipofection method, or the use of polyethylenimine (PEI) derivatives. Linearized expression vectors can be used to generate stable cell lines provided that they bear appropriate selection markers. Transient expression of transgenes can also be insured by the use of modified baculovirus vectors in which insect promoter sequences driving the expression of the transgenes are replaced by promoter sequence active in mammalian cells. Adenoviruses or adenovirus-associated viruses are an alternative way to transiently express the desirable transgenes in mammalian cells. In a preferred embodiment of the above described assay, cell lines stably expressing the desired transgenes are generated using lentiviral vectors or any other suitable retroviruses.

In one particular configuration of the expression system, all components required for the assay may be present in a single viral vector. This vector contains an expression module for the Avi-tagged cyclin D/CDK4 fusion protein under the control of a tet response element. This vector also contains a bicistronic expression module under the control of a constitutive promoter. This bicistronic module drives the expression of the rTTA3 regulator separated by an IRES sequence from the BirA biotine ligase fused to a selection marker. The rTTA3 regulator will induce the expression of the cyclin D/CDK4 fusion protein following the addition of the doxycycline molecule to the cells. The BirA biotine ligase may biotinylate the Avi-tag of the cyclin D/CDK4 fusion protein. The mCherry sequence fused to the BirA biotine ligase codes for a fluorescent protein usable to determine the proportion of cells expressing the rTTA3 and the BirA/mCherry fusion, and to enrich this population by Fluorescence-activated cell sorting (FACS). Alternatively, the BirA biotine ligase may be fused in frame to any antibiotic resistance gene in a way that does not interfere with its ability to biotinylate the Avi-tag. Selection of the population expressing the transgene occur by incubating the cells with the appropriate antibiotic rendered inactive by the above mentioned resistance gene. Such resistance gene can confer resistance to neomycin, blasticidin, puromycin, zeocin, hygromycin in a non-limitative way. In a particular mode of the single vector configuration, the cyclin D/CDK4 fusion protein is part of a bicistronic expression module under the control of a constitutive promoter. In the bicistronic expression module, the cyclin D/CDK4 fusion protein is separated by an IRES sequence from the BirA biotine ligase fused to a selection marker as described herein.

In a second particular configuration of the expression system, all components required for the assay may be present on two distinct viral vectors. The rTTA3 regulator may be expressed under the control of a constitutive promoter. It may be part of a bicistronic expression module in which the rTTA3 regulator is separated by an IRES sequence from a selection marker. This marker can be a fluorescent protein, or a protein conferring to the cells the resistance to an antibiotic. The fluorescent protein will be used to determine the proportion of cells expressing the rTTA3 and to enrich this population by FACS sorting. The rTTA3 regulator may acts in trans to drive, in the presence of doxycyclin, the expression of an expression module introduced in the cell by a second vector. This module includes the Avi-tagged cyclin D/CDK4 fusion protein and the BirA/mCherry fusion separated from the former by an IRES sequence. The BirA biotine ligase may biotinylate in cis the Avi-tag of the cyclin D/CDK4 fusion protein. The mCherry sequence fused to the BirA biotine ligase codes for a fluorescent protein usable to determine the proportion of cells expressing the rTTA3 and the BirA/mCherry fusion and to enrich this population by FACS sorting. Alternatively, the BirA biotine ligase can be fused in frame to any antibiotic resistance gene in a way that does not interfere with its ability to biotinylate the Avi-tag. Selection of the population expressing the transgene occur by incubating the cells with the appropriate antibiotic rendered inactive by the above mentioned resistance gene. Such resistance gene can confer resistance to neomycin, blasticidin, puromycin, zeocin, hygromycin in a non-limitative way. Alternatively, the expression of BirA/mCherry fusion present on the second vector can be controlled by a constitutive promoter. In this case, the BirA biotine ligase can be fused in frame to any antibiotic resistance gene in a way that does not interfere with its ability to biotinylate the Avi-tag. Selection of the population expressing the transgene can occur by incubating the cells with the appropriate antibiotic rendered inactive by the above mentioned resistance gene.

In a further alternative, the rTTA3 present on the first vector can be replaced by the BirA biotine ligase or its fusion with a fluorescent molecule or an antibiotic resistance gene. If the rTTA3 present on the first vector is be replaced only by the BirA biotine ligase, the vector should also include either a fluorescent molecule or an antibiotic resistance gene under the control of an IRES sequence or a constitutive promoter. In any of these cases, the rTTA3 regulator can be expressed under the control of a constitutive promoter located on the second vector. In this vector, the rTTA3 regulator sequence will be followed by an IRES sequence driving the expression of a fluorescent molecule or an antibiotic resistance gene. The second vector will also include an expression module for the Avi-tagged cyclin D/CDK4 fusion under the control of a tet response element. Expression of the Avi-tagged cyclin D/CDK4 fusion can be regulated in cis by the rTTA3 regulator according to the presence of doxycyclin. The expressed Avi-tagged cyclin D/CDK4 fusion can in this case be constitutively biotinylated in trans by the BirA biotine ligase expressed from the first vector. In a last alternative, the expression of the Avi-tagged cyclin D/CDK4 fusion protein can be driven by a constitutive promoter on the second vector. This second vector will also drive the expression of a fluorescent molecule or an antibiotic resistance gene either through the use of an IRES sequence following the Avi-tagged cyclin D/CDK4 fusion sequence or through the use of a second independent constitutive promoter. In this last setting, the Avi-tagged cyclin D/CDK4 fusion can be constitutively expressed and biotinylated in trans by the BirA biotine ligase expressed from the first vector.

In a third particular configuration of the expression system, all components required for the assay may be present on three distinct viral vectors. In this case, both the regulation of the expression of the Avi-tagged cyclin D/CDK4 fusion by the rTTA3 regulator according to the presence of doxycyclin and its biotinylation by BirA will occur in trans. For example, three different fluorescent molecules or antibiotic resistance genes may be expressed together with the respective transgenes via an IRES sequence. Alternatively, the expression of fluorescent molecules or antibiotic resistance genes may be driven by independent constitutive promoters. If the BirA biotine ligase sequence of the vector is replaced by BirA/mCherry fusion or a BirA biotine ligase fused in frame to any antibiotic resistance gene in a way that does not interfere with its ability to biotinylate the Avi-tag, no further use of fluorescent or selection marker is required.

The one skilled in the art would appreciate that alternative inducible expression system can advantageously replace the rTTA3 regulator used in the example above. Non limitative examples are: the Lacl repressor of the Lac Switch system (Stratagene), the modified ecdysome receptor of the GAL4-DBD/hPR-LBD/p65-AD regulatory fusion protein of the GeneSwitch™ System (Life technologies), the pFB-ERV vectors (stratagene), the SparQ™ Cumate Switch system from system biosciences, etc.

In the case that the expression of the desired transgenes is driven by more than one vector, infection and/or selection of the appropriate cell population can occur sequentially or in parallel depending on the fluorescent or selection markers used as appreciated by those skilled in the art.

In a further aspect, the present invention provides the use of a reporter molecule for determining the activation status of endogenous CDK4, wherein said reporter molecule comprises a cyclin D/cyclin-dependent-kinase 4 (CDK4) fusion protein. The reporter molecule may further comprise an affinity tag.

In an aspect, the present invention relates to a kit for determining the activation status of endogenous CDK4 in eukaryotic cells or a eukaryotic cell extract, said kit comprising a eukaryotic cell line and a cyclin D/CDK4 fusion protein. In certain embodiments, the CDK4 part of the fusion protein may be in the hyperphosphorylated form. In certain preferred embodiments, the CDK4 part of the fusion protein may be in the hypophosphorylated form.

In a further aspect, the present invention relates to a kit for determining the activation status of endogenous CDK4 in eukaryotic cells or a eukaryotic cell extract, said kit comprising a eukaryotic cell line and a nucleic acid encoding a cyclin D/CDK4 fusion protein. In certain embodiments, the genomic material of said eukaryotic cell line may comprise said nucleic acid encoding the cyclin D/CDK4 fusion protein. In certain embodiments, the kit may comprise an expression vector comprising the nucleic acid encoding a cyclin D/CDK4 fusion protein.

In particular, said kits may be configured for determining the activation status of endogenous CDK4 in eukaryotic cells or a eukaryotic cell extract. Hence, disclosed is the use of a kit for determining the activation status of endogenous CDK4 in eukaryotic cells or a eukaryotic cell extract, said kit comprising a eukaryotic cell line and a cyclin D/CDK4 fusion protein. Also disclosed is the use of a kit for determining the activation status of endogenous CDK4 in eukaryotic cells or a eukaryotic cell extract, said kit comprising a eukaryotic cell line and a nucleic acid encoding a cyclin D/CDK4 fusion protein.

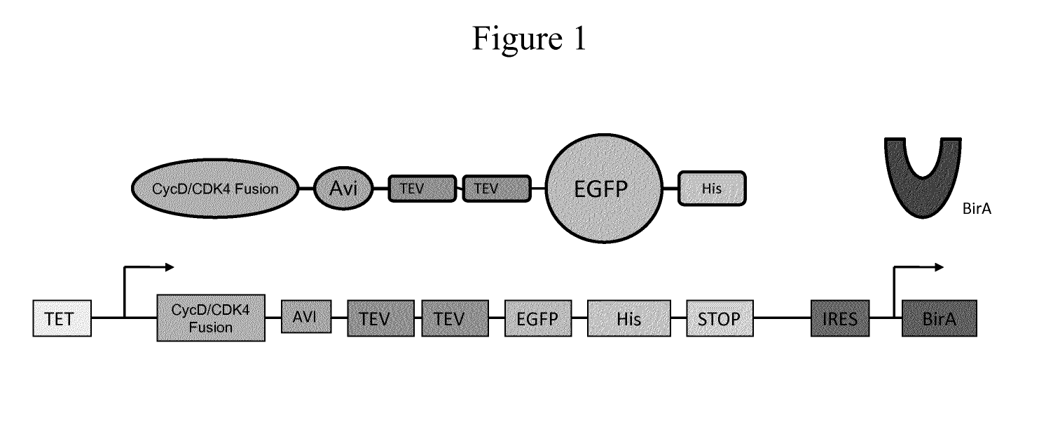

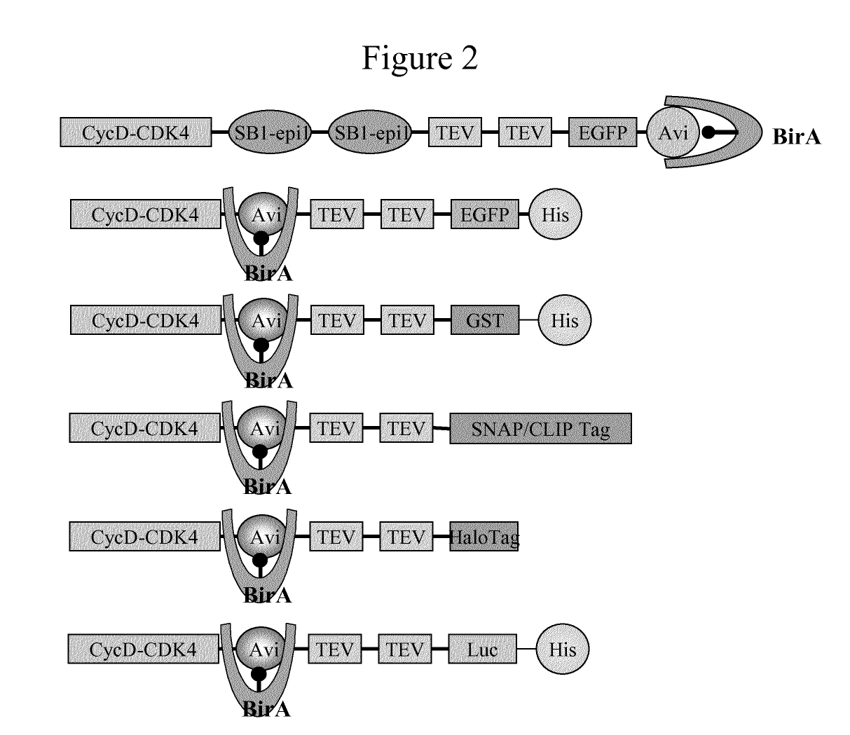

Non-limiting examples of the reporter molecule and reporter system according to different embodiments of the present invention are for instance illustrated in FIGS. 1 and 2.

Referring to FIG. 1, a reporter system according to an embodiment of the present invention is shown above comprising a reporter molecule according to an embodiment of the present invention and the biotine ligase (BirA) protein. The reporter molecule according to an embodiment of the present invention comprises a cyclin D/CDK4 fusion protein, an Avi tag, a tobacco etch virus 1 cleavage site (TEV), enhanced green fluorescent protein (EGFP), and the SB1 epitope (SBI epi). The construct expressing the reporter system according to an embodiment of the present invention is shown below in FIG. 1. The reporter molecule is expressed under the control of a tet response element.

Referring to FIG. 2, reporter systems according to different embodiments of the present invention are shown. Each reporter system illustrating the present invention comprises a reporter molecule according to an embodiment of the present invention and a BirA protein bound to the Avi tag. The reporter molecules illustrating the present invention comprise a cyclin D/CDK4 fusion protein, an Avi Tag and one or more tags selected from enhanced green fluorescent protein (EGFP), Flag, V5, Myc, HA, SB1 epitopes (SBI epi), glutathion-S-transferase (GST), SNAP/CLIP tag and Halo tag. It will be understood by the skilled person that any other tag or epitope may be used as an affinity tag such as a STREP tag, maltose binding protein (MBP), poly-histidine tag, etc. The one skilled in the art would also think to place these purification tags or modules at the N-terminus of the transgene. In the reporter molecules of the present invention the Avi-tag may be located at the C-terminus of the cyclin D/CDK4 fusion protein or the Avi-tag may be located in the construct at the C-terminus of the transgene for instance with two copies of the SB1 epitope located in front of the TEV cleavage sites as show in the upper panel of FIG. 2.

In addition, the reporter molecules as illustrated in FIG. 2 comprise the TEV tobacco etch virus cleavage site (TEV). Other consensus cleavage sites or inteins can replace the TEV sequence as appreciated by those skilled in the art.

The term “binding molecule” used herein refers to all suitable binding molecules that are specifically binding or interacting with phosphorylated CDK4 as defined by the present invention. Examples of suitable binding agents are antibodies, monoclonal- or polyclonal antibodies, nanobodies, affybodies, antibody fragments, antigen binding fragments of antibodies, aptamers, photoaptamers, spiegelmers, oligonucleotides, lipocalins, specifically interacting small molecules, Molecular Imprinting Polymers (MIPs), DARPins, ankyrins, specifically interacting proteins, peptidomimetics, biomimetics or peptides, and any other molecules that specifically bind to one of the biomarkers. Both monoclonal, polyclonal or single chain antibodies or antigen-binding antibody fragments that specifically bind the phosphorylated form of CDK4 are useful in the methods and kits of the present invention. Monoclonal and polyclonal antibodies or their antigen-binding fragments can be prepared by methods known in the art and are often commercially available. Aptamers that bind specifically to the phosphorylated form of CDK4 can be obtained using the so called SELEX or Systematic Evolution of Ligands by EXponential enrichment. In this system, multiple rounds of selection and amplification can be used to select for DNA or RNA molecules with high specificity for a target of choice, developed by Larry Gold and coworkers and described in U.S. Pat. No. 6,329,145. Recently a more refined method of designing co-called photoaptamers with even higher specificity has been described in U.S. Pat. No. 6,458,539 by the group of Larry Gold. Methods of identifying binding agents such as interacting proteins and small molecules are also known in the art. Examples are two-hybrid analysis, immunoprecipitation methods and the like.

In another aspect, the invention hence further provides assays, tools and methods for the identification of such phospho-CDK4-specific binding molecules, such as peptides or small molecules, monoclonal- or polyclonal antibodies, nanobodies, affybodies, antigen-binding antibody fragments, aptamers, photoaptamers, spiegelmers, lipocalins, specifically interacting small molecules, Molecular Imprinting Polymers (MIPs), DARPins, ankyrins, specifically interacting proteins or peptides, and other molecules that specifically bind to one of the biomarkers, using the cyclin D/CDK4 fusion proteins according to the invention (in vitro approach) or cells or cell-lines according to the invention transfected with said fusion proteins (in vivo approach).

To this end, the invention provides the use of cells and/or cell-lines that are transfected with the cyclin D/CDK4 fusion proteins according to the invention for identifying binding molecules that specifically bind the phosphorylated form of CDK4.

Furthermore, the invention provides the use of the cyclin D/CDK4 fusion proteins according to the invention for identifying binding molecules that specifically bind the phosphorylated form of CDK4.

In addition, the invention provides for a method of identifying binding agents that are specific for the phosphorylated form of CDK4, comprising the steps of comparing the binding affinity of said binding molecules to the phosphorylated fusion Cyclin D/CDK4 fusion protein, wherein said fusion protein can be phosphorylated in vitro or in vivo.

For the in vitro assay or method, the cyclin D/CDK4 fusion protein is contacted with a cell extract or either serum-activated cells or quiescent cells, and the binding of the candidate binding agent is compared in both cases. Higher binding affinity towards the serum-activated fusion protein indicates specificity for the phosphorylated form of CDK4. Ideally, the binding molecule would have no or very low affinity for the non-phosphorylated CDK4 (i.e. contacted with extract of quiescent cells) and high affinity for the phosphorylated CDK4 (i.e. the fusion protein contacted with serum-activated cell extract). Since the fusion proteins according to the present invention can be immobilized on a suitable matrix, detection and even purification of the specific binding molecules is simplified.

For the in vivo assay or method, cells are used that were stably transfected with the fusion proteins according to the present invention. The fusion protein can be extracted from these cells through the affinity tags in the plasmids. Comparing the binding affinity towards the isolated fusion proteins obtained from cultures of cells that were treated with serum or were left quiescent will again enable the identification and characterization of those binding molecules that are specific for the phosphorylated CDK4 isoform.

The invention further provides kits for identifying new binding molecules that specifically bind phosphorylated CDK4 or cells expressing phosphorylated CDK4, comprising the fusion proteins or cell-lines transfected with said cyclin D/CDK4 fusion proteins. In addition, said kits can also comprise the necessary means to activate cells or to leave them in a quiescent state.

The aspects of the invention mentioned above will now be demonstrated in the following non-limiting examples.

### EXAMPLES

### Example 1

### Assays to Determine the Activation Status of CDK4

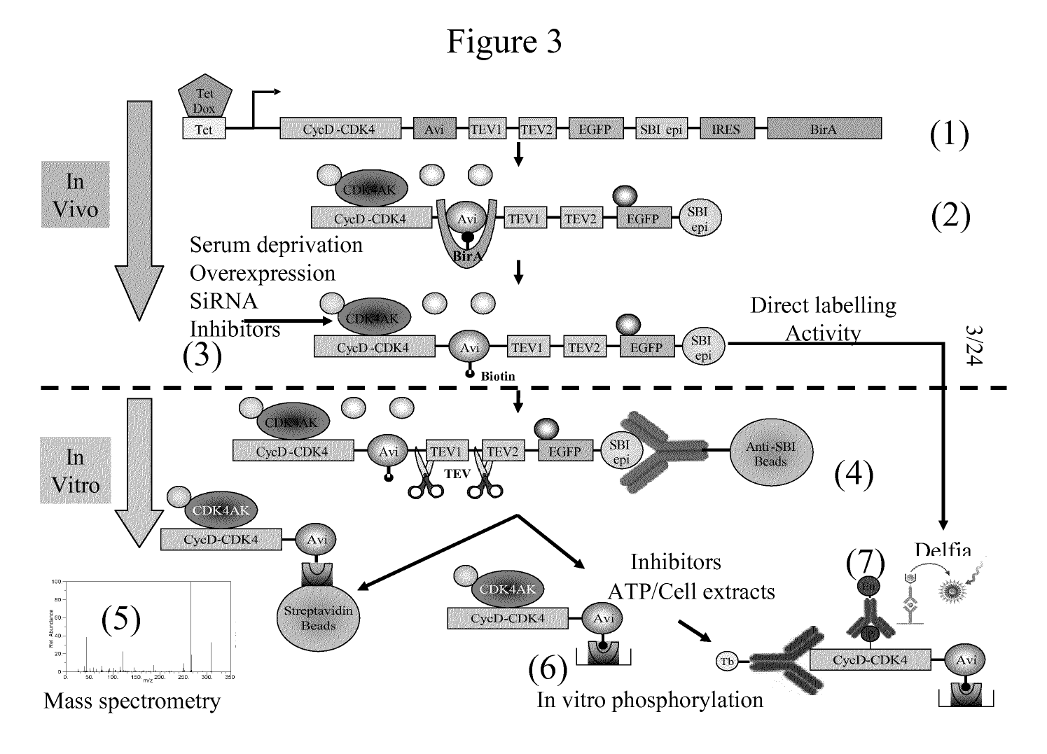

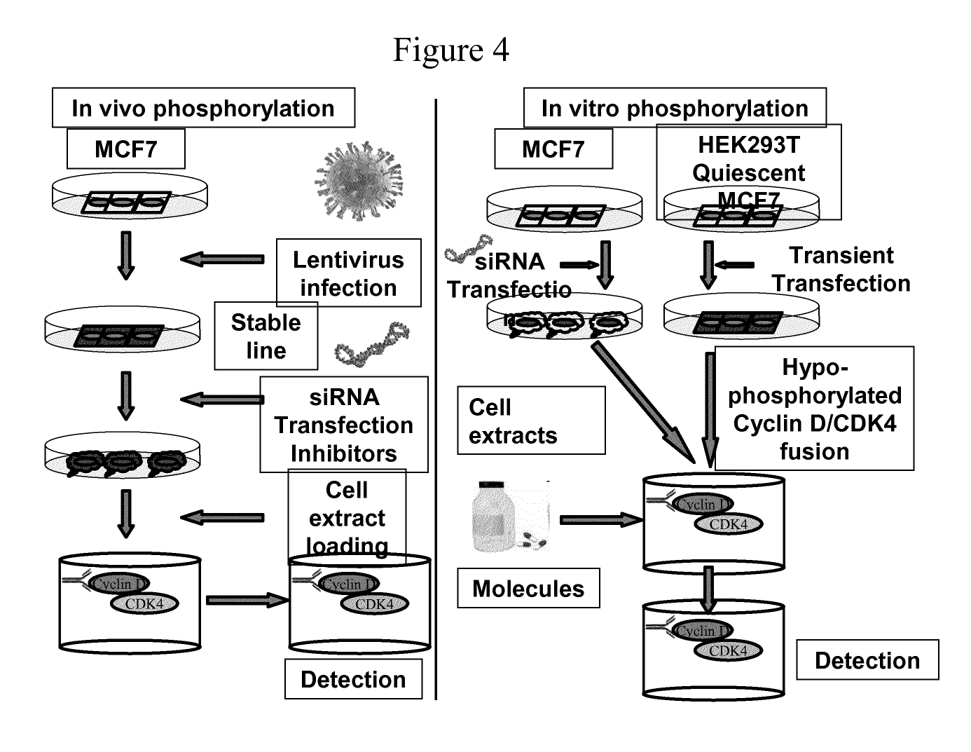

In a first configuration of an assay according to an embodiment of the present invention (illustrated in FIG. 3 and in the left panel of FIG. 4), phosphorylation of the CDK4 part of the cyclin D/CDK4 fusion protein occurs in intact cells, transfected with a nucleic acid encoding a reporter molecule comprising the cyclin D/CDK4 fusion protein.

In the second configuration of an assay according to an embodiment of the present invention (illustrated in the right panel of FIG. 4), phosphorylation of the CDK4 part of the cyclin D/CDK4 fusion protein occurs in vitro, in particular in cellular extract. In this case, the cyclin D/CDK4 fusion isolated in a hypo-phosphorylated state from quiescent MCF7 cells stably expressing the cyclin D/CDK4 fusion is immobilized on streptavidin-coated matrix. Alternatively, the cyclin D/CDK4 fusion can be isolated from HEK293T cells transiently transfected with an expression vector for the cyclin D/CDK4 fusion or from proliferating MCF7 cells stably expressing the cyclin D/CDK4 fusion, immobilized on streptavidin-coated matrix and dephosphorylated by a phosphatase such as, for example, the λ-phosphatase. Next, the immobilized hypophosphorylated cyclin D/CDK4 fusion protein is incubated with cellular extracts or purified kinases together with ATP and optionally molecules under investigation before the Rb kinase activity of the immobilized cyclin D/CDK4 fusion protein, or its activation state will be measured as described herein. Cellular extract will be prepared from cells overexpressing specific kinases or in which expression of a specific kinase had previously been decreased by siRNA or with any other suitable technology known to those skilled in the art.

The choice between the two assay configurations will be dictated by the compatibility between the time course of the regulation of the CDK4 phosphorylation and the time course of the tested biological perturbation, e.g. siRNA transfection, kinase overexpression, or addition of a chemical compound.

### Example 2

### Screening of siRNA Library to Identify CDK4 Activating Kinases

Two examples of the assays according to certain embodiments of the present invention are described in detail. In a first example (shown in the left panel of FIG. 4), the assay or method is based on an in vivo regulation of phosphorylation of CDK4. The assay is based on the generation of stable eukaryotic cell lines using retrovirus. Retroviruses can insert a plasmid construct into the genome of the eukaryotic cells. Subsequently, a siRNA directed against a target activating kinase, for instance a proline directed kinase (PDK) is introduced into the eukaryotic cells upon inducing proliferation of the eukaryotic cells. Next, the reporter molecule is isolated from the eukaryotic cells and the activation status of CDK4 measured using for instance the DELFIA system.

In the second example (shown in the right panel of FIG. 4), the assay or method is based on an in vitro regulation of phosphorylation of CDK4. The plasmid constructs is transiently transfected in HEK293T cells. The cyclin D/CDK4 fusion protein is extracted and purified on wells covered with streptavidin. In parallel, a siRNA directed against a target activating kinase, for instance a proline directed kinase (PDK), is introduced in a eukaryotic cell line. The protein lysate of this eukaryotic cell line is recovered and brought onto the reporter molecule comprising the cyclin D/CDK4 fusion protein. The activation status of CDK4 is then measured for instance with the DELFIA system.

### Example 3

### Screening of Inhibitory Compounds of cyclinD/CDK4 Complexes to Identify Anti-Cancer Drugs or Drugs Effective Against Proliferative Diseases

A reporter molecule comprising a cyclin D/CDK4 fusion protein is produced in a eukaryotic cell line by culturing the cells while they are kept in a quiescent state, thereby producing the cyclin D/CDK4 fusion protein wherein CDK4 is present in a hypophosphorylated form. Subsequently, a compound of interest is added to the eukaryotic cells upon inducing proliferation of the eukaryotic cells. Next, the reporter molecule is isolated from the eukaryotic cells and the activation status of CDK4 measured using for instance the DELFIA system.

In the case where the compound is an inhibitory compound of the upstream pathway of the cyclin D/CDK4 complex, for instance if the compound is directed against a CDK4 activating kinase or blocks cyclin D/CDK4 phosphorylation, no substrate phosphorylation is present in the wells.

### Example 4

### Comparison of the Reporter Molecule Comprising a Cyclin D3/CDK4 Fusion Protein with Other Reporter Molecules

Experiments are performed to test whether cyclin D1/CDK4 or cyclin D2/CDK4 fusion proteins coupled to an Avi-tag and expressed together with BirA/mCherry or BirA/puro fusion bear an immobilisable serum-modulated Rb kinase activity. Similar experiments are performed with fusions in which the T172 and the P173 of CDK4 are mutated to alanine or serine, respectively. Similar experiments are performed with cyclin D1/CDK6, cyclin D2/CDK6, and/or cyclin D3/CDK6 fusion proteins coupled to an Avi-tag and expressed together with BirA/mCherry or BirA/puro fusion protein. Finally, the experiments described above are repeated with CDK4 or CDK6 alone coupled to an Avi-tag and expressed together with BirA/mCherry or BirA/puro fusion.

### Example 5

### Measurement of the Activation Status of CDK4 with Anti-Phospho-CDK4 or Anti-Phospho-TP Antibodies or with Nanobodies

Direct quantification of CDK4 phosphorylation is performed with anti-phospho-CDK4 or anti-phospho-TP antibodies or with nanobodies selected to recognize specifically either the non-phosphorylated or the phosphorylated state of the CDK4.

The Avi-tag is initially located at the C-terminus of the fusion proteins as described in (Schäffer et al., supra). Another set of constructs with the Avi-tag located after the CDK4 sequence in front of a TEV cleavage site placed before an EGFP or a GST sequence is generated. If biotinylation of the Avi-tag located within the fusion protein is as efficient as the biotinylation of the Avi-tag located at the C-terminus of the fusion, tandem purification of the cyclin D/CDK4 fusions is possible. After immobilization of the fusion proteins with immobilized anti-EGFP, anti-SB1 antibodies or glutathion-coupled supports, the biotinylated Avi-tagged cyclin D/CDK4 moiety of the fusion proteins will be released by digestion with the TEV protease. In case the linker between the cyclin D and CDK4 bears a Prescission protease cleavage site, both cyclin D and biotinylated avi-tagged CDK4 part could be further dissociated by treatment with the Prescission protease. After thermic denaturation which will probably disrupt most protein/protein interaction only the biotinylated CDK4 fragment of the cyclin D/CDK4 fusion protein will be bound to streptavidin immobilized on a solid matrix. Phosphorylation of this peptide can be detected by anti-phospho-CDK4 or anti-phospho-TP antibodies or with nanobodies or aptamers. Alternatively, the phosphorylation status of tryptic fragments of the CDK4 fragment of the cyclin D/CDK4 fusion proteins immobilized on streptavidin can be quantified directly by mass spectrometry.

### Example 6

### Characterization of Phosphorylation State-Specific Interaction Partners of Endogenous Cyclin D/CDK4 Complexes

The activating phosphorylation of CDK2 induces a dramatic change in its conformation. The impact of the T172 phosphorylation on the conformation of CDK4 is less severe and not as well characterized. Binding of ATP, ATP analogues, appropriately designed pseudo-substrates, or modified p21 or p27 proteins is determined according to the phosphorylation status of the cyclin D/CDK4 fusion protein. To identify phosphorylation status-dependent cyclin D/CDK4 fusion protein interaction partners, the composition of isolated cyclin D/CDK4 fusion protein complexes is compared between quiescent and serum stimulated cells at different times of the cell cycle. Comparative mass spectrometry methods such as SILAC or COFRADIC, or comparative 2D gel electrophoresis methods such as 2D-DIGE, or other techniques known to those skilled in the art such as derivatives of two hybrid technologies, can be used in this matter. In case a phosphorylation state-specific interaction partner of the cyclin D/CDK4 fusions is identified, its interaction domain is determined and fused to reporter proteins such as luciferase, peroxidises, or fluorescent proteins to quantify its binding to the cyclin D/CDK4 fusion protein. Binding of the phosphorylation state-specific interaction partner of the cyclin D/CDK4 fusion protein can also be quantitated using specific antibodies, nanobodies or aptamers.

### Example 7

### Screening Assay to Determine the Activation of CDK4

A preferred setup of the assay is depicted in FIG. 3. In this configuration, a reporter molecule is stably incorporated in the genome of a mammalian cell model suitable to follow the CDK4 activation such as MCF7 cells. This reporter molecule can be under the control of a constitutive promoter or inducible promoter. FIG. 3 illustrates the situation where the transgene expression is driven by a Tet On system. When cells express the Tet activator, addition of doxycyclin to the cells induces a conformational change in the Tet regulator which is then able to bind to a specific sequence added in front of the transgene expression module. This binding activates transcription of the reporter molecule (FIG. 3, step 1). This reporter molecule comprises the sequence of a cyclin D/CDK4 fusion protein linked to an Avi tag. The Avi tag encodes for a consensus peptide of 13 amino acids recognized by the biotin ligase of E. Coli and biotinylated by this enzyme (FIG. 3, step 2). The BirA enzyme is expressed together with the fusion protein via an IRES sequence cloned between the nucleic acid sequence encoding the fusion protein and the nucleic acid sequence encoding the BirA biotin ligase. The IRES sequence when present in a mRNA can bind to ribosomes and allows them to start translation at this site, albeit with a low efficiency. In another configuration, constitutive or inducible expression of the BirA biotin ligase can be supported by another locus. As illustrated in FIG. 3, the Avi tag coupled to the cyclin D/CDK4 fusion protein may be followed by two TEV cleavage sites recognized and cut by the TEV protease. The TEV sites may be coupled to an EGFP sequence to allow easy identification and tracing of cells expressing the transgene. The EGFP sequence may be used to purify the transgene on Protein-A sepharose beads loaded with an anti-EGFP antibody. The EGFP sequence may be followed at its C-terminus by an SB1 epitope which can be recognized by a specific monoclonal antibody. The SB1 epitope can eventually be used to purify the transgene on Protein-A sepharose beads loaded with an anti-SB1 antibody as described previously (Schäffer et al., supra). Alternatively, the Avi-tag may be located in the construct at the C-terminus of the transgene while two copies of the SB1 epitope are located in front of the TEV cleavage site as show in FIG. 2, upper panel. The SB1 epitope can eventually be used to purify the transgene on Protein-A sepharose beads loaded with an anti-SB1 antibody as described previously (Schäffer et al., supra). Alternatively, the Avi-tag may be located in the construct at the C-terminus of the transgene while two copies of the SB1 epitope are located in front of the TEV cleavage site as show in FIG. 2, upper panel.

Advantageously, the SB1 epitope can be replaced by an other purification tag as known to those skilled in the art as exemplified in FIG. 2.

Cells expressing the cyclin D/CDK4 fusion protein tagged with the Avi-tag and expressing the BirA biotin ligase are then induced to proliferate while specific genes (especially proline-directed kinases) are overexpressed or knocked down by siRNA silencing or any other methods known to those skilled in the art (FIG. 3, step 2). Proliferation can also be induced together with treatment of the cells with chemicals under investigation (FIG. 3, step 2). At the end of these treatments, cells are lysed in non denaturing condition (FIG. 3, step 3) before the fusion proteins are purified by the appropriate purification technique. In the example illustrated in FIG. 3, fusion proteins are first purified on a protein-A sepharose matrix loaded with anti-SB1 antibody and eventually released by cleavage with the TEV protease (FIG. 3, step 4). The cyclinD/CDK4 fusion protein can then be purified on immobilized streptavidin before further analysis by mass spectroscopy (FIG. 3, step 5), Rb kinase assay (FIG. 3, step 6), or immunodetection with the Delfia technology (FIG. 3, step 7), or other methods known to those skilled in the art.

The cyclin D and the CDK4 moieties of the cyclin D/CDK4 fusion protein can be separated by a linker including a protease sensitive cleavage site. Proteolytic dissociation of the cyclin D/CDK4 fusion protein followed by thermic denaturation of the lysate will allow specific purification of the CDK4 moiety by immobilized streptavidin. The phosphorylation status of this peptide can subsequently be determined using any anti-phospho-CDK4 or any phospho-TP antibody as well as by mass spectroscopy or any other relevant technique known to those skilled in the art. Presence of the anti-phospho-CDK4 or the phospho-TP antibody on the immobilized CDK4 part of the fusion could be detected by the DELFIA technology as illustrated in FIG. 3, or by radioactive, colorimetric or chemiluminescence methods known to those skilled in the art.

### Example 8

### Generation of Lentiviral Expression Vectors to Record the Activation of CDK4 Upon Induction of Proliferation

In order to create stable cell lines expressing any transgene of interest, a lentiviral expression system was generated to express transgenes marked with a biotinylable tag, the Avi tag. Indeed, when biotinylated by a humanized version of the BirA biotin ligase of E. coli, the Avi tag allows easy purification of the proteins to which it is coupled to by streptavindin coupled supports as described previously (Schäffer et al., 2009, Nucleic acid res., 38, 1-13).

Different building blocks of the reporter molecule according to certain embodiments of the present invention are illustrated in Table 1.

TABLE 1
Building blocks of the reporter molecule according
to certain embodiments of the present invention

| Building block | SEQ ID NO. |
|---|---|
| Cyclin D1 | 1 |
| CDK4 | 2 |
| EGFP-Avi | 11 |
| TEV | 12 |
| SB1 | 13 |
| Avi tag | 14 |

| | Nucleotide | Amino acid |
|---|---|---|
| | sequence | sequence |
| BirA/mCherry fusion | 15 | 16 |
| BirA/puromycin resistance | 17 | 18 |
| gene fusion | | |
| Expression module 1 | 19 | — |
| Expression module 2 | 20 | — |
| Expression module 3 | 21 | — |
| D3L3K4 | 78 | 79 |
| D1L3K4 | 80 | 81 |

The different plasmids used in the construction of the reporter molecule as used in the assay of the present invention and of control constructs such as those comprising the luciferase gene, are listed in Table 2.

TABLE 2
Plasmids used in the construction of the reporter molecule as used
in the assay according to an embodiment of the present invention
and plasmids used in the construction of control constructs

| Plasmid | SEQ ID No. |
|---|---|
| pDLVCTEGFPtetOV5His_luc | 22 |
| pENTRtopo_D1 | 23 |
| pENTRtopo_D1L1K4 | 24 |
| pENTRtopo_D1L2K4 | 25 |
| pENTRtopo_D3L1K4 | 26 |
| pENTRtopo_D3L2K4 | 27 |
| pENTRtopo_D1L3K4 | 82 |

The pDLVCTEGFPTetOV5HisLuc vector used as positive control for non-biotinylable luciferase was obtained at the University of Ghent by recombination of the pDLVCTEGFPTetOV5His destination vector with the pENTRDTopo-Luc using the LR clonase mix of Invitrogen according to the instruction of the manufacturer. The former is a derivative of the pWPI vector (Addgene #12254) described by the team of Pr. Trono (University of Lausanne, Switzerland) (Wiznerovicz et al., 2003, J. Virol., 77, 8957-8961) in which an expression module 1 (SEQ ID NO. 19) was inserted between PpuMI and NdeI. The expression module comprised a tet response element followed by the CMV promoter in front of a Gateway recombination module coupled in frame to a V5His tag and an internal ribosome entry site (IRES) sequence driving the expression of the fluorescent protein EGFP. The Luciferase entry vector pENTRDTopo-Luc was created at the University of Ghent by amplifying the luciferase reporter gene from the pGL4 plasmid (Promega) by PCR and cloning the corresponding fragment in the pENTRDTopo vector.

The inventors transferred the EGFP-Avi tag together with a Gateway recombination module from the vector pBY2807 acquired via Addgene (Addgene plasmid #23222) described previously (Schäffer et al., supra) in the vector pLVTH/KrabRed (Addgene plasmid #11643) described by the team of Pr. Trono (University of Lausanne, Switzerland) (Wiznerovicz et al., supra). They also included a tet response element followed by the EF1α promoter in front of a Gateway recombination module and an internal ribosome entry site (IRES) sequence driving the expression of the BirA/mCherry fusion excised from the pBY2982 plasmid acquired via Addgene (Addgene plasmid #23220) described previously (Schäffer et al., supra). This module, i.e., expression module 2 (SEQ ID NO. 20), was inserted between the PpuMI and NdeI sites of the pLVTH/KrabRed vector to create the destination vector pWFAviIBc. Another module, i.e., expression module 3 (SEQ ID NO. 21), including the BirA/puromycin resistance gene fusion was inserted between the PpuMI and KpnI sites of the pLVTH/KrabRed vector to create the destination vector pWFAviIBp. The BirA/puromycin resistance gene fusion was obtained by combining the BirA sequence of the pBY2982 plasmid and the puromycin resistance gene through a NcoI site. As appreciated by those in the art, numerous strategies involving PCR or not can be followed to achieve these constructs according to protocols well known in the field. Recombinant DNA was transformed in DB3.1 bacteria. Clones transformed with the expected plasmid were selected according to the restriction profile obtained after the digestion of their plasmid DNA with NcoI.

The inventors transferred the BirA coding sequence of the pBY2982 plasmid acquired via Addgene (Addgene plasmid #23220) described previously (Schäffer et al., supra) to the the vector pLVTH/KrabRed (Addgene plasmid #11643) described by the team of Pr. Trono (University of Lausanne, Switzerland) (Wiznerovicz et al., supra) by digesting the former with SmaI and MscI and inserting the BirA coding fragment in the latter cut by SmaI and PmeI. The DsRED selection marker of the resulting plasmid was further replaced by a neomycin resistance gene through its KpnI/KpnI digestion and ligation with a KpnI/KpnI DNA fragment isolated from the pLVINeo plasmid. This plasmid was obtained by transferring the IRES sequence followed by the Neomycin resistance gene cut by BamHI/XbaI from the pIRESNeo plasmid from Clontech (Genbank #U89673) in the pLVET-tTR-KRAB vector (Addgene plasmid #11644) described by the team of Pr. Trono (University of Lausanne, Switzerland) (Wiznerovicz et al., supra) cut by BamHI and SpeI.

The inventor also created a lentiviral vector to express the rTTA3 doxycyclin-sensitive regulator under the control of the strong EF1α eukaryotic promoter together with the DsRED fluorescence marker under the control of and IRES sequence. To this end, the pDG2iV5puro vector created at the University of Ghent and described previously (Bisteau et al, PLoS Genet. 2013 May; 9(5):e1003546) was first cut by XbaI and re-ligated. The puromycin selection marker from this plasmid was then replaced by a DsRED selection marker through its KpnI/KpnI digestion and ligation with a KpnI/KpnI DNA fragment isolated from the pLVTH/KrabRed plasmid (Addgene plasmid #11643) to create the pDG2rTTA3IDR plasmid. The rTTA3 regulator was finally inserted downstream of the EF1α promoter by transferring the MluI/SpeI fragment of the pDG2rTTA3IDR plasmid including the rTTA3 regulator in the pLVTH/KrabRed plasmid (Addgene plasmid #11643) cut by MluI and SpeI to generate the vector pLVrTTA3IDRm (SEQ ID No. 67).

The inventors generated a lentiviral expression vector called pWNV5His allowing the induction by doxycyclin of the expression of a transgene fused to the V5His tag by inserting a PpumI/XmaI fragment of the pDG2iV5puro vector created at the University of Ghent and described previously (Bisteau et al, PLoS Genet. 2013 May; 9(5):e1003546) into the PpumI/XmaI-cut pDLVCTEGFPTetOV5His vector described above.

The inventors also generated lentiviral expression vectors allowing the induction by doxycyclin of the expression of a fusion protein containing the Avi-tag separated from a poly-histidine purification tag by two TEV cleavage sites and eventually a GFP sequence. The vector including the Avi-tag, the TEV sites, the GFP sequence and the poly-histidine purification tag was generated first by PCR amplification of part of the Gateway cassette from the vector pDLVCTEGFPTetOV5His described above with the primers ESR46 and ESR47 described below. Next the GFP sequence preceded by TEV sites was amplified by PCR from the pWFAviIBc DNA with the primers ESR50 and ESR51 described below. The combined Avi-tag and TEV sites sequences were generated by annealing the ESR49 and ESR48 primers and extending the annealed DNA by PCR amplification. The Gateway cassette amplicon was combined with the resulting Avi-tag-TEV sequence and amplified by PCR with the primers ESR46 and ESR49. The TEV-GFP amplicon was combined with the resulting Avi-tag-TEV sequence and amplified by PCR with the primers ESR48 and ESR51. These two amplicons were combined with the pWNV5His vector cut with MluI and XmaI using the Gibson assembly Mix (NEB) to create the pWNATGHis vector. The pWNATHis vector lacking the GFP sequence between the TEV sites and the poly-histidine tag was created by amplifying the Gateway cassette fragment followed by the Avi-tag and two TEV sites from the pWNATGHis vector with the primers ESR46 and ESR52 and combining the corresponding amplicon with the pWNV5His vector cut with MluI and XmaI using the Gibson assembly Mix (NEB).

The pWFAviIBc, pWFAviIBp, pWNATGHis and pWNATHis were recombined with entry vectors (pENTRtopo vectors) comprising the expression modules for luciferase (pENTRtopo_Luc), cyclin D1 (pENTRtopo_D1), CDK4 (pENTRtopo_K4), cyclin D1/CDK4 fusion protein (pENTRtopo_D3LxK4, with x being 1, 2 or 3 depending on the linker used), or cyclin D3/CDK4 fusion protein (pENTRtopo_D3LxK4, with x being 1, 2 or 3 depending on the linker used) using the LR clonase mix of Invitrogen according to the instruction of the manufacturer (Gateway system recombination). Recombinant DNA was transformed in Top10 bacteria. Clones transformed with the expected plasmid were selected according to the restriction profile obtained after the digestion of their plasmid DNA with NcoI. Complete sequences of the resulting plasmids can be found in the sequence listing: pWNATGHis_Luc (SEQ ID No. 69); pWNATGHis_D3L3K4 (SEQ ID No. 68); pWNATHis_Luc (SEQ ID No. 71); pWNATHis_D3L3K4 (SEQ ID No. 70). The sequence Linker 3 (L3) can be found in SEQ ID No. 72 (DNA) and 73 (amino acid).

Entry vectors for cyclin D1, CDK4, cyclin D1/CDK4 fusion protein or cyclin D3/CDK4 fusion protein were created by amplifying the corresponding sequences with the PFX polymerase and cloning the respective inserts in the pENTRDTopo vector according to the instruction of the manufacturer (Invitrogen). Fusion protein entry vectors were created by a multistep PCR strategy. Both the cyclin D with a part of the linker and the CDK4 with a part of the linker partially overlapping the former were separately amplified by PCR. The PCR products were next mixed in a new PCR reaction including the cyclin D forward primer and the CDK4 reverse primer. The sequences of the primers used to this end are described in Table 3. The PCR primers and conditions used to generate the different inserts are given in Tables 4 and 5 respectively.

The PCR reaction mix included 5 μl PFX Buffer, 5 μl PFX Enhancer Buffer, 1.5 μl dNTP Mix (10 mM for each), 1 μl MgSO₄ 50 mM, 1 μl Primer 1 (Table 4), 1 μl Primer 2 (Table 4), 1μ Primer 3 (Table 4, when indicated), all at 200 ng/μl, 1 μl of DNA template 1 (Table 5), 1 μl DNA template 2 (Table 5, when indicated) both at 100 ng/μl, 1 μl PFX polymerase brought to a final volume of 50 μl with autoclaved ultapure DNAse-free water. Amplification was started by incubating the PCR mix for 2 minutes at 94° C. This incubation was followed with 30 cycles of incubation at 94° C. for 15 seconds, incubation at the indicated temperature (Table 5, hybridization temperature) for 30 seconds, and for the indicated time (Table 5, elongation time) at 68° C. The PCR reaction was ended by incubation for 7 minutes at 68° C. and stored afterwards at 4° C.

Entry vectors including the non-activable T172A mutant of CDK4 were generated by site-directed mutagenesis of the corresponding wild type constructs using the QuikChange mutagenesis kit (Stratagene) and the primers 4153CDK4T172A_QC_F (SEQ ID No. 76) and 4153CDK4T172A_QC_R (SEQ ID No. 77).

TABLE 3
Sequences of primers used for the amplification of cyclin D1, CDK4, cyclin
D1/CDK4 fusion protein, and cyclin D3/CDK4 fusion protein

| Primer | Sequence | SEQ ID NO. |
|---|---|---|
| 4153FusCycD1_F | CACCATGGAACACCAGCTCCTGTGC | 28 |
| 4153FusCDK4_R | TCAGATGTCCACGTCCCGCAC | 29 |
| 4153FusCycD3_F | CACCATGGAGCTGCTGTGTTGCGAA | 30 |
| 4153FusCycD3_R1 | GCTGCCGCCGCCGCCGCTGCCGCCGCCGCCGCTGCCGCCGCCG | 31 |
| | CCCTTGCTGGCCAGGTGTATGGCTGTGACATCT | |
| 4153FusCycD3_R1c | GCTGCCGCCGCCGCCGCT | 32 |
| 4153FusCycD3_R1′ | CTGCCGCCGCCGCCGCTGCCGCCGCCGCCCTTGCTGGCCAGGT | 33 |
| | GTATGGCTGTGACATCT | |
| 4153FusCycD3_R1′c | CTGCCGCCGCCGCCGCTG | 34 |
| 4153FusCycD3_R2 | GCGGCTGGGCTGGAACAGCACCTCCAGGCTGCCGCCGCCGCCC | 35 |
| | TTGCTGGCCAGGTGTATGGCTGTGACATCT | |
| 4153FusCycD3_R2c | GCGGCTGGGCTGGAACAG | 36 |
| 4153FusCycD3_R2′ | CAGCACCTCCAGGCTGCCGCCGCCGCCCTTGCTGGCCAGGTGT | 37 |
| | ATGGCTGTGACATCT | |
| 4153FusCycD3_R2′c | CAGCACCTCCAGGCTGCC | 38 |
| 4153FusCDK4_F1 | GCCAGCAAGGGCGGCGGCGGCAGCGGCGGCGGCGGCAGCGGCG | 39 |
| | GCGGCGGCAGCATGGCTACCTCTCGATATGAG | |
| 4153FusCDK4_F1c | GCCAGCAAGGGCGGCGGCGGC | 40 |
| 4153FusCDK4_F1′ | GGCAGCGGCGGCGGCGGCAGCGGCGGCGGCGGCAGCATGGCTA | 41 |
| | CCTCTCGATATGAG | |
| 4153FusCDK4_F1′c | GGCAGCGGCGGCGGCGGCAGC | 42 |
| 4153FusCDK4_F2 | GCCAGCAAGGGCGGCGGCGGCAGCCTGGAGGTGCTGTTCCAGC | 43 |
| | CCAGCCGCATGGCTACCTCTCGATATGAG | |
| 4153FusCDK4_F2c | GCCAGCAAGGGCGGCGGC | 44 |
| 4153FusCDK4_F2′ | GGCGGCAGCCTGGAGGTGCTGTTCCAGCCCAGCCGC ATGGCT | 45 |
| | ACCTCTCGATATGAG | |
| 4153FusCDK4_F2′c | GGCGGCAGCCTGGAGGTG | 46 |
| 4153FusCycD1-F1 | CACCATGGAACACCAGCTCCTGTG | 47 |
| 4153FusCycD1_R1 | GCTGCCGCCGCCGCCGCTGCCGCCGCCGCCGCTGCCGCCGCCG | 48 |
| | CCCTTGCTGGCGATGTCCACGTCCCGCACGTC | |
| 4153FusCycD1_R1′ | CTGCCGCCGCCGCCGCTGCCGCCGCCGCCCTTGCTGGCGATGT | 49 |
| | CCACGTCCCGCACGTC | |
| 4153FusCycD1_R2 | GCGGCTGGGCTGGAACAGCACCTCCAGGCTGCCGCCGCCGCCC | 50 |
| | TTGCTGGCGATGTCCACGTCCCGCACGTC | |
| 4153FusCycD1_R2′ | CAGCACCTCCAGGCTGCCGCCGCCGCCCTTGCTGGCGATGTCC | 51 |
| | ACGTCCCGCACGTC | |
| 4153CycD3top_RaSTOP | CAGGTGTATGGCTGTGACATCT | 52 |
| 4153CycD1top_F | CACCATGGAACACCAGCTCCTGTGC | 53 |
| 4153CycD1Top_RaSTOP | GATGTCCACGTCCCGCACGTC | 54 |
| 4153CycD1Top_R | TCAGATGTCCACGTCCCGCAC | 55 |
| 4153FusK4_R_aStop | CTCCGGATTACCTTCATCCTT | 56 |
| 4153CDK6_R_aStop | GGCTGTATTCAGCTCCGAGGT | 57 |
| 4153FusCDK4_F2jc | GCCAGCAAGGGCGGCGGC | 58 |
| 4153FusCycD3_R2jc | GCGGCTGGGGCCCTGGAA | 59 |
| 4153FusCDK6_F1 | GCCAGCAAGGGCGGCGGCGGCAGCGGCGGCGGCGGCAGCGGCGG | 60 |
| | CGGCGGCAGCATGGAGAAGGACGGCCTGTGC | |
| 4153FusCDK6_F1′ | GGCAGCGGCGGCGGCGGCAGCGGCGGCGGCGGCAGCATGGAGAA | 61 |
| | GGACGGCCTGTGC | |
| 4153FusCDK6_F2 | GCCAGCAAGGGCGGCGGCGGCAGCCTGGAGGTGCTGTTCCAGGG | 62 |
| | CCCCAGCCGCATGGAGAAGGACGGCCTGTGC | |
| 4153FusCDK4_F2j | GCCAGCAAGGGCGGCGGCGGCAGCCTGGAGGTGCTGTTCCAGGG | 63 |
| | CCCCAGCCGCATGGCTACCTCTCGATATGAG | |
| 4153FusCycD1_R2j | GCGGCTGGGGCCCTGGAACAGCACCTCCAGGCTGCCGCCGCCGC | 64 |
| | CCTTGCTGGCGATGTCCACGTCCCGCACGTC | |
| 4153FusCycD3_R2j | GCGGCTGGGGCCCTGGAACAGCACCTCCAGGCTGCCGCCGCCGC | 65 |
| | CCTTGCTGGCCAGGTGTATGGCTGTGACATCT | |
| 4153CDK4T172A_QC_F | acagctaccagatggcacttgcacccgtggtt | 32 |
| 4153CDK4T172A_QC_R | aaccacgggtgcaagtgccatctggtagctgt | 32 |
| ESR46 | GTACAGAGTGATATTATTGACACGCC | 26 |
| ESR47 | TCAACCACTTTGTACAAGAAAGCTGAACG | 29 |
| ESR48 | TACAAAGTGGTTGAGGGCCTGAACGACATCTTCGAGGCCC | 40 |
| ESR49 | CTCGTGCCACTCGATCTTCTGGGCCTCGAAGATGTC | 36 |
| ESR50 | GATCGAGTGGCACGAGGAGAACCTTTACTTTCAAGG | 36 |
| ESR51 | ATGATGACCGGTACGCGTACCACCCTCACCCTGTGCTGCC | 40 |
| ESR52 | ATGATGACCGGTACGCGTTCCCTGGAAATAGAGATTTTCC | 40 |

TABLE 4
PCR primers used to generate the different inserts

| | Primer 1 | Primer 2 | Primer 3 |
|---|---|---|---|
| Cyclin D1 wt Linker 1 | 4153FusCycD1_F | 4153FusCycD1_R1/ | 4153FusCycD3_R1c/ |
| | | 4153FusCycD1_R1′ | 4153FusCycD3_R1′c |
| Cyclin D1 wt Linker 2 | 4153FusCycD1_F | 4153FusCycD1_R2/ | 4153FusCycD3_R2c/ |
| | | 4153FusCycD1_R2′ | 4153FusCycD3_R2′c |
| Cyclin D1 wt Linker 3 | 4153FusCycD1_F | 4153FusCycD1_R2j | 4153FusCycD3_R2jc |
| Cyclin D3 wt Linker 1 | 4153FusCycD3_F | 4153FusCycD3_R1/ | 4153FusCycD3_R1c/ |
| | | 4153FusCycD3_R1′ | 4153FusCycD3_R1′c |
| Cyclin D3 wt Linker 2 | 4153FusCycD3_F | 4153FusCycD3_R2/ | 4153FusCycD3_R2c/ |
| | | 4153FusCycD3_R2′ | 4153FusCycD3_R2′c |
| Cyclin D3 wt Linker 3 | 4153FusCycD3_F | 4153FusCycD3_R2j | 4153FusCycD3_R2jc |
| CDK4 wt Linker 1 | 4153FusCDK4_F1/ | 4153FusCDK4_F1c/ | 4153FusK4_R_aStop |
| | 4153FusCDK4_F1′ | 4153FusCDK4_F1′c | |
| CDK4 wt Linker 2 | 4153FusCDK4_F2/ | 4153FusCDK4_F2c/ | 4153FusK4_R_aStop |
| | 4153FusCDK4_F2′ | 4153FusCDK4_F2′c | |
| CDK4 wt Linker 3 | 4153FusCDK4_F2j | 4153FusCDK4_F2jc | 4153FusK4_R_aStop |
| Fusion Cyclin D1 - | 4153FusCycD1_F | 4153FusK4_R_aStop | / |
| Linker 1 - CDK4 wt | | | |
| Fusion Cyclin D1 - | 4153FusCycD1_F | 4153FusK4_R_aStop | / |
| Linker 2 - CDK4 wt | | | |
| Fusion Cyclin D1 - | 4153FusCycD1_F | 4153FusK4_R_aStop | / |
| Linker 3 - CDK4 wt | | | |
| Fusion Cyclin D3 - | 4153FusCycD3_F | 4153FusK4_R_aStop | / |
| Linker 1 - CDK4 wt | | | |
| Fusion Cyclin D3 - | 4153FusCycD3_F | 4153FusK4_R_aStop | / |
| Linker 2 - CDK4 wt | | | |
| Fusion Cyclin D3 - | 4153FusCycD3_F | 4153FusK4_R_aStop | / |
| Linker 3 - CDK4 wt | | | |

TABLE 5
PCR conditions used to generate the different inserts

| | DNA | DNA | Hybridization | Elongation | |
|---|---|---|---|---|---|
| | source 1 | Source 2 | Temperature | time | |
| Cyclin D1 wt Linker 1 | pCS2-Cyclin D1flag | / | 56° C. | 1 | minute |
| Cyclin D1 wt Linker 2 | pCS2-Cyclin D1flag | / | 56° C. | 1 | minute |
| Cyclin D1 wt Linker 3 | pCS2-Cyclin D1flag | / | 56° C. | 1 | minute |
| Cyclin D3 wt Linker 1 | pCDNA3.1cyclinD3Xpress | / | 52° C. | 1 | minute |
| Cyclin D3 wt Linker 2 | pCDNA3.1cyclinD3Xpress | / | 52° C. | 1 | minute |
| Cyclin D3 wt Linker 3 | pCDNA3.1cyclinD3Xpress | / | 52° C. | 1 | minute |
| CDK4 wt Linker 1 | pCDNA6-wtCDK4-HA | / | 56° C. | 1 | minute |
| CDK4 wt Linker 2 | pCDNA6-wtCDK4-HA | / | 54° C. | 1 | minute |
| CDK4 wt Linker 3 | pCDNA6-wtCDK4-HA | / | 54° C. | 1 | minute |
| Fusion Cyclin D1 - | PCR product | PCR product | 56° C. | 2 | minutes |
| Linker 1 - CDK4 wt | Amplification CDK4 wt | Amplification Cyclin | | | |
| | Linker 1 | D1 wt Linker 1 | | | |
| Fusion Cyclin D1 - | PCR product | PCR product | 56° C. | 2 | minutes |
| Linker 2 - CDK4 wt | Amplification CDK4 wt | Amplification Cyclin | | | |
| | Linker 2 | D1 wt Linker 2 | | | |
| Fusion Cyclin D1 - | PCR product | PCR product | 56° C. | 2 | minutes |
| Linker 3 - CDK4 wt | Amplification CDK4 wt | Amplification Cyclin | | | |
| | Linker 3 | D1 wt Linker 3 | | | |
| Fusion Cyclin D3 - | PCR product | PCR product | 56° C. | 2 | minutes |
| Linker 1 - CDK4 wt | Amplification CDK4 wt | Amplification Cyclin | | | |
| | Linker 1 | D3 wt Linker 1 | | | |
| Fusion Cyclin D3 - | PCR product | PCR product | 56° C. | 2 | minutes |
| Linker 2 - CDK4 wt | Amplification CDK4 wt | Amplification Cyclin | | | |
| | Linker 2 | D3 wt Linker 2 | | | |
| Fusion Cyclin D3 - | PCR product | PCR product | 56° C. | 2 | minutes |
| Linker 3 - CDK4 wt | Amplification CDK4 wt | Amplification Cyclin | | | |
| | Linker 3 | D3 wt Linker 3 | | | |

### Example 9

### MCF7 Cells Rendered Quiescent and Stimulated to Grow by Serum and Insulin

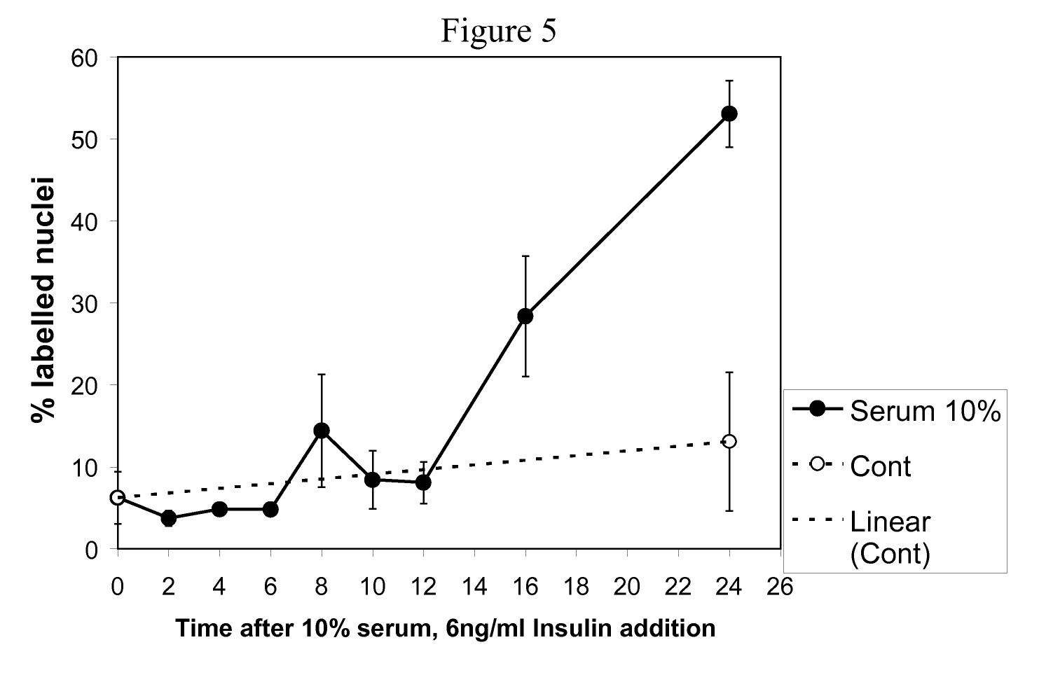

MCF7 cells (ATCC HTB-22) were routinely cultivated in Dulbecco's Modified Eagle Medium (DMEM) comprising 5% serum, 6 ng/ml insulin, 0.1 mM non essential amino acids (NEAA, GIBCO), 1 mM Sodium pyruvate, 50 U/ml Penicillin and 50 μg/ml Streptomycin. The cells were rendered quiescent by incubating them for three days in phenol-free DMEM without serum and insulin, but supplemented with 0.1 mM NEAA (GIBCO), 1 mM Sodium pyruvate, 50 U/ml Penicillin and 50 μg/ml Streptomycin. Cells were kept quiescent (control) or were stimulated to proliferate by adding 5% serum and 6 ng/ml insulin in the culture medium. Samples were harvested 2 h, 4 h, 6 h, 8 h, 10 h, 12 h, 16 h, and 24 h after stimulation of MCF7 cells. Bromodeoxyuridine (BrdU, 10⁻⁴ M) and fluorodeoxyuridine (2.10⁻⁶ M) were added to the cells for the last 24 h of culture. BrdU staining was used to record DNA synthesis. Cells were fixed and the incorporation of BrdU into nuclei was revealed by immunofluorescence as described (M. Baptist, 1995, Exp. Cell Res., 221, 160-171). As shown in FIG. 5, DNA synthesis of quiescent MCF7 was low (full line). DNA synthesis resumed 16 h after stimulation by serum and insulin (FIG. 5, dashed line).

When fulvestrant (10 nM) was added to the culture to block estrogen receptor action during the last 24 h of the incubation without serum and insulin, the proportion of cells starting DNA synthesis dropped to less than 2% (results not shown).

### Example 10

### Induction of MCF7 Cell Proliferation Associated with Time-Dependent Changes in Cyclin Protein Expression Levels and Rb Phosphorylation

MCF7 cells were cultivated and rendered quiescent as described in Example 9. Subsequently, MCF7 cells were kept quiescent or stimulated to proliferate as described in Example 9 for 2 h, 4 h, 6 h, 8 h, 10 h, 12 h, 16 h, 24 h, 28 h, and 32 h. At the indicated times, cells were washed with PBS, scraped in 200 μl of denaturing lysis Laemmli buffer (glycerol 5%, 30 mM Tris-HCl (pH 6.8), 1% SDS, 50 mM DTT, 25 mM NaF, 50 μM vanadate, and protease inhibitors), boiled for 5 min, and frozen. Proteins were resolved by sodium dodecyl sulfate-polyacrylamide gel electrophoresis (SDS-PAGE) and transferred onto polyvinylidene difluoride (PVDF) membranes (Perkin Elmer).

Membranes were probed using the H-22 polyclonal CDK4 antibody (Santa Cruz, Rabbit), the DCS-6 mouse monoclonal anti-cyclin D1 antibody (Neomarkers), the DCS-22 mouse monoclonal anti-cyclin D3 antibody, the E72 anti-cyclin A mouse monoclonal antibody (Neomarkers), the GNS1 anti-cyclin B mouse monoclonal antibody (Neomarkers), and the HE12 anti-cyclin E mouse monoclonal antibody (Thermo Fisher), antibody. Anti-rabbit immunoglobulin antibody or anti-mouse immunoglobulin antibody (GE Healthcare), both coupled to horseradish peroxidase, were used for detection by enhanced chemiluminescence (Western Lightning, Perkin-Elmer, Boston, Mass.). Various exposures of the bands corresponding to these proteins were collected on Hyperfilms (GE Healthcare). Rb phosphorylation is recognized by the upward shift of the apparent molecular weight of the Rb protein detected with the anti-Rb and anti-phospho Rb antibodies used by double immunofluorescent detection using the Odyssey infrared fluorescence scanner (LI-COR). To this end proteins were transferred to low fluorescence PVDF membranes (Millipore) and Rb and its phosphorylated forms were detected using a mixture of the anti-Rb mouse monoclonal antibody (BD Pharmingen) and the rabbit polyclonal T826 phospho-Rb antibody (Thermo Fisher) diluted in the LI-COR blocking buffer. After washings, membranes were incubated with mixed anti-mouse and anti-rabbit secondary antibodies coupled to DyLight 680 and 800 (Perbio Science), respectively.

As shown in FIG. 6, induction of proliferation is associated with a time-dependent increase in the expression of various cyclins. As expected, induction of G1 cyclins such as cyclin D1 and cyclin D3 started early after serum exposure, while the induction of G2 cyclins such as cyclin A and cyclin B was delayed. Phosphorylation of the Rb protein was also stimulated by serum addition. Phosphorylation of the Rb protein started 10 h after stimulation and was maximal 24 h after stimulation.

### Example 11

### Induction of MCF7 Cell Proliferation was Associated with an Increase in Endogeneous CDK4 Phosphorylation on T172

MCF7 cells were cultivated and rendered quiescent as described in Example 9. Subsequently, MCF7 cells were kept quiescent (control) or stimulated to proliferate (serum) for the indicated times as described in Example 9. At the indicated times, cells were washed with PBS, lysed on ice in 1 ml NP-40 lysis buffer. The homogenized (glass/glass) cellular lysate was sonicated 3 times, precleared for 30 minutes with protein A-Sepharose (GE Healthcare, Uppsala, Sweden), and then incubated at 4° C. for 3 h with protein A-Sepharose which had been pre-incubated overnight with 3 μg of monoclonal antibodies against cyclin D1 (DCS-11) or cyclin D3 (DCS-28) (all from Neomarkers, Fremont, Calif.).

Immunoprecipitated proteins were denatured in a buffer comprising 7 M urea, 2 M thiourea, 4% CHAPS, 1% DTE, 1% Pharmalytes 3-10 (GE Healthcare) and 1 mg/ml pefablock before being resolved by 2D gel electrophoresis separations. Proteins were separated by isoelectric focusing using the Protean IEF cell apparatus from Bio-Rad after active in-gel rehydration on immobilized linear pH gradient (pH 3 to 10) strips (GE Healthcare). After loading onto SDS-polyacrylamide slab gels (12.5%) for separation according to molecular mass, proteins were transferred to PVDF membranes (Perkin Elmer). CDK4 was immunodetected using the H-22 polyclonal antibody (Santa Cruz). An anti-mouse immunoglobulin antibody (GE Healthcare) coupled to horseradish peroxidase, was used for detection by enhanced chemiluminescence (Western Lightning, Perkin-Elmer, Boston, Mass.).

The Rb-kinase assay was performed on cyclin D1/CDK4 or cyclin D3/CDK4 complexes immunoprecipitated from cellular extracts of MCF7 quiescent cells treated or not for the indicated time with serum as described previously (Coulonval et al., 2003, Exp. Cell Res., 291, 135-149). After 3 hours of incubation at 4° C. with protein A-Sepharose beads (GE Healthcare), complexes were washed three times with 0.5% NP-40 lysis buffer supplemented with 1 mM DTT and three times with kinase reaction buffer (50 mM Hepes, pH 7.5, 10 mM KCl, 10 mM MgCl2, 2.5 mM EGTA, 1 mM DTT). Washed complexes were resuspended in 40 μl of the kinase reaction buffer containing 2 mM ATP, 0.5 μg of a 48-kDa fragment (amino acid 773-928) of pRb (Sigma), 10 mM β-glycerophosphate, 0.1 mM orthovanadate, 1 mM NaF, 60 μg/ml pefabloc, and 1 μg/ml leupeptine and incubated for 30 min at 30° C. with gentle agitation. Reactions were stopped by adding 60 μl of twice-concentrated Laemmli buffer (glycerol 10%, 60 mM Tris-HCl (pH 6.8), 2% SDS, 100 mM DTT, 50 mM NaF, 100 μM vanadate, and protease inhibitors) and boiling for 5 min. Proteins were resolved by SDS-PAGE and transferred on PVDF membranes (Perkin Elmer) before the phosphorylation on T826 of the pRb fragment was detected using the phosphor-specific pRb (T826) antibody (Thermo Fisher). Membranes were then reprobed using the H-22 polyclonal CDK4 antibody (Santa Cruz, Rabbit). An anti-rabbit or anti-mouse immunoglobulin antibody (GE Healthcare) coupled to horseradish peroxidase was used for detection by enhanced chemiluminescence (Western Lightning; Perkin-Elmer, Boston, Mass.).

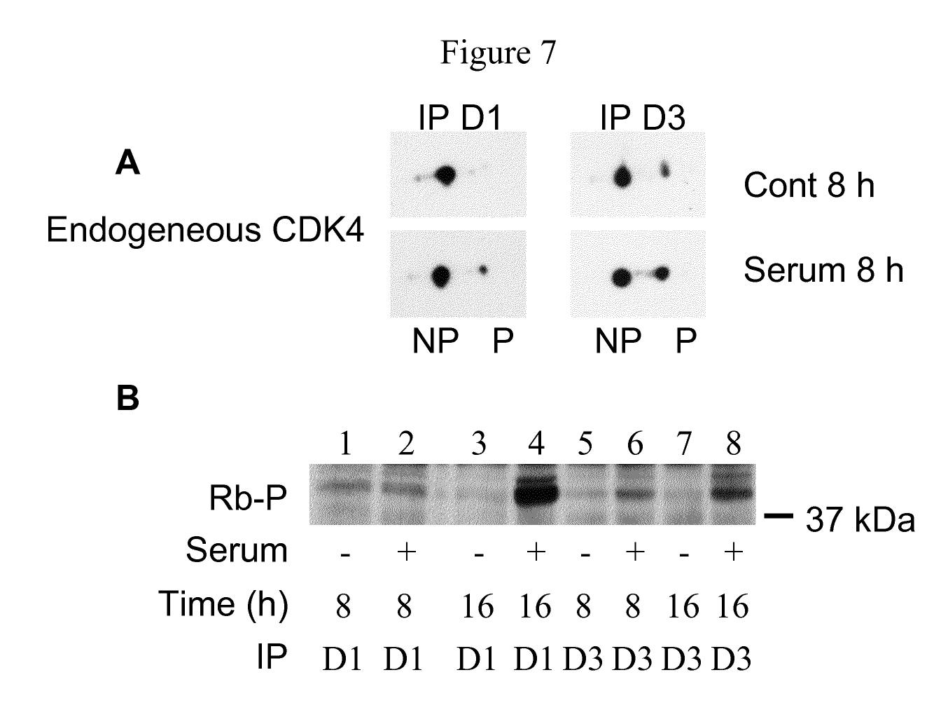

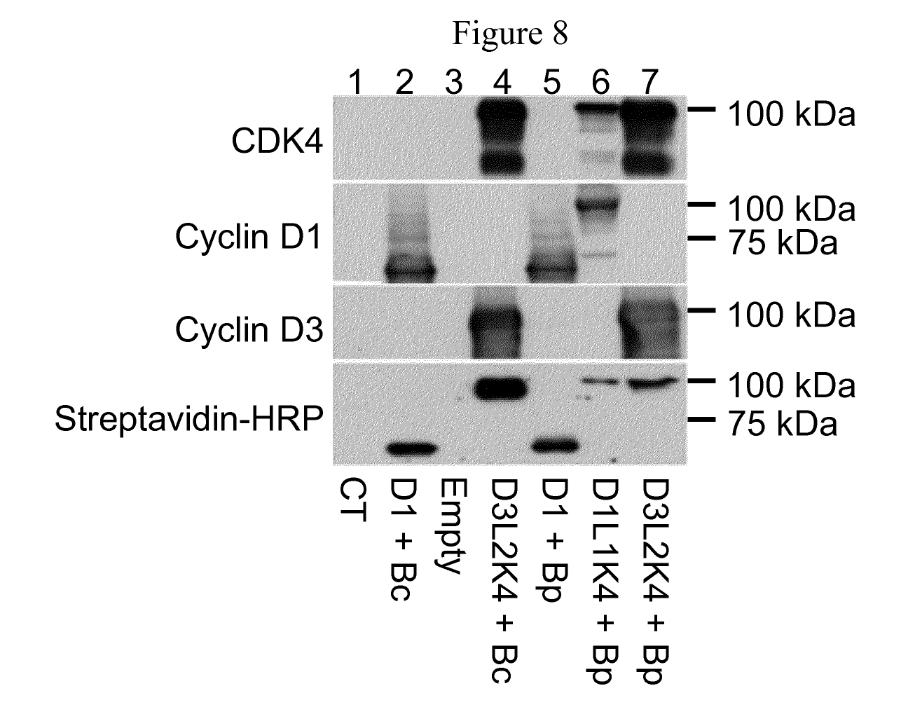

As shown in FIG. 7A, endogenous CDK4 present in both cyclin D1/CDK4 and cyclin D3/CDK4 complexes was phosphorylated upon exposure to serum. Moreover, as shown in FIG. 7B, cellular extracts from MCF7 cells stimulated for 8 h or for 16 h show Rb kinase activity, while those of non-stimulated cells do not. Hence, the MCF7 cell line is a suitable model to trace the activation of CDK4 by its phosphorylation on T172 upon induction of proliferation.

### Example 12

### Transgene Expression from the Constructed Lentiviral Vectors Upon Transient Transfection in HEK293T Cells

In order to verify if transgene expression, in particular expression of the cyclin D1/CDK4 fusion protein or cyclin D3/CDK4 fusion protein, could be driven by the lentiviral constructions described in Example 8, HEK293T cells (ATCC CRL 11268) were transiently transfected with the corresponding DNA produced with the Qiagen midiprep kit following the instructions of the manufacturer. HEK293T cells were cultivated in DMEM medium (Invitrogen) supplemented with pyruvate (1 mM), penicillin (50 U/ml), Streptomycin (50 μg/ml) and 10% foetal calf serum. 6 10⁵ cells were seeded in 6-well plates the day before the transfection by the calcium phosphate method.

On the day of transfection, plasmids (1 μg each) were diluted in 200 μl Tris buffer (Tris 50 mM; EDTA 1 mM) supplemented with 50 μl of 2.5M CaCl₂. This dilution was added dropwise on 250 μl HBS buffer (HEPES 50 mM (pH 7.05); KCl 10 mM; dextrose 12 mM; NaCl 280 mM; Na₂HPO₄ 1.5 mM). After 10 min incubation at 37° C., 250 μl of the mixture was added on the cells. DNA was left on the cells for 6 hours. Thereafter, medium was refreshed and cells were incubated for 48 h in HEK293T cell culture medium supplemented with 0.1 mM biotin. Then, cells were lysed as described in Example 10. Proteins were resolved by SDS-PAGE and transferred onto PVDF membranes (Perkin Elmer). Membranes were then probed using the H-22 polyclonal CDK4 antibody (Santa Cruz, Rabbit), the DCS-6 monoclonal cyclin D1 (Neomarkers, mouse), the DCS-22 monoclonal cyclin D3 antibody (Neomarkers, mouse), and Streptavidin coupled to horseradish peroxidase (GE Healthcare). Anti-rabbit immunoglobulin antibody or anti-mouse immunoglobulin antibody (GE Healthcare), both coupled to horseradish peroxidase, were used for detection by enhanced chemiluminescence (Western Lightning, Perkin-Elmer, Boston, Mass.). Various exposures of the bands corresponding to these proteins were collected on Hyperfilms (GE Healthcare).

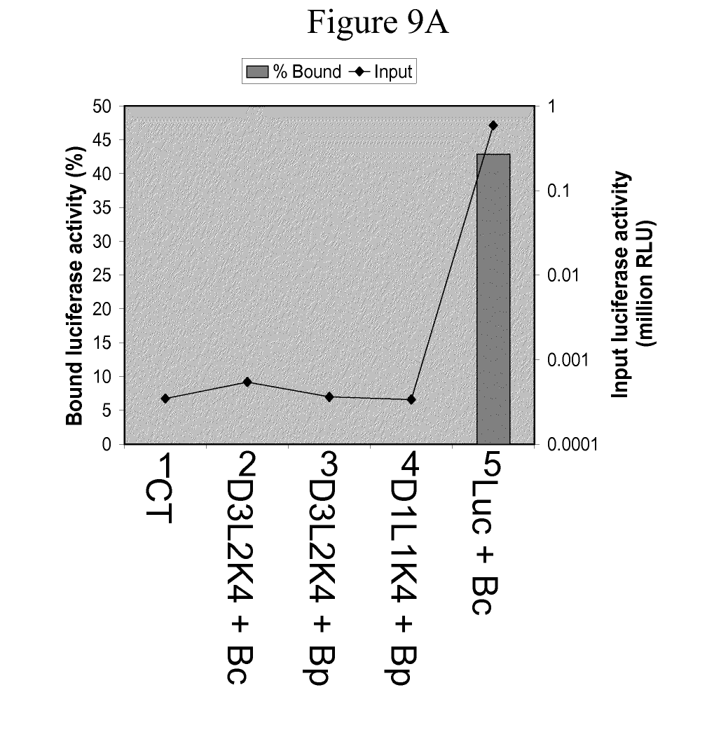

As shown in FIG. 8, all transgenes were expressed in HEK293T cells. As all tested constructs contained either a BirA/mCherry or a BirA/puromycin resistance gene fusion following via an IRES sequence the expression module of the Avi-tagged transgene, biotinylation of the trangenes was expected. As indicated in the lowest immunoblot in FIG. 8, this was indeed the case.

### Example 13

### Immobilization on Streptavin-Coated Sepharose Beads of Transgenes Expressed after Transient Transfection in HEK293T Cells and Assay for their Activity

In order to verify if the transgenes expressed in HEK293T cells after transient transfection could be immobilized on streptavidin-coated sepharose bead and were enzymatically active, HEK293T cells were cultivated and transfected with the indicated plasmids as described in Example 12. The inert plasmid pBluescript was used as negative transfection control (noted CT) in the experiments shown in FIG. 8.

After transfection, cells were washed with PBS and lysed on ice in 1 ml NP-40 lysis buffer as described in Example 11. The homogenized (glass/glass) cellular lysate was sonicated 3 times and stored at −20° C. Equal amount of protein from the defrozen lysates were then incubated for 3 h at 4° C. under slight shaking with 5 μl of streptavidin-coated sepharose beads (GE Healthcare) which had been prewashed three times with NP-40 lysis buffer without inhibitors. After 3 h, beads bound with transgene are washed three times with cold NP-40 lysis buffer without inhibitors before enzymatic activities are measured.

For the luciferase assay, cellular extracts were diluted to a final volume of 200 μl. Input luciferase activity was assessed in two 20 μl samples of the bead suspension after the 3 hour incubation. Two 20 μl samples of the bead supernatant recovered after the first centrifugation of the beads were spotted in a black 96 well plate for luciferase assay (unbound luciferase activity). After 3 washes with cold NP-40 lysis buffer without inhibitors, beads bound with luciferase were decanted by centrifugation and resuspended in 160 μl NP-40 lysis buffer without inhibitors. Two 20 μl samples of the resuspended washed bead (bound luciferase activity) were spotted in a black 96 well plate for luciferase assay. Luciferase assay was started by adding 20 μl of luciferase reaction buffer (Tricine 40 mM; (MgCO₃)₄Mg(OH)₂.5H₂O 2.14 mM; MgSO₄.7H₂O 5.34 mM; DTT 66.6 mM; EDTA 0.2 mM; coenzyme A (40 mg/50 ml); ATP 0.7 mM; luciferine 940 μM) and recording the emitted light for 1 sec in a Berthold luminometer. Bound luciferase activity was expressed relative to the total luciferase activity, i.e., bound and unbound activities.

The pRb kinase assay was performed on the cellular extract bound to the streptavidin-coated sepharose beads as described previously (Coulonval et al., 2003, Exp. Cell Res., 291, 135-149). After 3 hours of incubation at 4° C. with streptavidin-coated sepharose beads, complexes were washed three times with 0.5% NP-40 lysis buffer supplemented with 1 mM DTT and three times with kinase reaction buffer (50 mM Hepes, pH 7.5, 10 mM KCl, 10 mM MgCl2, 2.5 mM EGTA, 1 mM DTT). Washed complexes were resuspended in 40 μl of the kinase reaction buffer containing 2 mM ATP, 0.5 μg of a 48-kDa fragment (amino acid 773-928) of pRb (Sigma), 10 mM β-glycerophosphate, 0.1 mM orthovanadate, 1 mM NaF, 60 μg/ml pefabloc, and 1 μg/ml leupeptine and incubated for 30 min at 30° C. with gentle agitation. Reactions were stopped by adding 60 μl of twice-concentrated Laemmli buffer (glycerol 10%, 60 mM Tris-HCl (pH 6.8), 2% SDS, 100 mM DTT, 50 mM NaF, 100 μM vanadate, and protease inhibitors) and boiling for 5 min. Proteins were resolved by SDS-PAGE and transferred on PVDF membranes (Perkin Elmer) before the phosphorylation on T826 of the pRb fragment was detected using the phosphor-specific pRb (T826) antibody (Thermo Fisher). Membranes were then reprobed using the H-22 polyclonal CDK4 antibody (Santa Cruz, Rabbit). An anti-rabbit or anti-mouse immunoglobulin antibody (GE Healthcare) coupled to horseradish peroxidase was used for detection by enhanced chemiluminescence (Western Lightning; Perkin-Elmer, Boston, Mass.).

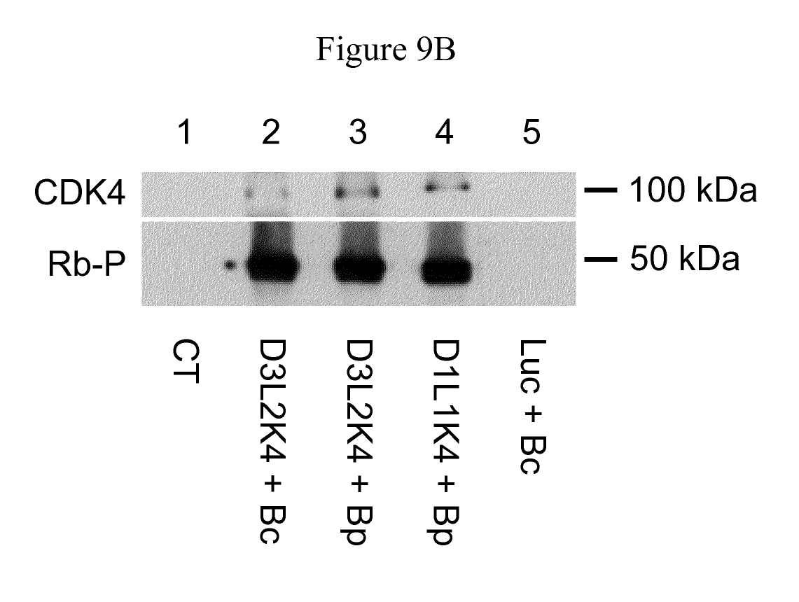

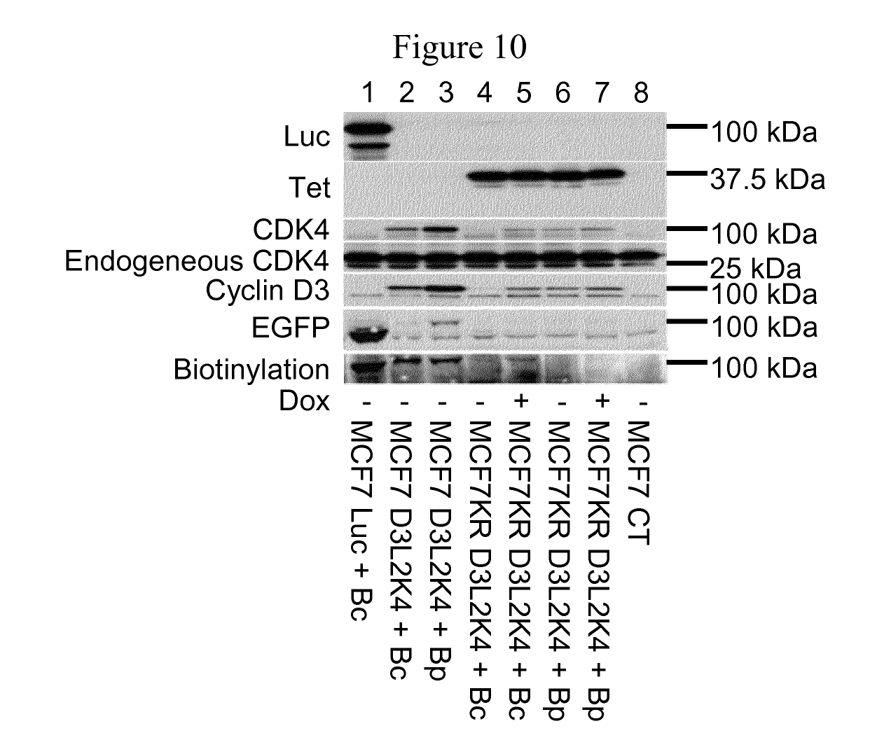

FIG. 9A shows that up to 40% of the total luciferase activity was bound to the streptavidin-coated sepharose beads in cellular extract of HEK293T cells transfected with an expression vector of an Avi-tagged luciferase. No luciferase activity was detected when the cells were transfected with vector allowing the expression of a cyclinD/CDK4 fusion protein. By contrast, Rb kinase activity was only bound to beads incubated with extracts of HEK293T cells transfected with vectors allowing the expression of a cyclinD/CDK4 fusion protein. Both cyclin D1/CDK4 and cyclin D3/CDK4 fusion proteins were active (FIG. 9B). Moreover, the Rb kinase activity could be recovered both when a BirA/mCherry or a BirA/puromycin resistance gene fusion was expressed (FIG. 9B). Taken together, these results demonstrate that the transgenes were specifically immobilized on streptavidin-coated beads, i.e., they were correctly biotinylated, and that they remained enzymatically active.

### Example 14

### Transgenes were Expressed and Biotinylated in Stable Cell Populations of MCF7 Cells Transduced with Lentiviruses Allowing the Expression of Avi-Tagged Transgenes and a BirA/mCherry Fusion Protein

Lentiviruses were produced by co-transfecting HEK293T cells with 3 μg of the lentiviral vectors constructed as described in Example 8 and 1.5 and 3 μg of the packaging vectors pMD2.G and pCMVΔR8.2, respectively. pMD2.G (Addgene plasmid #12259) and pCMVΔR8.2 (Addgene plasmid #12263) plasmids were described previously by Pr. Trono's team (University of Lausanne, Switzerland) (Wiznerovicz et al., supra). To this end, these plasmids were first precipitated together with sodium acetate and resuspended in 10 μl of ultrapure DNAse-free water. The DNA mixture was added to 190 μl of Tris buffer (Tris 50 mM; EDTA 1 mM) and 50 μl of 2.5M CaCl₂. This solution was added dropwise on 250 μl HBS buffer (HEPES 50 mM (pH 7.05); KCl 10 mM; dextrose 12 mM; NaCl 280 mM; Na₂HPO₄ 1.5 mM). After incubation for 10 min at 37° C., 250 μl of the mixture was added on 6.10⁵ HEK293T cells seeded in two wells of a 6-well plate per construct, the day before transfection (2.5 ml of medium/well). Chloroquine (25 μM) was added in each well before incubation at 36° C. for 6 hours. Thereafter, the medium was refreshed and cells were incubated at 36° C. in 2.5 ml HEK293T cell culture medium per well. After 48 hours, the virus-containing medium was harvested and filtered through a 0.45-mm low protein-binding filter (Millipore, Billerica, Mass., USA). Aliquots were stored at −70° C. Transduction of MCF7 cells or MCF7/KR cells done in triplicate, was performed by mixing 10⁴ cells with 200 μl viral supernatant in a 96-well plate. MCF7/KR cells are MCF7 cells expressing the Tet/KRAB repressor after transduction with the pLVTH/KR plasmid (Addgene plasmid #12249) described by the team of Pr. Trono (University of Lausanne, Switzerland) (Wiznerovicz et al., supra).

After 72 h, the cells were trypsinized and replicates were pooled in a 24-well plate with fresh MCF7 culture medium. Cell populations were amplified before characterization. Transgene expression was assessed by western blotting as described in Example 2. Membranes were probed using the H-22 polyclonal CDK4 antibody (Santa Cruz, Rabbit), the anti-TetR mouse monoclonal antibody (Clontech), the anti-Luc mouse monoclonal antibody (Santa Cruz), or the DCS-22 monoclonal cyclin D3 antibody (Neomarkers). Anti-rabbit immunoglobulin antibody (GE Healthcare) or anti-mouse immunoglobulin antibody (GE Healthcare), both coupled to horseradish peroxidase were used for detection by enhanced chemiluminescence (Western Lightning, Perkin-Elmer, Boston, Mass.).

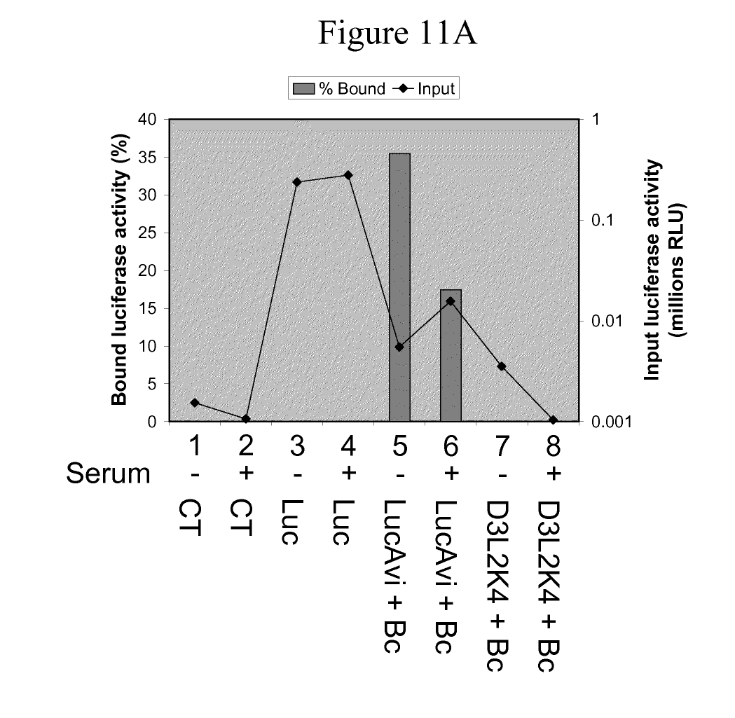

As shown in FIG. 10, stable MCF7 cell populations created by infection with lentiviruses produced with the vectors described in Example 8 expressed in a constitutive way luciferase or the Cyclin D3/CDK4 fusion protein. Basal expression of the Cyclin D3/CDK4 fusion was low to undetectable in the MCF7/KR cells. These cells express the Tet/KRAB repressor which binds to the tet response element present in front of the transgene expression module in the vectors described in Example 8. By binding to this tet response element, the Tet/KRAB repressor epigenetically represses the transcription of elements located 3 kb upstream or downstream of the tet response element. This repression is relieved by addition of doxycycline which inhibits binding to DNA of the Tet domain of the Tet/KRAB repressor.

As shown in FIG. 10, addition of doxycycline to the MCF7/KR cells transduced with the lentiviral vectors allowing the expression of the Cyclin D3/CDK4 fusion enhanced the expression of the latter. These results indicate that the lentiviral vectors described in Example 8 were functional and allowed the generation of a cell line with constitutive or inducible expression of the transgenes. Moreover, FIG. 10 also shows that the transgenes can be biotinylated especially in the cell populations with constitutive expression of the transgenes.

### Example 15

### Purified Immobilized Biotinylated Avi-Tagged Cyclin D3/CDK4 Fusion Expressed in Stable Cell Populations of MCF7 Cells Displayed a Rb-Kinase Activity Modulated According to their Proliferation Status

In order to verify if transgenes expressed in MCF7 cells after transduction with the vectors described in Example 8 could be immobilized on streptavidin-coated sepharose bead and were enzymatically active, the MCF7 cells expressing the luciferase or the cyclin D3/CDK4 fusion protein described in the Example 14 were cultivated in 10 cm Petri dishes with 10 ml DMEM medium supplemented with 5% serum, insulin 6 ng/ml, NEAA (Gibco) 0.1 mM, Sodium pyruvate (1 mM), Penicillin (50 U/ml) and Streptomycin (50 μg/ml). The cells were rendered quiescent by incubating them for three days in phenol-free DMEM without serum and insulin, but supplemented with biotin (0.1 mM), NEAA (0.1 mM, Gibco), Sodium pyruvate (1 mM), Penicillin (50 U/ml) and Streptomycin (50 μg/ml). During the last 24 hours, fulvestrant (10 nM) was added to the culture to block estrogen receptor action. Cells were stimulated to proliferate by adding 5% serum, 6 ng/ml insulin and 100 nM β-estradiol in the culture medium for 16 hours in the continuous presence of biotin (0.1 mM). At the end of the culture, cells were washed with PBS and lysed on ice in 1 ml NP-40 lysis buffer as described in Example 10. The homogenized (glass/glass) cellular lysate was sonicated 3 times and stored at −20° C. Equal amounts of protein from the defrozen lysate were then incubated for 3 h at 4° C. under slight shaking with 5 μl of streptavidin-coated sepharose beads which had been prewashed three times with NP-40 lysis buffer without inhibitors. After 3 h, beads bound with transgene were washed three times with cold NP-40 lysis buffer without inhibitors before enzymatic activities were measured.

For the luciferase assay, cellular extracts were diluted to a final volume of 200 μl. Input luciferase activity was assessed in two 20 μl samples of the bead suspension after the 3 hour incubation. Two 20 μl samples of the bead supernatant recovered after the first centrifugation of the beads were spotted in a black 96 well plate for luciferase assay (unbound luciferase activity). After 3 washes with cold NP-40 lysis buffer without inhibitors, beads bound with luciferase were decanted by centrifugation and resuspended in 160 μl NP-40 lysis buffer without inhibitors. Two 20 μl samples of the resuspended washed beads (bound luciferase activity) were spotted in a black 96 well plate for luciferase assay. Luciferase assay was started by adding 20 μl of luciferase reaction buffer (Tricine 40 mM; (MgCO₃)₄Mg(OH)₂.5H₂O 2.14 mM; MgSO₄.7H₂O 5.34 mM; DTT 66.6 mM; EDTA 0.2 mM; coenzyme A (40 mg/50 ml); ATP 0.7 mM; luciferine 940 μM) and recording the emitted light for 1 sec in a Berthold luminometer. Bound luciferase activity was expressed relative to the total luciferase activity, i.e., bound and unbound activities.

The pRb kinase assay was performed on the cellular extract bound to the streptavidin-coated sepharose beads as described previously (Coulonval et al., supra). After 3 hours of incubation at 4° C. with streptavidin-coated sepharose beads, complexes were washed three times with 0.5% NP-40 lysis buffer supplemented with 1 mM DTT and three times with the kinase reaction buffer as described in Example 13. Washed complexes were resuspended in 40 μl of the kinase reaction buffer containing 2 mM ATP, 0.5 μg of a 48-kDa fragment (aa 773-928) of pRb (Sigma), 10 mM β-glycerophosphate, 0.1 mM orthovanadate, 1 mM NaF, 60 μg/ml pefabloc, and 1 μg/ml leupeptine and incubated for 30 min at 30° C. with occasional gentle agitation. Reactions were stopped by adding 60 μl of twice-concentrated Laemmli buffer (glycerol 10%, 60 mM Tris-HCl (pH 6.8), 2% SDS, 100 mM DTT, 50 mM NaF, 100 μM vanadate, and protease inhibitors) and boiling for 5 min. Proteins were resolved by SDS-PAGE and transferred on PVDF membranes (Perkin Elmer) before the phosphorylation on T826 of the pRb fragment was detected using the phospho-specific anti-pRb (T826) antibody (Thermo Fisher). Membranes were then reprobed using the H-22 polyclonal CDK4 antibody (Santa Cruz, Rabbit). An anti-rabbit or anti-mouse immunoglobulin antibody (GE Healthcare) coupled to horseradish peroxidase, was used for detection by enhanced chemiluminescence (Western Lightning; Perkin-Elmer, Boston, Mass.).

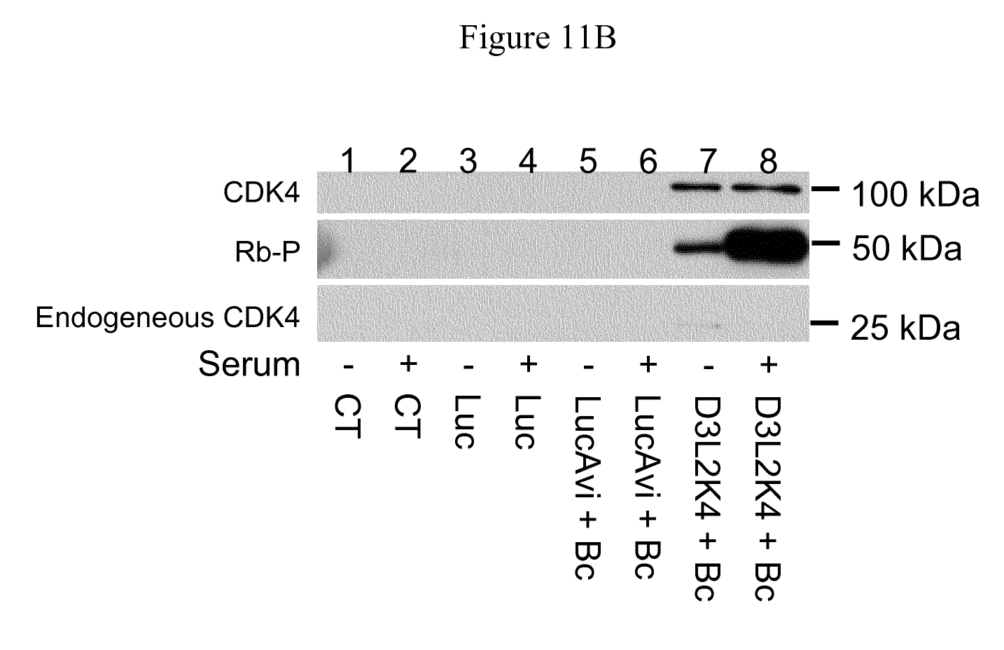

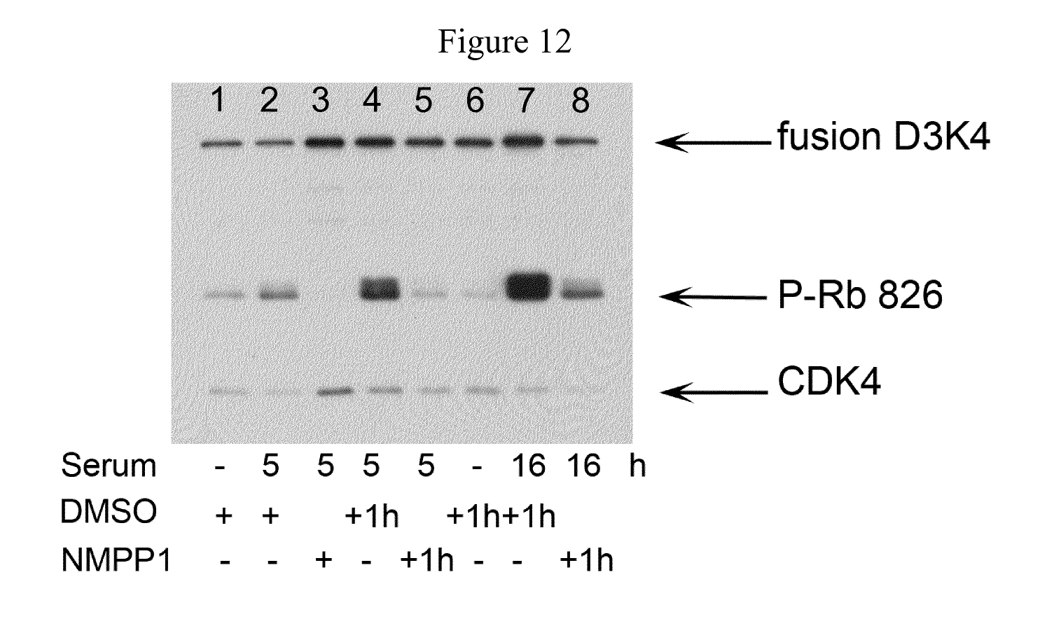

As shown in FIG. 11A, luciferase activity was detected on the streptavidin-coated beads incubated with extracts of MCF7 cells expressing a biotinylable luciferase together with the BirA/mCherry fusion (noted LucAvi+Bc). This was not the case when the cells expressed the luciferase lacking the Avi-tag (noted Luc) or the Cyclin D3/CDK4 fusion together with the BirA/mCherry fusion (D3L2K4+Bc). On the other hand, neither the streptavidin-coated beads incubated with extract from MCF7 expressing the Avi-tagged luciferase nor those incubated with extract from MCF7 expressing the untagged luciferase displayed any Rb kinase activity (FIG. 11B, lanes 3 to 6)). Rb kinase activity was detectable in quiescent MCF7 cells expressing a biotinylable Cyclin D3/CDK4 fusion protein (FIG. 11B, lane 7). The Rb kinase activity was strongly enhanced 16 h after serum/insulin/β-estradion stimulation of the MCF7 cells (FIG. 11B, lane 8).

These results show that biotinylated transgenes expressed in MCF7 after transduction with the vectors described in Example 8 could be purified with streptavidin-coated sepharose beads and remained enzymatically active. Furthermore, only beads incubated with extracts from MCF7 cells expressing a biotinylable Cyclin D3/CDK4 fusion possessed detectable Rb kinase activity. Finally, this activity which was heavily regulated in response to serum/insulin stimulation perfectly mimicked the activity of the endogenous CDK4 described in Example 10. Biotinylated Avi-tagged Cyclin D3/CDK4 fusion protein expressed in a stable cell population of MCF7 cells after transduction with the vectors described in Example 8 is thus a perfect reporter molecule of the activation status of endogenous CDK4 described in Example 11.

Example 15 thus brings the proof of concept that a reporter molecule comprising cyclin D3/CDK4 fusion protein coupled to an Avi-tag and expressed in MCF7 together with BirA/mCherry or BirA/puro fusion can be immobilized on streptavidin-coated supports and conserves an enzymatic Rb kinase activity which is modulated according to the proliferation status of the cells. Since the Rb kinase activity of the cyclin D3/CDK4 fusion can be specifically and efficiently followed on immobilized streptavidin, this reporter offers the possibility to set up a miniaturized high throughput screening assay to identify molecules or genes for instance by their silencing with siRNA, affecting the activating phosphorylation of CDK4, more particularly on T172.

### Example 16

### Purified Immobilized Biotinylated Avi-Tagged Cyclin D3/CDK4 Fusion Expressed in Stable Cell Populations of HCT116K7AS Cells Displays a Rb-Kinase Activity Modulated According to their Proliferation Status

In order to verify if transgenes expressed in HCT116K7AS cells after transduction with the vectors described in Example 8 could be immobilized on streptavidin-coated sepharose beads and were enzymatically active, the HCT116K7AS cells expressing the cyclin D3/CDK4 fusion protein described in the Example 14 were cultivated in 6 cm Petri dishes with 10 ml DMEM medium supplemented with 10% serum, Sodium pyruvate (1 mM), Penicillin (50 U/ml) and Streptomycin (50 μg/ml). These cells express only a mutated version of CDK7 (CDK7AS) inhibitable by bulky ATP analogs such as 1-NMPP1 (Santa Cruz). The cells were rendered quiescent by incubating them for three days in DMEM without serum, but supplemented with biotin (0.1 mM), Sodium pyruvate (1 mM), Penicillin (50 U/ml) and Streptomycin (50 μg/ml). Cells were stimulated to proliferate by adding 10% serum for 5 or 16 hours in the continuous presence of biotin (0.1 mM). As indicated, vehicle (DMSO) or CDK7AS inhibitor (1-NMPP1) were added together with serum for the same time or for an additional pulse of 1 hour. At the end of the culture, cells were washed with PBS and lysed on ice in 1 ml NP-40 lysis buffer as described in Example 10. The homogenized (glass/glass) cellular lysate was sonicated 3 times and stored at −70° C. Equal amounts of protein (corresponding to one third of the total lysate volume) from the defrozen lysate were then incubated for 3 h at 4° C. under slight shaking with 20 μl of streptavidin-coated sepharose beads (GE Healthcare) which had been prewashed three times with NP-40 lysis buffer without inhibitors. After 3 h, beads bound with transgenes were washed three times with cold NP-40 lysis buffer supplemented with 1 mM DTT without inhibitors.

The pRb kinase assay was performed on the cellular extract bound to the streptavidin-coated sepharose beads as described previously (Coulonval et al., supra). After 3 hours of incubation at 4° C. with streptavidin-coated sepharose beads, complexes were washed three times with 0.5% NP-40 lysis buffer supplemented with 1 mM DTT and three times with the kinase reaction buffer (50 mM Hepes, pH 7.5, 10 mM KCl, 10 mM MgCl2, 2.5 mM EGTA, 1 mM DTT). Washed complexes were resuspended in 40 μl of the kinase reaction buffer containing 2 mM ATP, 0.3 μg of a 48-kDa fragment (aa 773-928) of pRb (Sigma), 10 mM β-glycerophosphate, 0.1 mM orthovanadate, 1 mM NaF, 60 μg/ml pefabloc, and 1 μg/ml leupeptine and incubated for 30 min at 30° C. with continuous gentle agitation. Reactions were stopped by adding 60 μl of twice-concentrated Laemmli buffer (glycerol 10%, 60 mM Tris-HCl (pH 6.8), 2% SDS, 100 mM DTT, 50 mM NaF, 100 μM vanadate, and protease inhibitors) and boiling for 5 min. Proteins were resolved by SDS-PAGE and transferred on PVDF membranes (Perkin Elmer). The phosphorylation on T826 of the pRb fragment was detected using a phospho-specific anti-pRb (T826) antibody (Epitomics). Membranes were then reprobed using the H-22 rabbit polyclonal CDK4 antibody (Santa Cruz). An anti-rabbit immunoglobulin antibody (GE Healthcare) coupled to horseradish peroxidase, was used for detection by enhanced chemiluminescence (Western Lightning; Perkin-Elmer, Boston, Mass.).

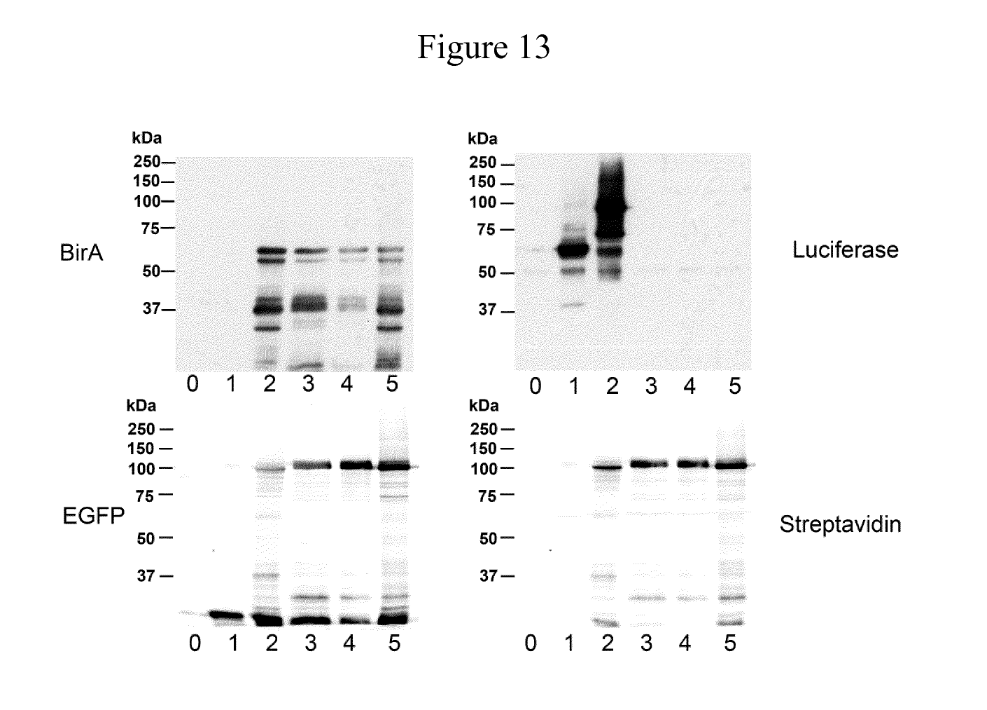

As shown in FIG. 12, Rb kinase activity bound on streptavidin sepharose beads reporting the activity of the exogeneous biotinylatable Cyclin D3/CDK4 fusion expressed in HCT116K7AS cells was induced by serum as early as after 5 hours (lane 2). This induction is prevented when the CDK7AS inhibitor is present together with serum for 5 hours (lane 3). A similar inhibition by the CDK7AS inhibitor is observed when the latter is added during the last hour of a 6 hours serum stimulation period (compare lane 4 to lane 5). Maximal Rb kinase activity is detectable in HCT116K7AS cells expressing a biotinylatable Cyclin D3/CDK4 fusion protein after 17 hours of stimulation (lane 6). This stimulation is severely impaired when the CDK7AS inhibitor 1-NMPP1 is added dung the last hour of serum stimulation (lane 7).

Example 16 thus shows that biotinylated Cyclin D3/CDK4 fusion protein expressed in HCT116K7AS after transduction with the vectors described in Example 8 could be purified with streptavidin-coated sepharose beads and remained enzymatically active. Finally, this activity which was heavily regulated in response to serum stimulation and inhibitable by the CDK7AS inhibitor 1-NMPP1 perfectly mimicked the activity described in Example 15 of the biotinylated Avi-tagged Cyclin D3/CDK4 fusion protein expressed in a stable cell population of MCF7 cells after transduction with the vectors described in Example 8. Furthermore, the changes in Rb kinase activity of extracts from HCT116K7AS cells expressing a biotinylatable Cyclin D3/CDK4 fusion after incubation with serum together or not with a CDK7AS inhibitor perfectly mimicks the changes in activity of the endogeneous CDK4 of the wild type HCT116K7AS cells treated in the same way previously described (Bisteau et al., PLoS Genet. 2013 May; 9(5):e1003546).

Example 16 thus brings the proof of concept that a reporter molecule comprising cyclin D3/CDK4 fusion protein coupled to an Avi-tag and expressed together with BirA/mCherry fusion can be immobilized on streptavidin-coated matrices and conserves an enzymatic Rb kinase activity which is modulated according to the proliferation status of the cells. This occurs in different mammalian cells. Since the Rb kinase activity of the cyclin D3/CDK4 fusion can be specifically and efficiently followed on immobilized streptavidin, this reporter offers the possibility to set up a miniaturized high throughput screening assay to identify molecules or genes for instance by their silencing with siRNAs, affecting the activating phosphorylation of CDK4, more particularly on T172. Furthermore, inhibition of the cyclin D3/CDK4 fusion protein coupled to an Avi-tag by a CDK7AS inhibitor in HCT116K7AS cells provides an easy internal reference to which the effects of SiRNAs or other small molecules can be advantageously compared.

### Example 17

### Mutated and Wild-Type Biotinylatable Prescission-Cleavable CCND3/CDK4 Reporter Expression from the Constructed Lentiviral Vectors Upon Transient Transfection in HEK293T Cells

In order to verify if biotinylatable or non-biotinylatable cyclin D3/CDK4/EGFP or luciferase/EGFP fusion proteins separated from the Avi-tagged EGFP part of the fusion by a linker cleavable by the Prescission protease could be driven by the lentiviral constructions described in Example 8, HEK293T cells (ATCC CRL 11268) were transiently transfected with the corresponding DNA produced with the Qiagen midiprep kit following the instructions of the manufacturer. HEK293T cells were cultivated in DMEM medium (Invitrogen) supplemented with pyruvate (1 mM), penicillin (50 U/ml), Streptomycin (50 μg/ml) and 10% foetal calf serum. 6 10⁵ cells were seeded in 6-well plates the day before the transfection by the calcium phosphate method.

On the day of transfection, plasmids (1 μg each) were diluted in 200 μl Tris buffer (Tris 50 mM; EDTA 1 mM) supplemented with 50 μl of 2.5M CaCl₂. This dilution was added dropwise on 250 μl HBS buffer (HEPES 50 mM (pH 7.05); KCl 10 mM; dextrose 12 mM; NaCl 280 mM; Na₂HPO₄ 1.5 mM). After 10 min incubation at 37° C., 250 μl of the mixture was added on the cells. DNA was left on the cells for 6 hours. Thereafter, medium was refreshed and cells were incubated for 48 h in HEK293T cell culture medium supplemented with 0.1 mM biotin. Then, cells were lysed with the following buffer (100 mM NaCl, 50 mM Tris-HCl pH 7.5, 0.1% SDS, 1% Na deoxycholate, EDTA 1 mM, EGTA 1 mM, 50 mM NaF, 1 mM Orthovanadate, 1 mM β-glycerophosphate, 1 μg/ml leupeptin, 60 μg/ml Pefabloc and 10% glycerol). Proteins were resolved by SDS-PAGE and transferred onto PVDF membranes (Perkin Elmer) or low fluorescence PVDF membranes (Millipore) for chemiluminescence or Odyssey detection, respectively. Membranes were then probed using the anti-BirA chicken antibody (Sigma) and the mouse anti-luciferase antibody (Santa Cruz). Anti-chicken immunoglobulin (Santa-Cruz) or Anti-rabbit immunoglobulin antibody (GE Healthcare), both coupled to horseradish peroxidase, were used for detection by enhanced chemiluminescence (Western Lightning, Perkin-Elmer, Boston, Mass.). Various exposures of the bands corresponding to these proteins were collected on Hyperfilms (GE Healthcare). Mouse anti-GFP monoclonal antibody (Santa Cruz) was detected with anti-mouse immunoglobulin antibody (Perbio) coupled to Dylight800 with the Odyssey system (Western Lightning; Perkin-Elmer, Boston, Mass.). Biotinylated proteins were detected in parallel with streptavidin coupled to AlexaFluor680 (InVitrogen) using the Odyssey system.

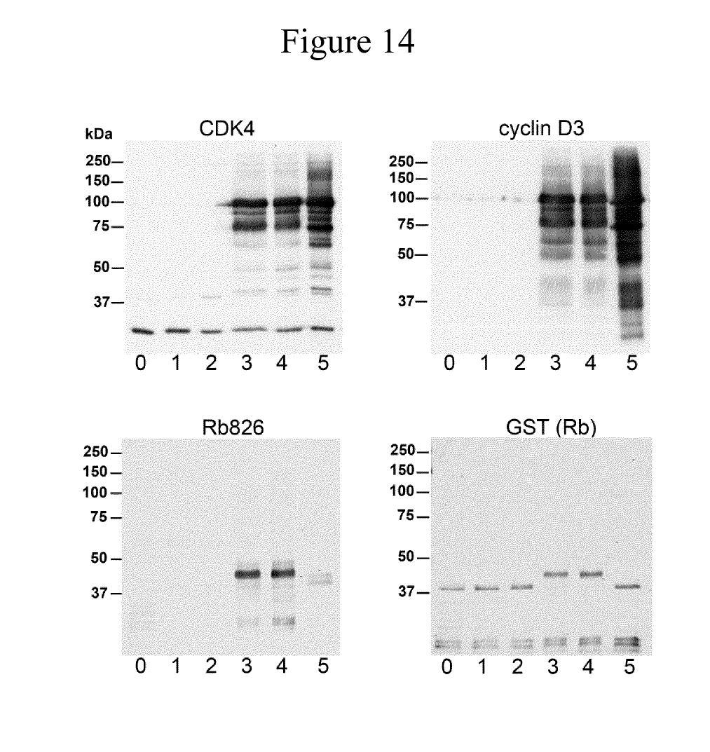

As shown in FIG. 13, all transgenes were expressed in HEK293T cells. As all tested constructs contained a BirA/mCherry fusion following via an IRES sequence the expression module of the Avi-tagged transgenes, biotinylation of the trangenes was expected. The panel noted BirA confirms the expression of the BirA/mCherry fusion protein. Expression of luciferase was observed only in HEK293T cells transfected with the pdLVCTEGFPtetOV5HisLuc and pWFAviIBC_Luc constructs. EGFP-Fusion proteins were detected in all cell extracts of HEK293T except the extracts from control cells and of cells transfected with the pdLVCTEGFPtetOV5HisLuc construct. All these fusion proteins are biotinylated as revealed by detection with AlexaFluor680-labelled streptavidin.

### Example 18

### Both Wild-Type Prescission-Cleavable and Uncleavable Biotinylatable CCND3/CDK4 Reporters Expressed from the Constructed Lentiviral Vectors Upon Transient Transfection in HEK293T Cells Display Rb-Kinase Activity

In order to verify if the biotinylatable cyclin D3/CDK4 fusion protein with a linker cleavable by the Prescission protease driven by the lentiviral constructions described in Example 8 also possess Rb-kinase activity, HEK293T cells (ATCC CRL 11268) were transiently transfected with the corresponding DNA produced with the Qiagen midiprep kit following the instructions of the manufacturer. HEK293T cells were cultivated in DMEM medium (Invitrogen) supplemented with pyruvate (1 mM), penicillin (50 U/ml), Streptomycin (50 μg/ml) and 10% foetal calf serum. 610⁵ cells were seeded in 6-well plates the day before the transfection by the calcium phosphate method.

On the day of transfection, plasmids (1 μg each) were diluted in 200 μl Tris buffer (Tris 50 mM; EDTA 1 mM) supplemented with 50 μl of 2.5M CaCl₂. This dilution was added dropwise on 250 μl HBS buffer (HEPES 50 mM (pH 7.05); KCl 10 mM; dextrose 12 mM; NaCl 280 mM; Na₂HPO₄ 1.5 mM). After 10 min incubation at 37° C., 250 μl of the mixture was added on the cells. DNA was left on the cells for 6 hours. Thereafter, medium was refreshed and cells were incubated for 48 h in HEK293T cell culture medium supplemented with 0.1 mM biotin. Then, cells were lysed as described in Example 10. Proteins were resolved by SDS-PAGE and transferred onto PVDF membranes (Perkin Elmer). Membranes were then probed using the H-22 rabbit polyclonal CDK4 antibody (Santa Cruz), the DCS-22 monoclonal cyclin D3 antibody (Neomarkers) to control the actual expression of the cyclin D3/CDK4 fusion proteins. Anti-mouse immunoglobulin antibody (GE Healthcare) coupled to horseradish peroxidase, was used for detection by enhanced chemiluminescence (Western Lightning, Perkin-Elmer, Boston, Mass.). Various exposures of the bands corresponding to these proteins were collected on Hyperfilms (GE Healthcare).

The pRb kinase assay was performed on the cellular extract bound to the streptavidin-coated sepharose beads (GE Healthcare) as described previously (Coulonval et al., 2003, Exp. Cell Res., 291, 135-149). After 3 hours of incubation at 4° C. with streptavidin-coated sepharose beads, complexes were washed three times with 0.5% NP-40 lysis buffer supplemented with 1 mM DTT and three times with kinase reaction buffer (50 mM Hepes, pH 7.5, 10 mM KCl, 10 mM MgCl2, 2.5 mM EGTA, 1 mM DTT). Washed complexes were resuspended in 40 μl of the kinase reaction buffer containing 2 mM ATP, 0.3 μg of a 48-kDa fragment (amino acid 773-928) of pRb (Sigma), 10 mM β-glycerophosphate, 0.1 mM orthovanadate, 1 mM NaF, 60 μg/ml pefabloc, and 1 μg/ml leupeptine and incubated for 30 min at 30° C. with gentle agitation. Reactions were stopped by adding 60 μl of twice-concentrated Laemmli buffer (glycerol 10%, 60 mM Tris-HCl (pH 6.8), 2% SDS, 100 mM DTT, 50 mM NaF, 100 μM vanadate, and protease inhibitors) and boiling for 5 min. Proteins were resolved by SDS-PAGE and transferred on low fluorescence PVDF membranes (Millipore) before the phosphorylation on T826 of the pRb fragment was detected using a phospho-specific rabbit anti-pRb (T826) antibody (Epitomics). Total GST-Rb load of the membranes was quantitated using the mouse anti-GST antibody (Pierce). Anti-rabbit and anti-mouse immunoglobulin antibodies (Perbio) coupled to Dylight800 and Dylight680 were used for detection on the Odyssey system (Western Lightning; Perkin-Elmer, Boston, Mass.).

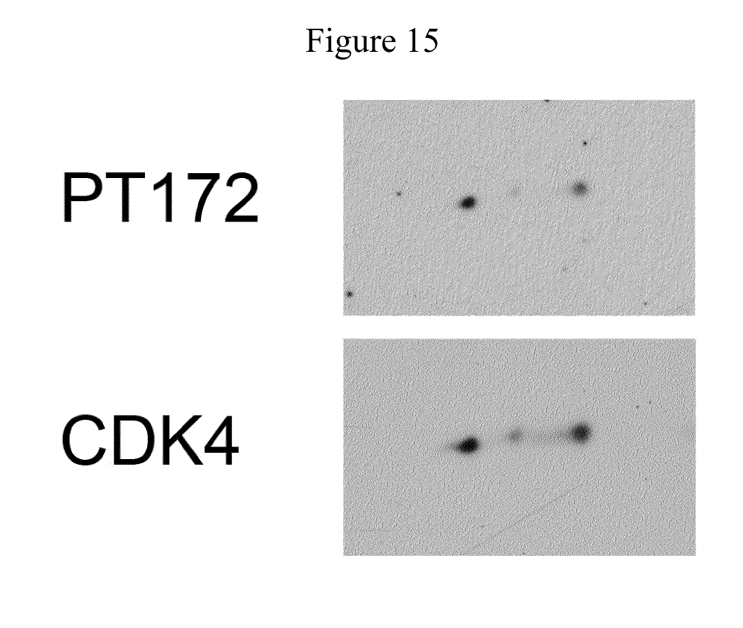

As shown in FIG. 14, the cyclin D3/CDK4 fusion protein can be detected in all the extracts from HEK293T cells transfected with the indicated constructs except those from cells transfected with the constructs driving the luciferase expression used as negative controls. GST-Rb fragment phosphorylation was detected both with the anti-pRb826 antibody or by the shift in apparent molecular weight observed when the western blots are probed with the anti-GST antibody only when it is incubated with extracts from HEK293T cells transfected with the constructs allowing the expression of the wild-type cyclin D3/CDK4 fusion protein.

Next to the wild-type fusion protein, also a mutant cyclin D3/CDK4 fusion protein, with a T172A mutation, was prepared. Entry vector pENTRtopo_D3L3K4T172A (cf. SEQ ID No. 75) was used as a basis to create the final construct for HEK293 cell transfection, by recombination with the destination vector pWFAviIBC, as explained in example 8. The mutant cyclin D3/CDK4T172A fusion protein was amplified using primers defined by SEQ ID No. 76 and 77.

The T172A mutant cyclin D3/CDK4 fusion protein expressed in the HEK293T cells transfected with the pWFAviIBC_D3L3K4T172A construct does not display Rb-kinase activity. Since this result suggests that the contribution to the Rb-kinase activity of the endogenous CDK4 eventually bound to the cyclin D3 part of the Cyclin D3-CDK4 fusion is negligible, the T172A mutant cyclin D3/CDK4 fusion protein can be used as negative control. No difference in Rb-kinase activity is observed between cyclin D3/CDK4 fusion proteins where the cyclin D3 moiety is linked to the CDK4 moiety through a precision non-cleavable (pWFAviIBC_D3L2K4) or cleavable linker (pWFAviIBC_D3L3K4). Alteration in the linker sequence has no impact on the cyclin D3/CDK4 fusion Rb-kinase activity.

### Example 19

### The CDK4 Moiety of a Biotinylatable CCND3/CDK4 Reporter Expressed from the Constructed Lentiviral Vectors Upon Transient Transfection in HEK293T Cells Immobilized on Streptavidin-Coated Beads and Released by Cleavage with Prescission and TEV Proteases is Phosphorylated on T172

In order to verify if the CDK4 fragment of the biotinylatable cyclin D3/CDK4 fusion protein with a linker cleavable by the Prescission protease driven by the lentiviral constructions described in Example 8 is phosphorylated on T172, HEK293T cells (ATCC CRL 11268) were transiently transfected with the corresponding DNA vector produced with the Qiagen midiprep kit following the instructions of the manufacturer. HEK293T cells were cultivated in DMEM medium (InVitrogen) supplemented with pyruvate (1 mM), penicillin (50 U/ml), Streptomycin (50 μg/ml) and 10% foetal calf serum. 6 10⁵ cells were seeded in 6-well plates the day before the transfection by the calcium phosphate method.

On the day of transfection, plasmids (1 μg each) were diluted in 200 μl Tris buffer (Tris 50 mM; EDTA 1 mM) supplemented with 50 μl of 2.5M CaCl₂. This dilution was added dropwise on 250 μl HBS buffer (HEPES 50 mM (pH 7.05); KCl 10 mM; dextrose 12 mM; NaCl 280 mM; Na₂HPO₄ 1.5 mM). After 10 min incubation at 37° C., 250 μl of the mixture was added on the cells. DNA was left on the cells for 6 hours. Thereafter, medium was refreshed and cells were incubated for 48 h in HEK293T cell culture medium supplemented with 0.1 mM biotin. Next, cells were washed with PBS, lysed on ice in 1 ml lysis buffer (100 mM NaCl, 50 mM Tris-HCl pH 7.5, 0.1% SDS, 1% Na deoxycholate, EDTA 1 mM, EGTA 1 mM, 50 mM NaF, 1 mM Orthovanadate, 1 mM β-glycerophosphate, 1 μg/ml leupeptin, 60 μg/ml Pefablock and 10% glycerol). The cellular lysate was incubated at 4° C. for 3 h on streptavidin-Sepharose beads (GE Healthcare) pre-washed with lysis buffer. Beads were washed 3 times with NP-40 buffer supplemented with 1 mM DTT, one time with the Prescission buffer (150 mM NaCl, 50 mM Tris-HCl pH 7.5, EDTA 1 mM, EGTA 1 mM) and digested by Prescission protease (GE Healthcare) overnight at 4° C. under constant mild agitation in 30 μL Prescission digestion buffer (Prescission buffer supplemented with DTT 1 mM and 2 U Precission protease). Beads were then washed once with TEV protease digestion buffer (50 mM Tris-HCl pH 8, EDTA 0.5 mM, DTT 1 mM) and incubated at 37° C. for 30 minutes with 30 μL TEV protease digestion buffer supplemented with DTT 1 mM and 4 Units of TEV protease (InVitrogen). The reaction was stopped by adding an equal volume of a twice concentrated denaturation buffer. Digested proteins were diluted with 300 μl of denaturation buffer (7 M urea, 2 M thiourea, 4% CHAPS, 1% DTE, 1% Pharmalytes 3-10 (GE Healthcare) and 1 mg/ml pefablock) before being resolved by 2D gel electrophoresis separations. Proteins were separated by isoelectric focusing using the Protean IEF cell apparatus from Bio-Rad after active in-gel rehydration on immobilized linear pH gradient (pH 3 to 10) strips (GE Healthcare). After loading onto SDS-polyacrylamide slab gels (12.5%) for separation according to molecular mass, proteins were transferred to PVDF membranes (Perkin Elmer). CDK4 was immunodetected first using an anti-phospho-T172-CDK4 polyclonal rabbit antibody preparation from Cell Signaling Technology (antibody no more commercially available) then using the H-22 polyclonal antibody (Santa Cruz). An anti-rabbit immunoglobulin antibody (GE Healthcare) coupled to horseradish peroxidase, was used for detection by enhanced chemiluminescence (Western Lightning, Perkin-Elmer, Boston, Mass.).

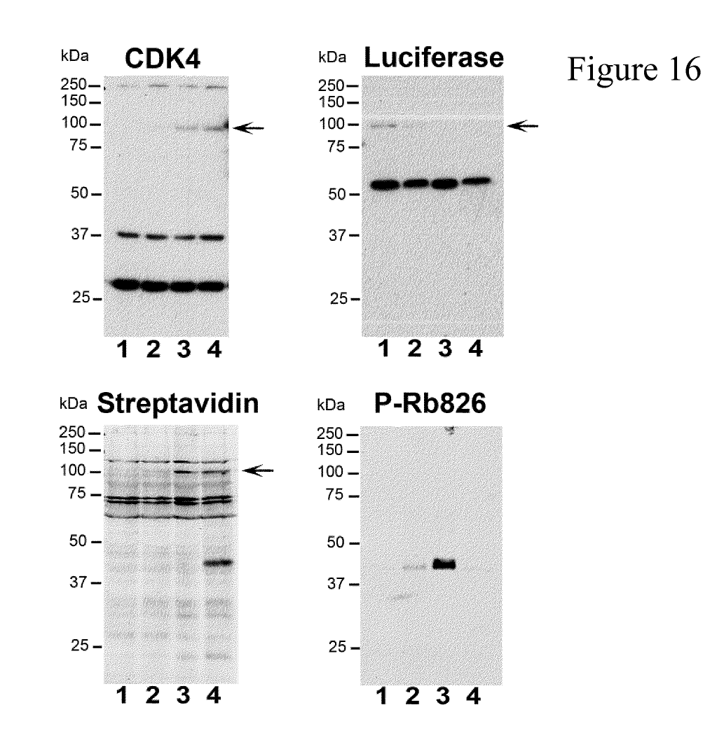

As shown in FIG. 15, the CDK4 fragment of the cyclin D3/CDK4 fusion protein released by Prescission and TEV digestion of streptavidin-sepharose immobilized extracts of HEK293T cells transfected with a vector driving the expression of the BirA biotin ligase and the pWFAviIBC-D3L3K4 vector driving the expression of C-terminal Avi-tagged cyclinD3/CDK4/EGFP fusion protein migrates as discrete spots. Two spots are detected by an antibody recognizing specifically the T172-phosphorylated form. These two spots together with a third one are recognized by an anti-CDK4 antibody. Since our construct includes two TEV cleavage sites between the CDK4 and EGFP parts of the fusion protein, the spot detected only by the CDK4 antibody corresponds to a CDK4 fragment with only one TEV site. The non-phosphorylated CDK4 fragment bearing two TEV sites can not be detected in this assay since it probably migrates outside the range of Pharmalytes strips used (the theoretical isoelectric point is 9.1). The two other spots correspond to acid shifted phosphorylated CDK4 fragments with one or two TEV sites, respectively. A more complex profile is observed when the intact cyclin D3/CDK4 fusion reporter protein is analyzed by 2D gel electrophoresis because of the complex phosphorylation of the cyclin D3 part of the fusion. Nevertheless, significant differences are noticed between the profiles of cyclin D3/CDK4 fusion reporter protein extracted from quiescent cells compared to profiles of fusion extracted from proliferating cells (not shown). Taken together, example 19 suggests that the cyclin D3/CDK4 fusion reporter protein is also phosphorylated on T172 as the endogeneous CDK4. Hence, Prescission digested cyclin D3/CDK4 fusion reporter protein extracted from proliferating cells can be used as immunogen to generate an anti-phospho-CDK4 antibody. Furthermore, cyclin D3/CDK4 fusion reporter protein extracted from quiescent or proliferating cells can be used to identify antibodies recognizing the T172-phosphorylated form of CDK4 and characterize them.

### Example 20

### Only the Wild-Type Prescission-Cleavable Biotinylatable CCND3/CDK4 Reporter Constitutively Expressed in MCF7 Cells Upon Lentiviral Infection Displayed a Rb-Kinase Activity

In order to verify if the Prescission-cleavable biotinylatable cyclin D3/CDK4 fusion protein faithfully reports the activation status of the endogeneous CDK4, lentiviruses were first produced by co-transfecting HEK293T cells with 3 μg of the lentiviral vectors constructed as described in Example 8 and 1.5 and 3 μg of the packaging vectors pMD2.G and pCMVΔR8.2, respectively. pMD2.G (Addgene plasmid #12259) and pCMVΔR8.2 (Addgene plasmid #12263) plasmids were described previously by Pr. Trono's team (University of Lausanne, Switzerland) (Wiznerovicz et al., supra). To this end, these plasmids were first precipitated together with sodium acetate and resuspended in 10 μl of ultrapure DNAse-free water. The DNA mixture was added to 190 μl of Tris buffer (Tris 50 mM; EDTA 1 mM) and 50 μl of 2.5M CaCl₂. This solution was added dropwise on 250μl HBS buffer (HEPES 50 mM (pH 7.05); KCl 10 mM; dextrose 12 mM; NaCl 280 mM; Na₂HPO₄ 1.5 mM). After incubation for 10 min at 37° C., 250 μl of the mixture was added on 6.10⁵ HEK293T cells seeded in two wells of a 6-well plate per construct, the day before transfection (2.5 ml of medium/well). Chloroquine (25 μM) was added in each well before incubation at 36° C. for 6 hours. Thereafter, the medium was refreshed and cells were incubated at 36° C. in 2.5 ml HEK293T cell culture medium per well. After 48 hours, the virus-containing medium was harvested and filtered through a 0.45-μm low protein-binding filter (Millipore, Billerica, Mass., USA). Aliquots were stored at −70° C. Transduction of MCF7 cells done in triplicate, was performed by mixing 10⁴ cells with 200 μl viral supernatant in a 96-well plate. After 72 h, the cells were trypsinized and replicates were pooled in a 24-well plate with fresh MCF7 culture medium. Cell populations were amplified before characterization.

In order to verify if the Prescission-cleavable biotinylatable cyclin D3/CDK4 fusion proteins expressed in MCF7 cells after transduction with the vectors described above could be immobilized on streptavidin-coated sepharose bead and were enzymatically active, the MCF7 cells expressing the luciferase or the cyclin D3/CDK4 fusion protein described above were cultivated in 6 cm Petri dishes with 5 ml DMEM medium supplemented with biotin (0.1 mM), 5% serum, insulin 6 ng/ml, NEAA (GIBCO) 0.1 mM, Sodium pyruvate (1 mM), Penicillin (50 U/ml) and Streptomycin (50 μg/ml). At the end of the culture, cells were washed with PBS and lysed on ice in 400 μL lysis buffer (100 mM NaCl, 50 mM Tris-HCl pH 7.5, 0.1% SDS, 1% Na deoxycholate, EDTA 1 mM, EGTA 1 mM, 50 mM NaF, 1 mM Orthovanadate, 1 mM β-glycerophosphate, 1 μg/ml leupeptin, 60 μg/ml Pefabloc and 10% glycerol) as described in Example 10 and stored at −70° C. Equal amounts of protein from the defrozen lysate were then incubated for 3 h at 4° C. under slight shaking with 5 μl of streptavidin-coated sepharose beads (GE Healthcare) which had been prewashed three times with NP-40 lysis buffer without inhibitor. The pRb kinase assay was performed on the cellular extract bound to the streptavidin-coated sepharose beads (GE Healthcare) as described previously (Coulonval et al., supra). After 3 hours of incubation at 4° C. with streptavidin-coated sepharose beads, complexes were washed three times with 0.5% NP-40 lysis buffer supplemented with 1 mM DTT and three times with the kinase reaction buffer (50 mM Hepes, pH 7.5, 10 mM KCl, 10 mM MgCl2, 2.5 mM EGTA, 1 mM DTT). Washed complexes were resuspended in 40 μl of the kinase reaction buffer containing 2 mM ATP, 0.3 μg of a 48-kDa fragment (aa 773-928) of pRb (Sigma), 10 mM β-glycerophosphate, 0.1 mM orthovanadate, 1 mM NaF, 60 μg/ml pefabloc, and 1 μg/ml leupeptine and incubated for 30 min at 30° C. with continuous gentle agitation. Reactions were stopped by adding 60 μl of twice-concentrated Laemmli buffer (glycerol 10%, 60 mM Tris-HCl (pH 6.8), 2% SDS, 100 mM DTT, 50 mM NaF, 100 μM orthovanadate, and protease inhibitors) and boiling for 5 min. Proteins were resolved by SDS-PAGE and transferred on PVDF membranes (Perkin Elmer). The phosphorylation on T826 of the pRb fragment was detected using a phospho-specific anti-pRb (T826) antibody (Epitomics). An anti-rabbit immunoglobulin antibody (GE Healthcare) coupled to horseradish peroxidase, was used for detection by enhanced chemiluminescence (Western Lightning; Perkin-Elmer, Boston, Mass.).

Transgene expression was assessed by western blotting as described in Example 12. Membranes were probed using the H-22 rabbit polyclonal CDK4 antibody (Santa Cruz), the anti-BirA chicken antibody (Sigma) and the mouse anti-luciferase antibody (Santa Cruz) or the DCS-22 monoclonal cyclin D3 antibody (Neomarkers). Anti-rabbit immunoglobulin antibody (GE Healthcare), anti-chicken antibody (Santa Cruz) or anti-mouse immunoglobulin antibody (GE Healthcare), all coupled to horseradish peroxidase were used for detection by enhanced chemiluminescence (Western Lightning, Perkin-Elmer, Boston, Mass.).

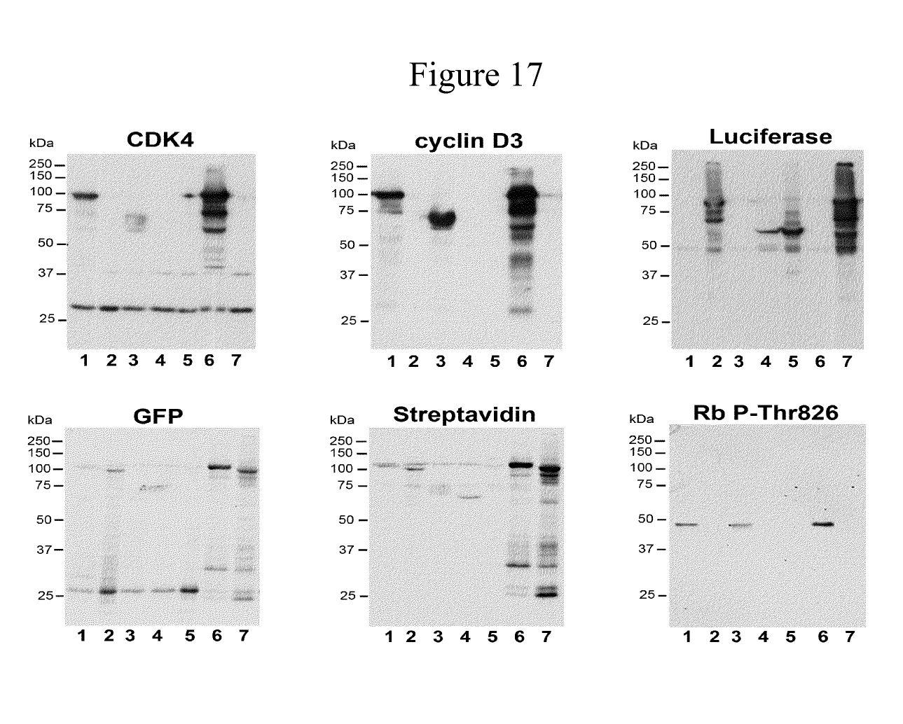

As shown in FIG. 16, stable MCF7 cell populations created by infection with lentiviruses produced with the vectors described in Example 8 expressed in a constitutive way both the wild-type or the T172A mutated Cyclin D3/CDK4 fusion proteins. This expression could be detected both with the anti-CDK4, antiCCND3 and anti-GFP antibodies. Both cell lines also express detectable BirA/mCherry fusion protein. Accordingly, both fusion proteins are biotinylated as detected by AlexaFluor680-coupled streptavidin. Hence, a significant Rb-kinase activity can be immobilized on streptavidin-sepharose beads as judged by the phosphorylation of a GST-Rb substrate. Furthermore, this Rb kinase activity was only detected in extracts of MCF7 cells expressing the wild-type Cyclin D3/CDK4 fusion protein. Extracts of cells expressing the T172A-mutated CDK4 or the luciferase reporters were lacking Rb kinase activity.

Example 20 thus shows that Prescission-cleavable biotinylatable cyclin D3/CDK4 fusion protein expressed in MCF7 after transduction with the appropriate vectors described above could be purified with streptavidin-coated sepharose beads and remained enzymatically active. Furthermore, only beads incubated with extracts from MCF7 cells expressing a wild type biotinylatable Cyclin D3/CDK4 fusion possessed detectable Rb kinase activity. Biotinylated Avi-tagged Cyclin D3/CDK4 fusion protein expressed in a stable cell population of MCF7 cells after transduction with the vectors described in Example 8 is thus a perfect reporter molecule of the activation status of endogenous CDK4 described in Example 11.

The MCF7 cell-lines stably transfected with the Prescission-cleavable biotinylated wild type D3L3K4 fusion and BirA/Cherry (called MCF7-AZ-V955) was deposited on Sep. 4, 2013 at the Belgian Coordinated Collection of Microorganisms (BCCM), Ghent Belgium, under the provisional number LMBP 10330CB.

### Example 21

### CCND3/CDK4 Reporters with an Internal Avi-Tag Sequence are Expressed from the Constructed Lentiviral Vectors Upon Transient Transfection in HEK293T Cells, Immobilizable on Streptavidin-Coated Beads and Enzymatically Active

In order to verify if the Avi-tag of cyclin D3/CDK4 fusion reporter proteins is also functional when inserted within the reporter sequence instead of at its C-terminal end, HEK293T cells (ATCC CRL 11268) were transiently transfected with the indicated DNA vectors produced with the Qiagen midiprep kit following the instructions of the manufacturer. HEK293T cells were cultivated in DMEM medium (Invitrogen) supplemented with pyruvate (1 mM), penicillin (50 U/ml), Streptomycin (50 μg/ml) and 10% foetal calf serum. 6 10⁵ cells were seeded in 6-well plates the day before the transfection by the calcium phosphate method. Cells were transfected with vectors encoding the different cyclin D3/CDK4 fusion reporters or reporters in which the cyclin D3/CDK4 fusion was replaced by a luciferase reporter together with a vector driving the expression of the BirA biotin ligase.

On the day of transfection, plasmids (1 μg each) were diluted in 200 μl Tris buffer (Tris 50 mM; EDTA 1 mM) supplemented with 50 μl of 2.5M CaCl₂. This dilution was added dropwise on 250 μl HBS buffer (HEPES 50 mM (pH 7.05); KCl 10 mM; dextrose 12 mM; NaCl 280 mM; Na₂HPO₄ 1.5 mM). After 10 min incubation at 37° C., 250 μl of the mixture was added on the cells. DNA was left on the cells for 6 hours. Thereafter, medium was refreshed and cells were incubated for 48 h in HEK293T cell culture medium supplemented with 0.1 mM biotin. Lysates were diluted with twice-concentrated Laemmli buffer (glycerol 10%, 60 mM Tris-HCl (pH 6.8), 2% SDS, 100 mM DTT, 50 mM NaF, 100 μM orthovanadate, and protease inhibitors) and proteins were resolved by SDS-PAGE and transferred onto PVDF membranes (Perkin Elmer) or low fluorescence PVDF membranes (Millipore) for chemiluminescence or Odyssey detection, respectively. Membranes were then probed using the anti-BirA chicken antibody (Sigma) and the mouse anti-luciferase antibody (Santa Cruz). Anti-chicken immunoglobulin (Santa Cruz) or anti-rabbit immunoglobulin antibody (GE Healthcare), both coupled to horseradish peroxidase, were used for detection by enhanced chemiluminescence (Western Lightning, Perkin-Elmer, Boston, Mass.). Various exposures of the bands corresponding to these proteins were collected on Hyperfilms (GE Healthcare). Mouse anti-GFP monoclonal antibody (Santa Cruz) was detected with anti-mouse immunoglobulin antibody (Perbio) coupled to Dylight800 with the Odyssey system (Western Lightning; Perkin-Elmer, Boston, Mass.). Biotinylated proteins were detected in parallel with AlexaFluor 680-labelled streptavidin (InVitrogen) using the Odyssey system.

The pRb kinase assay was performed on the cellular extract bound to the streptavidin-coated sepharose beads (GE Healthcare) as described previously (Coulonval et al., supra). After 3 hours of incubation at 4° C. with streptavidin-coated sepharose beads, complexes were washed three times with 0.5% NP-40 lysis buffer supplemented with 1 mM DTT and three times with the kinase reaction buffer (50 mM Hepes, pH 7.5, 10 mM KCl, 10 mM MgCl2, 2.5 mM EGTA, 1 mM DTT). Washed complexes were resuspended in 40 μl of the kinase reaction buffer containing 2 mM ATP, 0.3 μg of a 48-kDa fragment (aa 773-928) of pRb (Sigma), 10 mM β-glycerophosphate, 0.1 mM orthovanadate, 1 mM NaF, 60 μg/ml pefabloc, and 1 μg/ml leupeptine and incubated for 30 min at 30° C. with continuous gentle agitation. Reactions were stopped by adding 60 μl of twice-concentrated Laemmli buffer (glycerol 10%, 60 mM Tris-HCl (pH 6.8), 2% SDS, 100 mM DTT, 50 mM NaF, 100 μM orthovanadate, and protease inhibitors) and boiling for 5 min. Proteins were resolved by SDS-PAGE and transferred on PVDF membranes (Perkin elmer). The phosphorylation on T826 of the pRb fragment was detected using a phospho-specific anti-pRb (T826) antibody (Epitomics). An anti-rabbit immunoglobulin antibody (GE Healthcare) coupled to horseradish peroxidase, was used for detection by enhanced chemiluminescence (Western Lightning; Perkin-Elmer, Boston, Mass.).

As shown in FIG. 17, all transgenes were expressed in HEK293T cells. As all tested constructs contained a BirA/mCherry fusion following via an IRES sequence the expression module of the Avi-tagged transgene, biotinylation of the trangenes was expected. The panel noted BirA confirms the expression of the BirA/mCherry fusion protein. Expression of luciferase was observed only in HEK293T cells transfected with the vectors driving the expression of the luciferase or the Avi-tagged luciferase/EGFP fusion. EGFP-Fusion proteins were detected in all cell extracts of HEK293T except the extracts from control cells, and from cells transfected with the vector driving the expression of the luciferase reporter alone or of the Avi-tagged reporters lacking the GFP moiety. All the Avi-tagged fusion proteins are biotinylated as revealed by incubation with streptavidin coupled to dye-light800. Rb-kinase activity was only detected in extracts of HEK293T cells transfected with the plasmids driving the expression of the CyclinD3/CDK4 fusion reporter proteins irrespective of the presence of the GFP fragment in the fusion. Example 21 thus indicates that the internal localization of the Avi-tag does not preclude the biotinylation and immobilization on streptavidin-coated matrices of the Avi-tagged reporters. Furthermore, the presence of a GFP marker in the reporter downstream of the Avi-tagged cyclinD3/CDK4 fusion does not impede its enzymatic activity.

### Example 22

### The Composition of Lysis Buffers Influences the Recovery of Rb-Kinase Activity of Biotinylated Avi-Tagged Cyclin D3/CDK4 Fusion Expressed in Stable Cell Populations HCT116K7AS Cell Extracts Immobilized on Streptavidin Sepharose

In order to identify a lysis buffer able to recover most of the Rb-kinase activity of biotinylatable Cyclin D3/CDK4 fusion expressed in quiescent or serum-stimulated HCT116K7AS cells transduced with the vectors described in Example 8, the HCT116K7AS cells expressing the cyclin D3/CDK4 fusion protein described in the Example 14 were cultivated in 10 cm Petri dishes with 10 ml DMEM medium supplemented with 10% serum, Sodium pyruvate (1 mM), Penicillin (50 U/ml) and Streptomycin (50 μg/ml). These cells express only a mutated version of CDK7 inhibitable by bulky ATP analogs such as 1-NMPP1 (CDK7AS). The cells were rendered quiescent by incubating them for three days in serum-free DMEM supplemented with biotin (0.1 mM), Sodium pyruvate (1 mM), Penicillin (50 U/ml) and Streptomycin (50 μg/ml). Cells were stimulated to proliferate by adding 10% serum for 16 hours in the continuous presence of biotin (0.1 mM). At the end of the culture, cells were washed with PBS and lysed on ice in 1 ml of the lysis buffers described in Table 6. The cellular lysate were further or not homogenized (glass/glass) and/or sonicated 3 times before being stored at −70° C. Equal amounts of protein (corresponding to one third of the total lysate volume) from the defrozen lysate were then incubated for 3 h at 4° C. under slight shaking with 20 μl of streptavidin-coated sepharose beads (GE Healthcare) which had been prewashed three times with NP-40 lysis buffer without inhibitors. The pRb kinase activity of the lysates was assayed on the cellular extract bound to the streptavidin-coated sepharose beads as described previously (Coulonval et al., supra). After 3 hours of incubation at 4° C. with streptavidin-coated sepharose beads, complexes were washed three times with 0.5% NP-40 lysis buffer supplemented with 1 mM DTT and three times with the kinase reaction buffer (50 mM Hepes, pH 7.5, 10 mM KCl, 10 mM MgCl2, 2.5 mM EGTA, 1 mM DTT). Washed complexes were resuspended in 40 μl of the kinase reaction buffer containing 2 mM ATP, 0.3 μg of a 48-kDa fragment (aa 773-928) of pRb (Sigma), 10 mM β-glycerophosphate, 0.1 mM orthovanadate, 1 mM NaF, 60 μg/ml pefabloc, and 1 μg/ml leupeptine and incubated for 30 min at 30° C. with continuous gentle agitation. Reactions were stopped by adding 60 μl of twice-concentrated Laemmli buffer (glycerol 10%, 60 mM Tris-HCl (pH 6.8), 2% SDS, 100 mM DTT, 50 mM NaF, 100 μM orthovanadate, and protease inhibitors) and boiling for 5 min. Proteins were resolved by SDS-PAGE and transferred on PVDF membranes (Perkin Elmer). The phosphorylation on T826 of the pRb fragment was detected using a phospho-specific anti-pRb (T826) antibody (Epitomics). An anti-rabbit immunoglobulin antibody (GE Healthcare) coupled to horseradish peroxidase, was used for detection by enhanced chemiluminescence (Western Lightning; Perkin-Elmer, Boston, Mass.).

TABLE 6
Buffer compositions used:

| Buffer 1 | Buffer 2 | Buffer 3 | Buffer 4 | Buffer 5 | Buffer 6 |
|---|---|---|---|---|---|
| Tris pH 7.5 | ″ | ″ | ″ | ″ | ″ |
| 50 mM | | | | | |
| NaCl | NaCl | NaCl | NaCl | NaCl | NaCl |
| 150 mM | 100 mM | 100 mM | 100 mM | 100 mM | 100 mM |
| NP40 0.5% | NP40 1% | NP40 1% | NP40 1% | NP40 1% | NP40 1% |
| | | SDS 0.1% | SDS 0.1% | SDS 0.6% | SDS 0.6% |
| | | | Na | | Na |
| | | | Deoxy- | | Deoxy- |
| | | | cholate | | cholate |
| | | | 1% | | 1% |
| | EDTA | EDTA | EDTA | EDTA | EDTA |
| | 1 mM | 1 mM | 1 mM | 1 mM | 1 mM |
| | EGTA | EGTA | EGTA | EGTA | EGTA |
| | 1 mM | 1 mM | 1 mM | 1 mM | 1 mM |
| NaF 50 mM | EGTA | EGTA | EGTA | EGTA | EGTA |
| | 1 mM | 1 mM | 1 mM | 1 mM | 1 mM |
| DTT 1 mM | EGTA | EGTA | EGTA | EGTA | EGTA |
| | 1 mM | 1 mM | 1 mM | 1 mM | 1 mM |
| 10% glycerol | EGTA | EGTA | EGTA | EGTA | EGTA |
| | 1 mM | 1 mM | 1 mM | 1 mM | 1 mM |
| Na ortho- | EGTA | EGTA | EGTA | EGTA | EGTA |
| vanadate | 1 mM | 1 mM | 1 mM | 1 mM | 1 mM |
| 1 mM | | | | | |
| B glycérol- | EGTA | EGTA | EGTA | EGTA | EGTA |
| phosphate | 1 mM | 1 mM | 1 mM | 1 mM | 1 mM |
| 1 mM | | | | | |
| Leupeptin | EGTA | EGTA | EGTA | EGTA | EGTA |
| 1 ug/ml | 1 mM | 1 mM | 1 mM | 1 mM | 1 mM |
| Pefabloc | EGTA | EGTA | EGTA | EGTA | EGTA |
| 60 ug/ml | 1 mM | 1 mM | 1 mM | 1 mM | 1 mM |

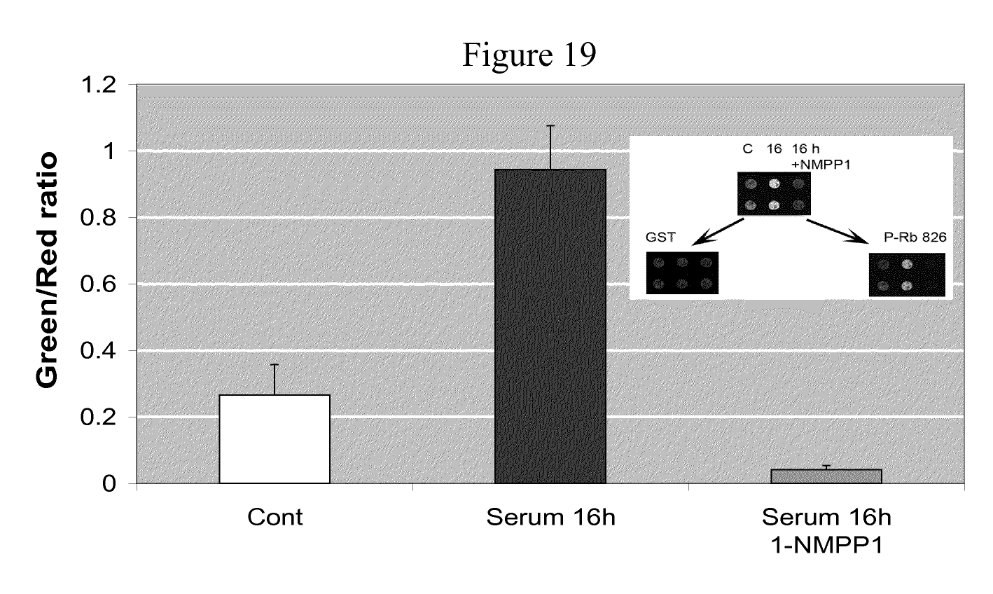

As shown in FIG. 18, Buffer 1 to 4 allow to recover the Rb kinase activity of the exogeneous biotinylatable Cyclin D3/CDK4 fusion expressed in HCT116K7AS cells induced by serum even without glass/glass homogenization and/or sonication. Buffer 5 to 6 do not allow to recover any Rb kinase activity. The highest recovery of Rb kinase activity even without glass/glass homogenization and/or sonication is achieved in buffer 4 although the best discrimination between control and serum stimulated reporter Rb-kinase activity is seen with buffer 1.

Example 22 shows that appropriate buffers are available to extract the biotinylated Avi-tagged Cyclin D3/CDK4 fusion protein Rb-kinase of HCT116K7AS transduced with the vectors described in Example 8 without the need to homogenize or sonicate the samples.

Example 22 thus brings the proof of concept that the enzymatic Rb kinase activity of a reporter molecule comprising a cyclin D3/CDK4 fusion protein coupled to an Avi-tag and expressed together with BirA/mCherry fusion can be extracted without mechanical handling and immobilized on streptavidin-coated supports. The extraction conditions of the enzymatic Rb kinase activity of this reporter molecule are thus compatible with the use of the reporter in miniaturized high/medium throughput screening.

### Example 23

### Alterations Upon Serum Challenge of the Cell Extract Rb-Kinase Activity Immobilized on Streptavidin Sepharose of Biotinylated Avi-Tagged Cyclin D3/CDK4 Fusion Expressed in Stable Cell Populations HCT116K7AS can be Detected by a Dot-Blot Technology

In order to identify a detection method amenable to high throughput miniaturization to determine the Rb-kinase activity of an exogeneous biotinylatable Cyclin D3/CDK4 reporter expressed in quiescent or serum-stimulated HCT116K7AS cells transduced with the vectors described in Example 8, the HCT116K7AS cells expressing the cyclin D3/CDK4 fusion protein described in the Example 14 were cultivated in 10 cm Petri dishes with 10 ml DMEM medium supplemented with 10% serum, Sodium pyruvate (1 mM), Penicillin (50 U/ml) and Streptomycin (50 μg/ml). These cells express only a mutated version of CDK7 inhibitable by bulky ATP analogs such as 1-NMPP1 (CDK7AS). The cells were rendered quiescent by incubating them for three days in DMEM without serum, but supplemented with biotin (0.1 mM), Sodium pyruvate (1 mM), Penicillin (50 U/ml) and Streptomycin (50 μg/ml). Cells were stimulated to proliferate by adding 10% serum for 16 hours in the continuous presence of biotin (0.1 mM). At the end of the culture, cells were washed with PBS and lysed on ice in 100 μL of the NP-40 lysis buffer. 100 μL of the defrozen lysate were then incubated for 3 h at 4° C. under slight shaking with 20 μl of streptavidin-coated sepharose beads (GE Healthcare) which had been prewashed three times with NP-40 lysis buffer without inhibitors. The pRb kinase activity of the lysates assay was assayed on the cellular extract bound to the streptavidin-coated sepharose beads as described previously (Coulonval et al., supra). After 3 hours of incubation at 4° C. with streptavidin-coated sepharose beads, complexes were washed three times with 0.5% NP-40 lysis buffer supplemented with 1 mM DTT and three times with the kinase reaction buffer as described in Example 22. Washed complexes were resuspended in 40 μl of the kinase reaction buffer containing 2 mM ATP, 0.3 μg of a 48-kDa fragment (aa 773-928) of pRb (Sigma), 10 mM β-glycerophosphate, 0.1 mM orthovanadate, 1 mM NaF, 60 μg/ml pefabloc, and 1 μg/ml leupeptine and incubated for 30 min at 30° C. with occasional gentle agitation. Reactions were stopped by adding 400 μl of ice-cold TBS buffer (NaCl 0.5M; Tris 10 mM pH 7.5). Proteins were transferred on PVDP membranes using a Minifold-1 Dot-blot system (Schleicher and Schüell) before the phosphorylation on T826 of the pRb fragment was detected using a phospho-specific rabbit anti-pRb (T826) antibody (Epitomics,). Total GST-Rb load of the membranes was quantitated using the mouse anti-GST antibody (Pierce). An anti-rabbit and anti-mouse immunoglobulin antibody (Perbio) coupled to Dylight800 and Dylight680 were used for detection on the Odyssey system (Western Lightning; Perkin-Elmer, Boston, Mass.).

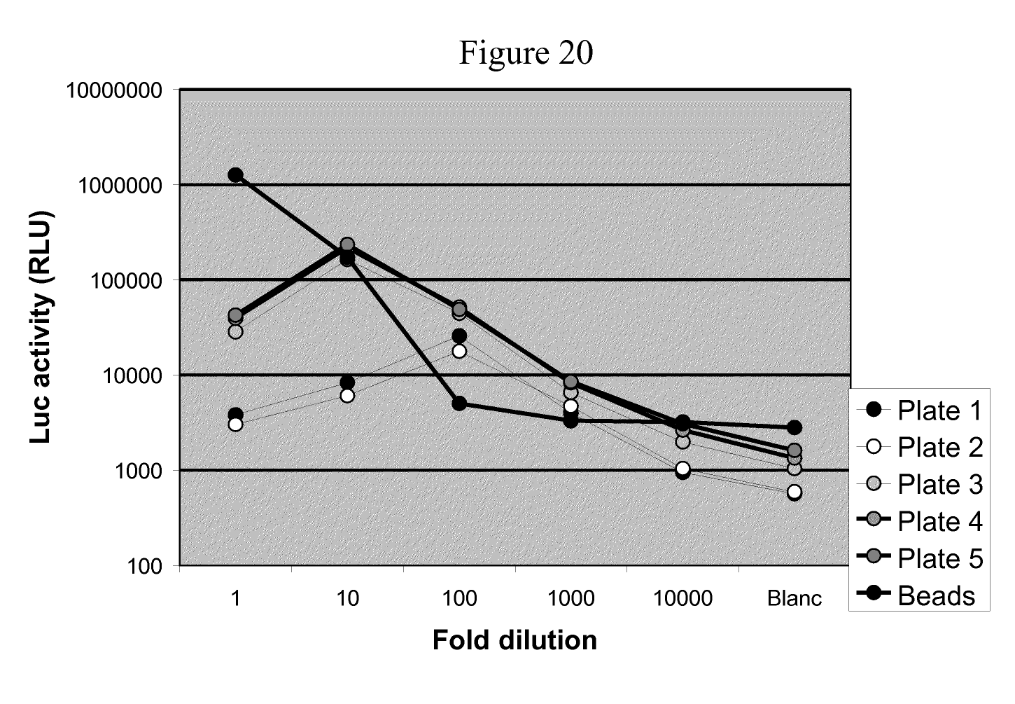

As shown in FIG. 19, the alterations in GST-Rb phosphorylation status can be detected by a dot blot approach when the GST-Rb substrate is incubated with streptavidin-sepharose immobilized cellular extracts of HCT116K7AS cells expressing an exogeneous biotinylatable Cyclin D3/CDK4 reporter stimulated with serum with or without the CDK7 inhibitor 1-NMPP1.

Example 23 shows that a dot-blot technology is usable to detect the Rb-kinase activity of extracts of HCT116K7AS cells expressing a biotinylated Avi-tagged Cyclin D3/CDK4 fusion protein reporter after its immobilization on streptavidin sepharose.

Example 23 thus brings the proof of concept that the enzymatic Rb kinase activity of a reporter molecule comprising a cyclin D3/CDK4 fusion protein coupled to an Avi-tag and expressed together with BirA/mCherry fusion can be extracted without mechanical handling, immobilized on streptavidin-coated supports and detected by a method compatible with the use of the reporter in miniaturized high/medium throughput screening.

### Example 24

### Streptavidin Sepharose Beads have a Better Capacity to Immobilize Biotinylated Avi-Tagged Luciferase/GFP Fusion Expressed in HEK293T Cells

In order to identify which support immobilizes linearly increasing amounts of exogeneous biotinylatable luciferase reporter, HEK293T cells (ATCC CRL 11268) were cultivated and transiently transfected with a plasmid encoding a non-biotinylatable luciferase reporter (pDLVCTEGFPTetOV5His luc) or a biotinylatable luciferase reporter together with a BirA/mCherry fusion protein (pWFAviIBC_Luc) as described in Example 12.

After transfection, cells were washed with PBS and lysed on ice in 1 ml NP-40 lysis buffer as described in Example 10. The homogenized (glass/glass) cellular lysate was sonicated 3 times and stored at −20° C. After normalization of the luciferase activity of the defrozen lysates, lysate with the biotinylatable luciferase reporter were diluted 5 times by tenfold steps in the lysate with the non-biotinylatable luciferase reporter. 50 μl of the mixed lysates were then incubated for 3 h at 4° C. under slight shaking with 5 μl of streptavidin-coated sepharose beads (GE Healthcare) which had been prewashed three times with NP-40 lysis buffer without inhibitors. Alternatively, 50 μl of mixed lysates were added on either Perkin Elmer or Pierce streptavidin-coated plates as well as on Pierce neutravidin-coated plates (. After 3 h, the supernatant was collected, hen beads and plates bound with transgene were washed three times with cold NP-40 lysis buffer before enzymatic activities is measured.

For the luciferase assay, two 20 μl samples of the supernatant and the resuspended washed bead (bound luciferase activity) were spotted in a black 96 well plate for luciferase assay. Luciferase assay was started by adding 20 μl of luciferase reaction buffer (Tricine 40 mM; (MgCO₃)₄Mg(OH)₂.5H₂O 2.14 mM; MgSO₄.7H₂O 5.34 mM; DTT 66.6 mM; EDTA 0.2 mM; coenzyme A (40 mg/50 ml); ATP 0.7 mM; luciferin 940 μM) and recording the emitted light for 1 sec in a Berthold luminometer. Luciferase activity bound was analyzed by adding 20 μl of luciferase reaction buffer (Tricine 40 mM; (MgCO₃)₄Mg(OH)₂.5H₂O 2.14 mM; MgSO₄.7H₂O 5.34 mM; DTT 66.6 mM; EDTA 0.2 mM; coenzyme A (40 mg/50 ml); ATP 0.7 mM; luciferin 940 μM) and recording the emitted light for 1 sec in a Berthold luminometer.

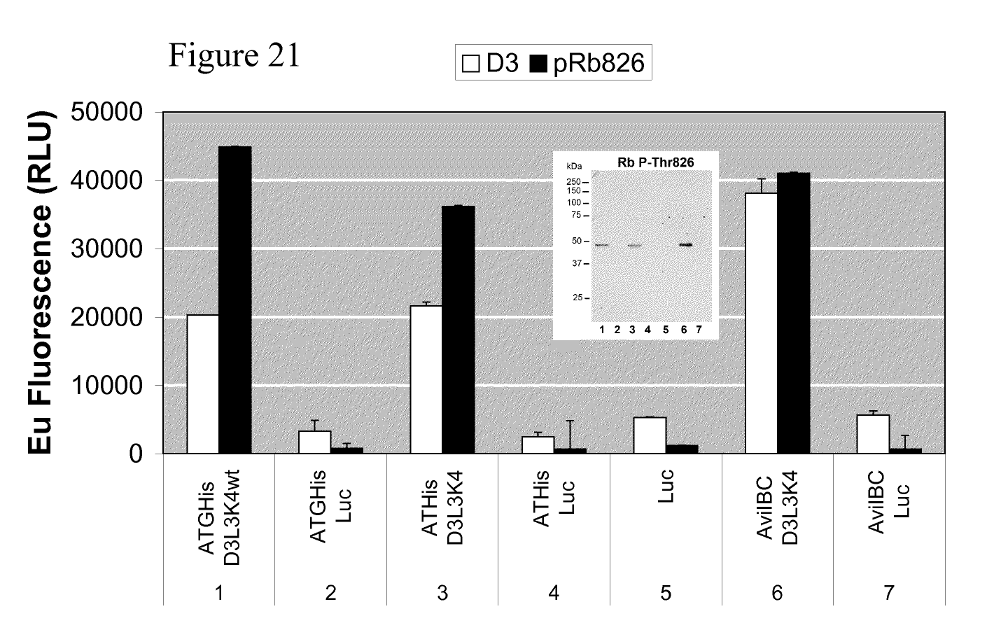

As shown in FIG. 20, saturation of the plates occurs at high concentration of cellular extracts while streptavidin beads capacity to immobilize biotinylated luciferase is higher when protein concentration is higher.

Example 24 shows that streptavidin beads is the support of choice to immobilize biotinylated reporters when their expression is high. With lower expression levels, Neutravidin coated plates are also adequate.

### Example 25

### The Cell Extract Rb-Kinase Activity Immobilized on Streptavidin Sepharose of Biotinylated Avi-Tagged Cyclin D3/CDK4 Fusion Expressed in Transfected HEK293T Cells can be Detected by a DELFIA Assay

In order to identify a more sensitive detection method amenable to high throughput miniaturization to determine the Rb-kinase activity of an exogeneous biotinylatable Cyclin D3/CDK4 reporter expressed in mammalian cells, HEK293T cells (ATCC CRL 11268) were transiently transfected with DNA vectors driving the expression of biotinylatable Cyclin D3/CDK4 or luciferase reporters fused or not to GFP. Transfected DNA was produced with the Qiagen midiprep kit following the instructions of the manufacturer.

HEK293T cells were cultivated in DMEM medium (Invitrogen) supplemented with pyruvate (1 mM), penicillin (50 U/ml), Streptomycin (50 μg/ml) and 10% foetal calf serum. 6 10⁵ cells were seeded in 6-well plates the day before the transfection by the calcium phosphate method. Cells were transfected with vectors encoding the different cyclin D3/CDK4 fusion reporters or reporters in which the cyclin D3/CDK4 fusion was replaced by a luciferase reporter together with a vector driving the expression of the BirA biotine ligase.

On the day of transfection, plasmids (1 μg each) were diluted in 200 μl Tris buffer (Tris 50 mM; EDTA 1 mM) supplemented with 50 μl of 2.5M CaCl₂. This dilution was added dropwise on 250 μl HBS buffer (HEPES 50 mM (pH 7.05); KCl 10 mM; dextrose 12 mM; NaCl 280 mM; Na₂HPO₄ 1.5 mM). After 10 min incubation at 37° C., 250 μl of the mixture was added on the cells. DNA was left on the cells for 6 hours. Thereafter, medium was refreshed and cells were incubated for 48 h in HEK293T cell culture medium supplemented with 0.1 mM biotin. Then, cells were lysed in buffer 4 as described in Example 22.

The DELFIA Rb-kinase assay was started by adding 5 μl streptavidin-coated sepharose beads prewashed twice with NP40 lysis buffer on 100 μl of cellular lysate loaded in PALL Acropreps plates and incubating the plate for 2 hours at 4° C. Exogeneous streptavidin-bound biotinylatable Cyclin D3/CDK4 reporter was next resuspended in 40 μl of a kinase reaction buffer containing 2 mM ATP, 0.3 μg of a 48-kDa fragment (aa 773-928) of pRb (Sigma), 10 mM β-glycerophosphate, 0.1 mM orthovanadate, 1 mM NaF, 60 μg/ml pefabloc, and 1 μg/ml leupeptine and further incubated for 30 minutes at 37° C. under mild shaking. The Rb-kinase assay was stopped after 30 minutes by adding 10 μl of EDTA 120 mM to the reaction mixture. The supernatant of the bead suspension was transferred by centrifugation to Nunc immobilizer Gluthatione F96 clear plates (Nunc) together with 50 μl DELFIA Assay buffer (Perkin Elmer) supplemented with 0.1% BSA to capture the GST-Rb fragment used as substrate in the Rb-kinase assay. Plates were incubated under mild shaking 1 hour at room temperature before being washed twice with DELFIA wash solution (Perkin Elmer). Plates were then incubated overnight at 4° C. under slight agitation (Vibrax shacker) with 50 μl DELFIA Assay buffer (Perkin Elmer) supplemented with 0.1% BSA and 1/1000 anti P-RB826 antibody (Epitomics). Plates were next washed 4 times with DELFIA wash solution (Perkin Elmer) before being incubated 10 minutes at room temperature with 50 μl DELFIA Assay buffer (Perkin Elmer) supplemented with 0.1% BSA and 300 ng/ml Eu-labelled anti rabbit IGg (Perkin Elmer). Plates were next washed 4 times with DELFIA wash solution (Perkin Elmer) before being incubated 5 at room temperature with DELFIA enhancement solution (Perkin Elmer). Fluorescence of release Eu-cryptates was measured after a delay of 100μ seconds for 400μ seconds on a TECAN Infinite Pro 2000 fluorimeter equipped with a 340 nm excitation filter and a 610 nm emission filter.

The remaining streptavidin beads were resuspended in PALL Acropreps plates with 50 μl DELFIA Assay buffer (Perkin Elmer) supplemented with 0.1% BSA. Plates were incubated overnight at 4° C. under slight agitation (Vibrax shacker) with 50 μl DELFIA Assay buffer (Perkin Elmer) supplemented with 0.1% BSA and 1/1000 of the DCS-28 monoclonal cyclin D3 antibody (Santa Cruz). Plates were next washed 4 times with DELFIA wash solution (Perkin Elmer) before being incubated 2 hours at room temperature with 50 μl DELFIA Assay buffer (Perkin Elmer) supplemented with 0.1% BSA and 300 ng/ml Eu-labelled anti mouse IGg (Perkin Elmer). Plates were next washed 4 times with DELFIA wash solution (Perkin Elmer) before being incubated 5 min under mild shaking at 5 at room temperature with DELFIA enhancement solution (Perkin Elmer). The supernatant of the bead suspension was transferred by centrifugation to Perkin Elmer white fluorescence plates. Fluorescence of release Eu-cryptates was measured after a delay of 100μ seconds for 400μ seconds on a TECAN Infinite Pro 2000 fluorimeter equipped with a 340 nm excitation filter and a 610 nm emission filter.

The Rb-kinase assay was performed in parallel in the classic way on the cellular extract bound to the streptavidin-coated sepharose beads (GE Healthcare) as described previously (Coulonval et al., supra) and in the Example 21.

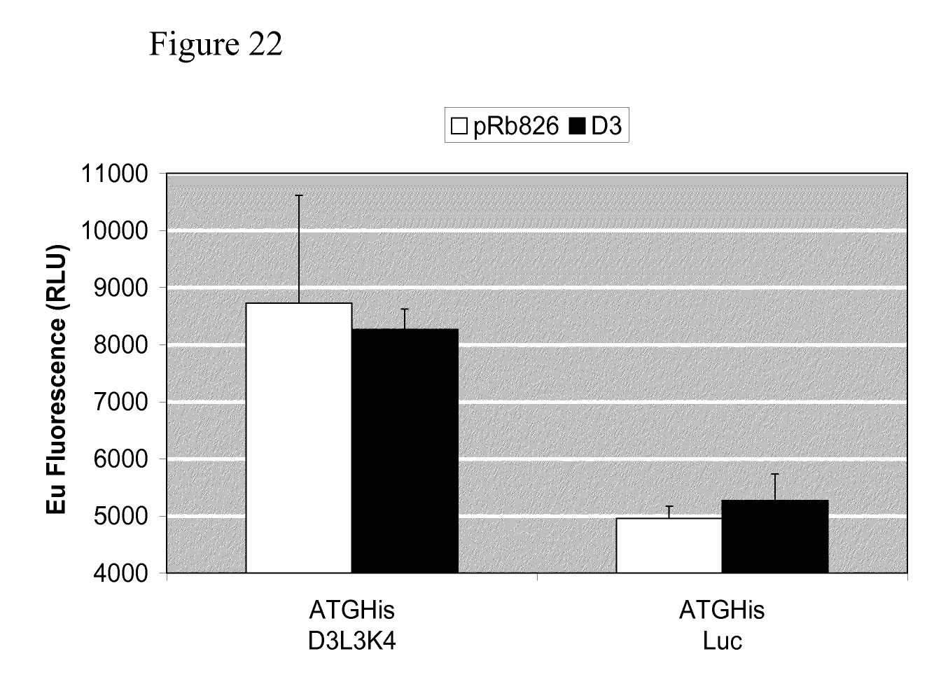

As shown in FIG. 21, Eu cryptate fluorescence is only detected with a Eu-labeled secondary antibody and an anti-P-Rb antibody in the Rb-kinase reaction products immobilized on gluthation-coated plates obtained with extracts of HEK293T cells transfected with vectors allowing the expression of the BirA biotin ligase fused or not to mCherry and of biotinylatable cyclin D3/CDK4 fusion reporters. No phosphorylation of the GST-Rb substrate immobilized on the gluthation plates is detected with extracts of HEK293T cells transfected with vectors allowing the expression of the BirA biotin ligase fused or not to mCherry and of biotinylatable luciferase fusion reporters. Eu cryptate fluorescence is also directly detected with a Eu-labeled secondary antibody and an anti-cyclin D3 antibody on the streptavidin-sepharose bead-bound extracts of HEK293T cells transfected with vectors allowing the expression of the BirA biotin ligase fused or not to mCherry and of biotinylatable cyclin D3/CDK4 fusion reporters.

Example 25 brings thus the proof of concept that the DELFIA technology is thus applicable to detect Rb-kinase activity and cyclin D3/CDK4 fusion presence in extracts immobilized on streptavidin-sepharose of transfected HEK293T cells. The indirect measurement of the activation of the CDK4 part of the cyclin D3/CDK4 fusion by a Rb-kinase assay is possible by the DELFIA technology and amenable to high throughput scale. Once a suitable phospho-T172 specific anti-CDK4 antibody will be available, direct quantification of the activation of the CDK4 part of the cyclin D3/CDK4 fusion will also be possible with the DELFIA method.

### Example 26

### The Cell Extract Rb-Kinase Activity Immobilized on Streptavidin-Coated Plates of Biotinylated Internally Avi-Tagged Cyclin D3/CDK4 Fusion Expressed in MCF7 Cells can be Detected by a DELFIA Assay

In order to verify if the DELFIA technology is sensitive enough to detect Rb-kinase activity and transgene expression of MCF7 cells cultivated in 96-well plates and expressing an inducible biotinylatable cyclin D3/CDK4 fusion reporter, lentiviruses were prepared to infect MCF7 cells. To this end, HEK293T cells were co-transfected with 3 μg of the lentiviral vectors constructed as described in Example 8 and 1.5 and 3 μg of the packaging vectors pMD2.G and pCMVΔR8.2, respectively. pMD2.G (Addgene plasmid #12259) and pCMVΔR8.2 (Addgene plasmid #12263) plasmids were described previously by Pr. Trono's team (University of Lausanne, Switzerland) (Wiznerovicz et al., supra). To this end, all plasmids were first precipitated together with sodium acetate and resuspended in 10 μl of ultrapure DNAse-free water. The DNA mixture was added to 190 μl of Tris buffer (Tris 50 mM; EDTA 1 mM) and 50 μl of 2.5M CaCl₂. This solution was added dropwise on 250 μl HBS buffer (HEPES 50 mM (pH 7.05); KCl 10 mM; dextrose 12 mM; NaCl 280 mM; Na₂HPO₄ 1.5 mM). After incubation for 10 min at 37° C., 250 μl of the mixture was added on 6.10⁵ HEK293T cells seeded in two wells of a 6-well plate per construct, the day before transfection (2.5 ml of medium/well). Chloroquine (25 μM) was added in each well before incubation at 36° C. for 6 hours. Thereafter, the medium was refreshed and cells were incubated at 36° C. in 2.5 ml HEK293T cell culture medium per well. After 48 hours, the virus-containing medium was harvested and filtered through a 0.45-mm low protein-binding filter (Millipore, Billerica, Mass., USA). Aliquots were stored at −70° C. Transduction of MCF7 cells done in triplicate, was performed by mixing 10⁴ cells with 200 μl viral supernatant in a 96-well plate. After 72 h, the cells were trypsinized and replicates were pooled in a 24-well plate with fresh MCF7 culture medium. Populations of MCF7 cells with inducible biotinylatable internally Avi-tagged Prescission-cleavable cyclin D3/CDK4 fusion protein or luciferase reporters fused or not to GFP were generated by successive lentiviral infection. MCF7 cells were first infected with a lentivirus prepared with a vector driving the expression of the rTTA3 regulator together with the DsRed fluorescent marker. The MCF7-rTTA3 cell population was amplified and enriched in red fluorescent cells on an Altra Cell Sorter (Beckton Dickinson). This purified population was next infected with a lentivirus driving the expression of the BirA biotin ligase together with the neomycin resistance gene. After amplification of this population, MCF7-rTTA3-BirA cells were infected with lentiviruses prepared with vectors driving the expression of internally Avi-tagged Prescission-cleavable cyclin D3/CDK4 fusion protein or luciferase reporters fused or not to GFP together with the expression of the GFP fluorescent marker (through an IRES sequence).

In order to verify if the inducible Prescission-cleavable biotinylatable cyclin D3/CDK4 fusion proteins with an internal Avi-tag expressed in MCF7 cells cultivated in 96-well microplates after transduction with the vectors described above could be immobilized on streptavidin-coated plates and were enzymatically active, the MCF7 cells expressing the luciferase or the cyclin D3/CDK4 fusion protein described above were cultivated in 96 well microplates with 200 μl DMEM medium supplemented 100 μM Biotin, 5% serum, insulin 6 ng/ml, NEAA (GIBCO) 0.1 mM, Sodium pyruvate (1 mM), Penicillin (50 U/ml) and Streptomycin (50 μg/ml). The expression of the reporter was induced by continuous exposure to 1 μg/ml doxycyclin. At the end of the culture, cells were washed with PBS and lysed on ice in 400 uL lysis buffer (100 mM NaCl, 50 mM Tris-HCl pH 7.5, 0.1% SDS, 1% Na deoxycholate, EDTA 1 mM, EGTA 1 mM, 50 mM NaF, 1 mM Orthovanadate, 1 mM β-glycerophosphate, 1 μg/ml leupeptin, 60 μg/ml Pefabloc and 10% glycerol) as described in Example 10 and stored at −70° C. The total volume of defrozen lysate was then incubated for 3 h at 4° C. under slight shaking on streptavidin-coated 96 well plates (Perkin Elmer) which had been prewashed three times with NP-40 lysis buffer without inhibitors.

The Rb-kinase activity was assayed on the cellular extract bound to the streptavidin-coated plates as described previously (Coulonval et al., supra). After 3 hours of incubation at 4° C., complexes were washed three times with 0.5% NP-40 lysis buffer supplemented with 1 mM DTT and three times with the kinase reaction buffer (50 mM Hepes, pH 7.5, 10 mM KCl, 10 mM MgCl2, 2.5 mM EGTA, 1 mM DTT). Washed complexes were incubated in 40 μl of the kinase reaction buffer containing 2 mM ATP, 0.3 μg of a 48-kDa fragment (aa 773-928) of pRb (Sigma), 10 mM β-glycerophosphate, 0.1 mM orthovanadate, 1 mM NaF, 60 μg/ml pefabloc, and 1 μg/ml leupeptine and incubated for 30 min at 30° C. with continuous gentle agitation. The Rb-kinase assay was stopped after 30 minutes by adding 10 μl of EDTA 120 mM to the reaction mixture. The Rb-kinase assay product was transferred to Nunc immobilizer Gluthatione F96 clear plates (Nunc) together with 50 μl DELFIA Assay buffer (Perkin elmer) supplemented with 0.1% BSA to capture the GST-Rb fragment used as substrate in the Rb-kinase assay. Plates were incubated 1 hour at room temperature under mild shaking before being washed twice with DELFIA wash solution (Perkin Elmer). Plates were then incubated overnight at 4° C. under slight agitation (Vibrax shacker) with 50 μl DELFIA Assay buffer (Perkin Elmer) supplemented with 0.1% BSA and 1/1000 anti P-RB826 antibody (Epitomics). Plates were next washed 4 times with DELFIA wash solution (Perkin Elmer) before being incubated 2 hours under mild shaking at room temperature with 50 μl DELFIA Assay buffer (Perkin Elmer) supplemented with 0.1% BSA and 300 ng/ml Eu-labelled anti rabbit IGg (Perkin Elmer). Plates were next washed 4 times with DELFIA wash solution (Perkin Elmer) before being incubated 5 min under mild shaking at room temperature with DELFIA enhancement solution (Perkin Elmer). Fluorescence of release Eu-cryptates was measured after a delay of 100 μseconds for 400μ seconds on a TECAN Infinite Pro 2000 fluorimeter equipped with a 340 nm excitation filter and a 610 nm emission filter.

The remaining CyclinD3/CDK4 bound to the streptavidin-coated plate was incubated with 50 μl DELFIA Assay buffer (Perkin Elmer) supplemented with 0.1% BSA. Plates were incubated overnight at 4° C. under slight agitation (Vibrax shacker) with 50 μl DELFIA Assay buffer (Perkin Elmer) supplemented with 0.1% BSA and 1/1000 of the DCS-28 monoclonal cyclin D3 antibody (Santa Cruz). Plates were next washed 4 times with DELFIA wash solution (Perkin Elmer) before being incubated 2 hours under mild shaking at room temperature with 50 μl DELFIA Assay buffer (Perkin Elmer) supplemented with 0.1% BSA and 300 ng/ml Eu-labelled anti mouse IGg (Perkin Elmer). Plates were next washed 4 times with DELFIA wash solution (Perkin Elmer) before being incubated 10 min at room temperature with DELFIA enhancement solution (Perkin Elmer). Fluorescence of release Eu-cryptates was measured after a delay of 100μ seconds for 400μ seconds on a TECAN Infinite Pro 2000 fluorimeter equipped with a 340 nm excitation filter and a 610 nm emission filter.

As shown in FIG. 22, Eu cryptate fluorescence is only detected with a Eu-labeled secondary antibody and an anti-P-Rb antibody in the Rb-kinase reaction products immobilized on gluthation-coated plates obtained with extracts of MCF7 cells expressing the wild-type biotinylatable cyclin D3/CDK4 fusion reporters. No phosphorylation of the GST-Rb substrate immobilized on the gluthation plates is detected with extracts of MCF7 cells expressing the biotinylatable luciferase fusion reporters. Presence of Cyclin D3 was also only detected on immobilized extracts of MCF7 cells expressing the wild-type biotinylatable cyclin D3/CDK4 fusion reporters. Interestingly, the cell population used to prepare the extract analyzed above only contained 10% of cells expressing the wild-type biotinylatable cyclin D3/CDK4 fusion reporters as judged by the proportion of cells in the population which are endowed with green fluorescence issued from the expression of EGFP fused to the cyclinD3 and CDK4. Sensitivity of the assay will be strongly enhanced when purified populations of cells will be used.

The Example 26 brings thus the proof of concept that the DELFIA technology is applicable to sensitively detect Rb-kinase activity and cyclin D3/CDK4 fusion presence in extracts immobilized on streptavidin-coated plates of MCF7 cell populations cultivated in 96-well plates generated by lentiviral infection and expressing the BirA biotin ligase and a biotinylatable cyclin D3/CDK4 fusion reporters. Furthermore The Example 26 brings thus the proof of concept that the DELFIA mediated indirect measurement of the activation of the CDK4 part of the cyclin D3/CDK4 fusion by a Rb-kinase assay is sensitive enough to detect the activation of the CDK4 part of the cyclin D3/CDK4 fusion stably expressed in mammalian cells after lentiviral infection in a format amenable to high throughput. Example 26 also suggests that direct detection of specific epitopes of the cyclin D3/CDK4 fusion reporter (such as the T172-phosphorylated CDK4) bound on streptavidin immobilized support is possible. Such immobilized cyclin D3/CDK4 fusion reporter extracted from quiescent and phosphorylated cells could be used to differentiate antibodies able to recognize specifically the T172-phosphorylated CDK4.

### Example 27

### Use of Immobilisable Cyclin D/CDK4 Fusion Proteins as Immunogen to Generate Anti-Phospho-T172-CDK4 Antibodies

The cyclin D/CDK4 fusion protein can be tandemly purified from serum-stimulated eukaryotic cells and used as an immunogen to create anti-phospho CDK4 antibodies.

Indeed, examples 25 and 26 teach that immobilized cyclinD/CDK4 fusions produced in mammalian cells cultivated in bulk or in 96-well plates can be detected using the DELFIA technology or other means known to those skilled in the art by specific antibodies such as the anti-cyclin D3 antibody. Furthermore, example 19 teached us that cyclinD/CDK4 fusions are specifically phosphorylated on the T172 of the CDK4 part of the fusion upon serum stimulation. Combined together these teaching implicate that immobilized cyclin D/CDK4 fusions extracted from quiescent and proliferating mammalian cells and eventually digested with Precision protease can be used to discriminate molecules specifically recognizing the phosphorylated T172 form of CDK4.

A typical assay to identify and characterize anti-phospho CDK4 antibodies would comprise the following steps:
providing eukaryotic cells maintained in a quiescent state, said eukaryotic cells comprising a cyclin D/CDK4 fusion protein, wherein the CDK4 part of the fusion protein is present in a hypophosphorylated form;
providing eukaryotic cells maintained in a serum stimulated state, said eukaryotic cells comprising a cyclin D/CDK4 fusion protein, wherein the CDK4 part of the fusion protein is present in a hyperphosphorylated form;
isolating the cyclin D/CDK4 fusion protein from said eukaryotic cells;
immobilizing the cyclin D/CDK4 fusion protein from said quiescent or serum-stimulated eukaryotic cells on a suitable matrix;
eventually digesting the linker between the cyclin D and the CDK4 parts of the cyclinD/CDK4 fusion with the appropriate protease
contacting the immobilized cyclin D/CDK4 fusion protein from said quiescent or serum-stimulated eukaryotic cells to purified or semi-purified antibody preparation;
contacting a labeled secondary antibody to said purified or semi-purified antibody bound to the immobilized cyclin D/CDK4 fusion protein from said quiescent or serum-stimulated eukaryotic cells;
quantifying the presence of said labeled secondary antibody bound to said purified or semi-purified antibody bound to the immobilized cyclin D/CDK4 fusion protein from said quiescent or serum-stimulated eukaryotic cells;
comparing said quantification of the presence of said labeled secondary antibody bound to said purified or semi-purified antibody bound to the immobilized cyclin D/CDK4 fusion protein from said serum-stimulated eukaryotic cells to said labeled secondary antibody bound to said purified or semi-purified antibody bound to the immobilized cyclin D/CDK4 fusion protein from said quiescent eukaryotic cells;
selecting said purified or semi-purified antibody with quantification of the presence of said labeled secondary antibody bound to said purified or semi-purified antibody bound to the immobilized cyclin D/CDK4 fusion protein from said serum-stimulated eukaryotic cells higher than the quantification of the presence of said labeled secondary antibody to said purified or semi-purified antibody bound to the immobilized cyclin D/CDK4 fusion protein from said quiescent eukaryotic cells;

Alternatively, such an assay would be done in vivo, i.e. using stably transfected Eukaryotic cells, comprising said cyclin D/CDK4 fusion proteins. In such as case, the fusion proteins could be hyperphosphorylated due to serum-activation of the Eukaryotic cells or hypophosphorylated due to Eukaryotic cells being kept quiescent. Isolation of the fusion protein after said stimulation (or non-stimulation) would result in a hyper (or hypo) phosphorylated fusion protein, which can be immmobilised on a matrix and used to screen for and characterize antibodies specifically binding phospho-CDK4.

1. An in vitro assay for determining the activation status of endogenous cyclin-dependent kinase 4 (CDK4) in eukaryotic cells, said assay comprising the steps of:
providing eukaryotic cells maintained in a quiescent state, said eukaryotic cells comprising a cyclin D/CDK4 fusion protein, wherein the CDK4 part of the fusion protein is present in a hypophosphorylated form;
inducing proliferation of said eukaryotic cells;
isolating the cyclin D/CDK4 fusion protein from said eukaryotic cells; and
measuring the activation status of said isolated cyclin D/CDK4 fusion protein, thereby determining the activation status of endogenous CDK4 in said eukaryotic cells.

2. The assay according to claim 1, used to evaluate the effect of a candidate agent on the activation status of endogenous CDK4, wherein prior to or upon inducing proliferation of said eukaryotic cells, the assay comprises the step of incubating said eukaryotic cells with at least one candidate agent.

3. An in vitro assay for determining the activation status of endogenous cyclin-dependent kinase 4 (CDK4) in a eukaryotic cell extract, said assay comprising the steps of:
providing a cyclin D/CDK4 fusion protein, wherein the CDK4 part of the fusion protein is present in a hypophosphorylated form;
contacting the cyclin D/CDK4 fusion protein with a eukaryotic cell extract; and
measuring the activation status of said cyclin D/CDK4 fusion protein, thereby determining the activation status of endogenous CDK4 in said eukaryotic cell extract.

4. The assay according to claim 3, wherein the eukaryotic cell extract is obtained from untreated eukaryotic cells.

5. The assay according to claim 3, wherein the eukaryotic cell extract is obtained from synchronized eukaryotic cells.

6. The assay according to any one of claims 3 to 5, used to evaluate the effect of a candidate agent on the activation status of endogenous CDK4, wherein the eukaryotic cell extract is obtained from eukaryotic cells incubated with at least one candidate agent or wherein the eukaryotic cell extract is directly incubated with at least one candidate agent.

7. The assay according to claim 2 or 6, wherein the at least one candidate agent is selected from the group consisting of a biological sample, a protein, a nucleic acid, an siRNA, a microRNA, a chemical compound, and a small molecule.

8. The assay according to any one of claims 1 to 7, wherein the cyclin D/CDK4 fusion protein is produced in prokaryotic cells.

9. The assay according to any one of claims 1 to 7, wherein the cyclin D/CDK4 fusion protein is produced in eukaryotic cells maintained in a quiescent state.

10. The assay according to any one of claim 1, 2, 6, or 9, wherein the eukaryotic cells are maintained in said quiescent state by culturing the eukaryotic cells in the absence of serum and hormones and optionally in the presence of an anti-estrogen compound, preferably fulvestrant.

11. The assay according to any one of claims 1 to 7, wherein the cyclin D/CDK4 fusion protein is produced in eukaryotic cells and dephosphorylated by a phosphatase, preferably the lambda phosphatase.

12. The assay according to any one of claims 1 to 11, wherein the eukaryotic cells are mammalian cells, preferably the mammalian cell line MCF7.

13. The assay according to any one of claims 1 to 12, wherein the activation status of the isolated cyclin D/CDK4 fusion protein CDK4 is measured by quantifying the Rb kinase activity of the isolated cyclin D/CDK4 fusion protein or by detecting the phosphorylation status of the isolated cyclin D/CDK4 fusion protein, preferably by detecting phosphorylation of the isolated cyclin D/CDK4 fusion protein on T172.

14. The assay according to any one of claims 1, 2, 7 to 13, used to identify an activating kinase of endogenous CDK4 in eukaryotic cells, said assay comprising the steps of:
providing eukaryotic cells maintained in a quiescent state, said eukaryotic cells comprising a cyclin D/CDK4 fusion protein, wherein the CDK4 part of the fusion protein is present in a hypophosphorylated form;
incubating said eukaryotic cells with an siRNA directed against a candidate activating kinase,
inducing proliferation of said eukaryotic cells;
isolating the cyclin D/CDK4 fusion protein from said eukaryotic cells;
measuring the activation status of said isolated cyclin D/CDK4 fusion protein; and
identifying the candidate activating kinase as an activating kinase of endogenous CDK4 when the isolated cyclin D/CDK4 fusion protein is not activated.

15. The assay according to any one of claims 3 to 13, used to identify an activating kinase of endogenous CDK4 in a eukaryotic cell extract, said assay comprising the steps of:
incubating eukaryotic cells with an siRNA directed against a candidate activating kinase,
preparing a eukaryotic cell extract from said eukaryotic cells;
providing a cyclin D/CDK4 fusion protein, wherein the CDK4 part of the fusion protein is present in a hypophosphorylated form;
contacting the cyclin D/CDK4 fusion protein with the eukaryotic cell extract;
measuring the activation status of the cyclin D/CDK4 fusion protein; and
identifying the candidate activating kinase as an activating kinase of endogenous CDK4 when the cyclin D/CDK4 fusion protein is not activated.

16. A reporter molecule for determining the activation status of endogenous CDK4, wherein said reporter molecule comprises a cyclin D/cyclin-dependent-kinase 4 (CDK4) fusion protein and an Avi tag.

17. The reporter molecule according to claim 16, comprising the amino acid sequence of any one of SEQ ID No. 7, SEQ ID NO. 8, SEQ ID NO. 9, SEQ ID NO. 10, SEQ ID NO. 79, or SEQ ID NO. 81.

18. The assay according to any one of claims 1 to 15, or the reporter molecule according to claim 16, wherein cyclin D is selected from cyclin D1, cyclin D2, or cyclin D3.

19. A reporter system comprising the reporter molecule according to claim 16 or 17 and the biotin ligase BirA.

20. Use of a reporter molecule for determining the activation status of endogenous CDK4, wherein said reporter molecule comprises a cyclin D/cyclin-dependent-kinase 4 (CDK4) fusion protein.

21. The use according to claim 20, wherein said reporter molecule further comprises an affinity tag.

22. A kit for determining the activation status of endogenous CDK4 in eukaryotic cells or a eukaryotic cell extract, said kit comprising a eukaryotic cell line and a cyclin D/CDK4 fusion protein.

23. A kit for determining the activation status of endogenous CDK4 in eukaryotic cells or a eukaryotic cell extract, said kit comprising a eukaryotic cell line and a nucleic acid encoding a cyclin D/CDK4 fusion protein.

24. The kit according to claim 23, wherein the genomic material of said eukaryotic cell line comprises said nucleic acid encoding the cyclin D/CDK4 fusion protein.

25. The assay according to any one of claims 1, 2, 7 to 13, used to identify an activating kinase of endogenous CDK4 in eukaryotic cells, said assay comprising the steps of:
providing eukaryotic cells maintained in a quiescent state, said eukaryotic cells comprising a cyclin D/CDK4 fusion protein, wherein the CDK4 part of the fusion protein is present in a hypophosphorylated form;
inducing in said eukaryotic cells the expression of a candidate activating kinase with an expression vector;
inducing proliferation of said eukaryotic cells;
isolating the cyclin D/CDK4 fusion protein from said eukaryotic cells;
measuring the activation status of said isolated cyclin D/CDK4 fusion protein; and
identifying the candidate activating kinase as an activating kinase of endogenous CDK4 when the isolated cyclin D/CDK4 fusion protein is activated.

26. The assay according to any one of claims 3 to 13, used to identify an activating kinase of endogenous CDK4 in a eukaryotic cell extract, said assay comprising the steps of:
inducing in a eukaryotic cells the expression of a candidate activating kinase with an expression vector;
preparing a eukaryotic cell extract from said eukaryotic cells;
providing a cyclin D/CDK4 fusion protein, wherein the CDK4 protein is present in a hypophosphorylated form;
contacting the cyclin D/CDK4 fusion protein with the eukaryotic cell extract;
measuring the activation status of the cyclin D/CDK4 fusion protein; and
identifying the candidate activating kinase as an activating kinase of endogenous CDK4 when the cyclin D/CDK4 fusion protein is activated.

27. The assay according to claim 25 or 26, wherein the expression vector is an inducible expression vector.

28. Use of the assay according to any one of the preceding claims to analyse the influence of a candidate agent, compound, material or composition on CDK4-function ing.

29. The use according to claim 28, wherein said CDK4 functioning is implicated in cell-proliferation, cancer, and other proliferative diseases or disorders, such as restenosis or psoriasis.

30. The use of the cyclin D/CDK4 fusion tandemly purified from serum-stimulated eukaryotic cells as immunogen to create phospho CDK4-specific binding molecules.

31. An assay to identify and characterize phospho CDK4-specific binding molecules comprising the steps of:
providing eukaryotic cells maintained in a quiescent state, said eukaryotic cells comprising a cyclin D/CDK4 fusion protein, wherein the CDK4 part of the fusion protein is present in a hypophosphorylated form;
providing eukaryotic cells maintained in a serum stimulated state, said eukaryotic cells comprising a cyclin D/CDK4 fusion protein, wherein the CDK4 part of the fusion protein is present in a hyperphosphorylated form;
isolating the cyclin D/CDK4 fusion protein from said eukaryotic cells;
immobilizing the cyclin D/CDK4 fusion protein from said quiescent or serum-stimulated eukaryotic cells on a suitable matrix;
contacting the immobilized cyclin D/CDK4 fusion protein from said quiescent or serum-stimulated eukaryotic cells with a candidate binding molecule or binding molecule preparation;
comparing the binding of said a candidate binding molecule or binding molecule preparation to the immobilized cyclin D/CDK4 fusion protein from serum-stimulated eukaryotic cells with the binding of said a candidate binding molecule or binding molecule preparation to the immobilized cyclin D/CDK4 fusion protein from quiescent eukaryotic cells;
selecting the binding molecule or binding molecule preparation that preferably binds to the immobilized cyclin D/CDK4 fusion protein from serum-stimulated eukaryotic cells versus quiescent eukaryotic cells, thereby obtaining a phospho CDK4-specific binding molecule or binding molecule preparation.

32. The use according to claim 30, or the assay according to claim 31, wherein said binding agent is selected from the group comprising: a specific antibody, antigen-binding antibody fragment, nanobody, affybody, an aptamer, a photoaptamer, a spiegelmer, a small molecule, an interacting partner, a specifically binding protein or peptide, a Darpin, an ankyrin, an isotopically labelled tracer or a ligand.

33. The assay according to claim 31, wherein said suitable matrix composed of a molecule coupled to a suitable support, said molecule being adapted to the used affinity purification tag fused to the cyclin D/CDK4 fusion protein

34. The assay according to claim 33, wherein said suitable support is selected from the group comprising: agarose, sepharose, polystyrene, polyethylene, gold (Biacore), 0.8 micron-sized iron, super-paramagnetic, hydrophobic, and polymer-encapsulated (non-exposed iron) beads.

35. The assay according to claim 33, wherein said suitable matrix is selected from the group comprising: avidin, streptavidin, neutravidin, CaptAvidin, Tamavidin, and wherein the Avi tag is used in the cyclinD/CDK4 fusion protein; or
wherein said suitable matrix is glutathione and wherein the GST tag is used in the cyclinD/CDK4 fusion protein; or
wherein said suitable matrix is amylase and wherein the MBP tag is used in the cyclinD/CDK4 fusion protein; or
wherein said suitable matrix is a Ni chelate and wherein the His-tag tag is used in the cyclinD/CDK4 fusion protein; or
wherein said suitable matrix is a chloroalkane linker attached to a variety of useful molecules, such as affinity handles, or solid surfaces and wherein the Halo-tag is used in the cyclinD/CDK4 fusion protein; or
wherein said suitable matrix is an O⁶-benzylguanine or O²-benzylcytosine derivative and wherein the Snap/Clip-tag tag is used in the cyclinD/CDK4 fusion protein.
